(19) United States
(12) Patent Application Publication
(10) Pub. No.: US 2025/0113827 A1
(43) Pub. Date: Apr. 10, 2025

(54) METHYLOBACTERIUM STRAINS FOR MITIGATING METHANE AND METHODS RELATED THERETO

(71) Applicant: NewLeaf Symbiotics, Inc., St. Louis, MO (US)

(72) Inventors: Natalie Breakfield, St. Louis, MO (US); Desmond R. Jimenez, St. Louis, MO (US); David Flack, St. Louis, MO (US)

(21) Appl. No.: 18/689,160

(22) PCT Filed: Sep. 8, 2022
(86) PCT No.: PCT/US2022/076129

§ 371 Date: Mar. 5, 2024

Related U.S. Application Data
(60) Provisional application No. 63/388,461, filed on Jul. 12, 2022.
(60) Provisional application No. 63/289,475, filed on Dec. 14, 2021.
(60) Provisional application No. 63/241,818, filed on Sep. 8, 2021.

(51) Int. Cl. A01N 63/20; A01C 1/06; A01P 21/00; C12N 1/20; C12R 1/01

(52) U.S. Cl. CPC A01N 63/20; A01C 1/06; A01P 21/00; C12N 1/20; C12Y 114/13025; C12R 2001/01

(57) ABSTRACT

Methylobacterium strains that enhance early growth of plants, improve propagation/transplant vigor, increase nutrient uptake, improve stand establishment, improve stress tolerance and/or increase a plant's ability to utilize nutrients are provided herein. Also provided are methods to reduce green-house gas emission and convert methane to methanole with Methylobacterium strains.

### CROSS-REFERENCE TO RELATED APPLICATIONS

This patent application claims the benefit of PCT Application No. PCT/US2022/076129, filed Sep. 8, 2022, which claims benefit of U.S. Provisional Patent Application No. 63/241,818, filed Sep. 8, 2021, U.S. Provisional Patent Application No. 63/289,475, filed Dec. 14, 2021, and U.S. Provisional Patent Application No. 63/388,461, filed Jul. 12, 2022, the entire disclosures of which are incorporated herein by reference.

### SEQUENCE LISTING STATEMENT

A Sequence Listing XML containing the file named “NLSYM7003.WO Sequence Listing.xml” which is 268,780 bytes (measured in MS-Windows®) and created on Sep. 13, 2022, contains 53 nucleic acid sequences and 22 amino acid sequences is provided herewith, and is incorporated herein by reference in its entirety.

### BACKGROUND

Plants require certain macronutrients and micronutrients for growth and metabolism. These elements are generally found in the soil as salts and can be consumed by plants as ions. In agriculture, soil can become depleted of one or more of these nutrients requiring the addition of fertilizers to provide sufficient quantities of the nutrients for crop growth. In hydroponic systems, all nutrients must be supplied to the growing plants and are often the greatest cost for a hydroponic plant production system. Methods of enhancing plant production by improving growth and/or increasing nutrient utilization are desired.

One-carbon organic compounds such as methane and methanol are found extensively in nature, and may be utilized as carbon sources by bacteria. Methanotrophic bacteria include species in the genera Methylobacter, Methylorubrum, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase which incorporates an atom of oxygen from 02 into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Some methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nodulans is a nitrogen-fixing Methylobacterium that is not a PPFM (Sy et al., 2001). Some publications have reported that other Methylobacterium species are capable of fixing nitrogen (Madhaiyan et al. (2015) Biotechnol. Biofuels: 8:222; WO2020245675) although nitrogen fixation pathway genes have not been reported to be present in those species.

All nations have tried to frame a global regime to control green-house gas emissions and to assist with adaptation and yet emissions have continued to increase. Methane is a critical component of Earth's carbon cycle and contributes global warming. Agriculture (e.g., enteric fermentation in livestock, manure management, and rice cultivation) is a contributor to global CH₄ emission. Implementation of a biological methane oxidizing technology has the potential for mitigation of atmospheric methane levels and reduction of green-house gas emissions.

### SUMMARY

Provided herein are Methylobacterium strains and compositions comprising one or more Methylobacterium strains, wherein the strains mitigate methane gas, enhance early growth of plants, improve yield of a crop and/or crop product, improve propagation/transplant vigor, increase nutrient uptake, improve stand establishment, improve stress tolerance, and/or increase a plant's ability to utilize nutrients, such as nitrogen, potassium, sulfur, cobalt, copper, zinc, phosphorus, boron, iron, and manganese, and/or that have ability fixate nitrogen. In certain embodiments, the Methylobacterium mitigates methane by oxidation by a soluble methane monooxygenase (sMMO) encoded by genetic elements in the genome of the Methylobacterium strain. In some embodiments, sMMO encoding genetic elements are present on a plasmid in a methane mitigating Methylobacterium strain. In some embodiments, a Methylobacterium strain capable of mitigating methane comprises genetic elements encoding SEQ ID NOS: 1-4. In some embodiments, a Methylobacterium strain capable of mitigating methane comprises genetic elements encoding SEQ ID NOS: 5-8. In some embodiments, a Methylobacterium strain capable of mitigating methane comprises genetic elements encoding sMMO components having at least 65% identity to SEQ ID NOS: 1-4 or 5-8. In some embodiments, the genetic elements comprise SEQ ID NOS: 9-12. In some embodiments, the genetic elements comprise SEQ ID NOS:13-16. In some embodiments, the genetic elements have at least 65% identity to SEQ ID NOS: 9-12 or 13-16. In some embodiments, a Methylobacterium strain capable of mitigating methane is selected from the group consisting of NLS0770 (NRRL B-68075), NLS0737 (NRRL B-68074), NLS5278 (NRRL-B-68186), NLS5334 (NRRL-B-68187), NLS5480 (NRRL-B-68188), NLS5549 (NRRL-B-68189) and variants thereof. In certain embodiments, a Methylobacterium capable of mitigating methane also provides for at least one plant benefit selected from the group consisting of enhanced early growth of plants, improved yield, improved propagation/transplant vigor, increased nutrient uptake, improved stand establishment, improved stress tolerance, and/or increased ability of the plant to utilize nutrients, such as nitrogen, potassium, sulfur, cobalt, copper, zinc, phosphorus, boron, iron, and manganese. In some embodiments, a Methylobacterium capable of mitigating methane fixates nitrogen.

In some embodiments, compositions comprising a Methylobacterium capable of mitigating methane further comprise one or more Methylobacterium strains that provide for at least one plant benefit selected from the group consisting of enhanced early growth of plants, improved yield of a crop and/or crop product, improved propagation/transplant vigor, increased nutrient uptake, improved stand establishment, improved stress tolerance, improved pest resistance, improved pathogen resistance, fruit ripening and/or increased ability of the plant to utilize nutrients, such as nitrogen, potassium, sulfur, cobalt, copper, zinc, phosphorus, boron, iron, and manganese. In some embodiments, such compositions comprise a Methylobacterium that fixates nitrogen. In some embodiments, an additional Methylobacterium strain in a composition is selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof. In certain embodiments, a variant of an additional Methylobacterium or variant thereof is identified by the presence of one or more of SEQ ID NOs: 33-75. In certain embodiments, the compositions provided herein enhance uptake and/or utilization of one or more nutrients and/or enhances nitrogen use efficiency of a treated plant or a plant grown in treated soil, and a Methylobacterium in the composition is selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof. In certain embodiments, a Methylobacterium or variant thereof that increases uptake and/or utilization of one or more nutrients and/or enhances nitrogen use efficiency is identified by the presence of one or more of SEQ ID NOs: 33-39, 46-60, 64-66, or 70-75. In certain embodiments, the Methylobacterium in the compositions provided herein comprise one or more genetic elements associated with the ability to enhance early plant growth, wherein the one or more genetic elements (i) is recD2_2 or pinR; or (ii) the one or more genetic elements encode a protein having a consensus amino acid sequence of SEQ ID NO: 17 to SEQ ID NO: 23. In some embodiments, Methylobacterium in the compositions provided herein that improve early plant growth also impart one or more additional beneficial traits to treated plants or plants grown from treated plant parts or seeds, wherein the trait is enhanced uptake of nutrients, enhanced assimilation of nutrients, and/or enhanced nutrient use efficiency. In some embodiments, plants treated with Methylobacterium isolates provided herein demonstrate enhanced nitrogen use efficiency. In certain embodiments, the compositions provided herein enhance yield of a treated crop or crop product of a treated crop plant or a crop plant grown in treated soil. In certain embodiments, the crop is rice, and the compositions comprise a Methylobacterium capable of mitigating methane selected from the group consisting of NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof, and an additional Methylobacterium providing for enhanced yield of rice. In some embodiments, the additional Methylobacterium is selected from the group consisting of LGP2016 (NRRL B-67341), LGP2017 (NRRL B-67741), LGP2019 (NRRL B-67743), and variants thereof. Also provided are isolated Methylobacterium selected from NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof, compositions comprising such Methylobacterium isolates or variants thereof, and plants, plant parts, or seeds that are at least partially coated with compositions comprising NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof. Variants of NLS0737 or NLS0770 can be identified, for example, by the presence of SEQ ID NO:31 in the genome of a methane mitigating Methylobacterium. Variants of NLS5278, NLS5334, NLS5480, or NLS5549 can be identified, for example, by the presence of SEQ ID NO:32 in the genome of a methane mitigating Methylobacterium. In some embodiments, the plant is rice. In some embodiments, the plant is a crop grown for feeding livestock, for example grasses in a pasture where livestock feed. In some embodiments, the coated plant or plant part comprises plant material harvested for livestock feed, wherein the Methylobacterium is applied to a seed or to a growing a plant, and wherein harvested plant material comprises methane mitigating Methylobacterium. In some embodiments, Methylobacterium is added directly to livestock feed.

Also provided are compositions comprising a fermentation product comprising a Methylobacterium strain that is essentially free of contaminating microorganisms. In certain embodiments, the Methylobacterium strain is selected from the group consisting of NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof. In certain embodiments, the composition further comprises one or more additional components including one or more agriculturally acceptable adjuvants or excipients, and/or an additional active component, for example a pesticide or a second biological. In certain embodiments, the pesticide can be, for example, an insecticide, a fungicide, an herbicide, or a nematicide. The second biological can be a strain that improves yield or controls an insect, pest, fungi, weed, or nematode. In some embodiments, a second biological is a second Methylobacterium strain.

Also provided herein are plants, plant parts or seeds that are treated with Methylobacterium strains and compositions provided herein. Such plants can be without limitation, agricultural crop plants, fruits and vegetables, leafy green plants, herbs, ornamentals, turf grasses and trees.

Also provided herein are methods of mitigating methane using Methylobacterium strains and compositions provided herein. Methane mitigation methods provided herein include methods to decrease methane levels by reducing methane emissions or by enhancing removal of methane from sources of the gas, such as agricultural soil, wetlands, landfills, waste facilities, animal feed, water or air. In one embodiment, a method for mitigating methane gas in an agricultural field that comprise applying a composition to a field, plant, plant part or seed, wherein the composition comprises at least one Methylobacterium selected from the group consisting of NLS0737, NLS0770, NLS5278, NLS5334, NLS5480, NLS5549, and variants thereof, and growing the Methylobacterium whereby the Methylobacterium uses methane as a carbon source, and said utilization of the methane as the carbon source oxidizes methane and reduces methane emissions in the field. In some embodiments, the Methylobacterium composition is applied to an irrigated field, a flooded field, or a field that will be irrigated or will become flooded. In some embodiments, the Methylobacterium is applied to a rice plant, plant part or seed. In some embodiments, the Methylobacterium is applied to a flooded or irrigated rice field.

In some embodiments, a method for mitigating methane comprises treating a pasture, wasteland, a landfill or waste with a composition comprising at least one Methylobacterium isolate; and growing the Methylobacterium in the pasture, wasteland, a landfill or waste thereby mitigating methane. In some embodiments, the Methylobacterium is selected from the group consisting of NLS0737, NLS0770, NLS5278, NLS5334, NLS5480, NLS5549, and variants thereof.

In some embodiments, a method for mitigating methane comprises mitigation of methane production by livestock, wherein said method comprises treating land where livestock feed or will feed, with at least one Methylobacterium thereby reducing methane from livestock feed. In some embodiments, the Methylobacterium is selected from the group consisting of NLS0737, NLS0770, NLS5278, NLS5334, NLS5480, NLS5549, and variants thereof.

Also, methods for reducing methane emissions from a methane emitting source are disclosed. These methods comprise applying a composition comprising at least one Methylobacterium isolate to the methane emitting source.

In some embodiments, a method for mitigating methane comprises reducing methane concentration in a methane-containing media (e.g., manure or livestock waste) or fluid (e.g., any methane-containing gas or liquid such as methane-contaminated groundwater), the method comprising applying a composition comprising at least one Methylobacterium isolate to the media or fluid. In some embodiments, the Methylobacterium is selected from the group consisting of NLS0737, NLS0770, NLS5278, NLS5334, NLS5480, NLS5549, and variants thereof.

In some embodiments, a method for mitigating methane comprises reducing methane emissions (e.g., in a landfill), the method comprising applying a first coating of a composition comprising at least one Methylobacterium isolate to a first layer of material (e.g., overburden/soil or waste); at least partially covering the first layer and first coating with a second layer of material (e.g., overburden/soil or additional waste); applying a second coating of the composition comprising the at least one Methylobacterium isolate to a second layer; and growing the Methylobacterium. In some embodiments, the Methylobacterium is selected from the group consisting of NLS0737, NLS0770, NLS5278, NLS5334, NLS5480, NLS5549, and variants thereof.

Also disclosed is a method for selecting a Methylobacterium isolate capable of utilizing methanol as a food source, wherein the method comprises (a) detecting in the genome of a Methylobacterium isolate, a genetic element, wherein the genetic element comprises a component of a soluble methane monooxygenase; and (b) treating a field, water, plant, plant part or seed with the Methylobacterium isolate, and measuring green-house gas emissions.

Also provided herein are recombinant constructs for expression of an sMMO component protein, or modification thereof, wherein said construct comprises a genetic element encoding any one or more of SEQ ID NO: 1-8 or a modification thereof.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

As used herein, the term “biological” refers to a component of a composition for treatment of plants or plant parts comprised of or derived from a microorganism. Biologicals include biocontrol agents, other beneficial microorganisms, microbial extracts, plant extracts, yeast extracts, vegetal chitosan, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

As used herein “mitigate methane” refers to decreasing methane levels by reducing methane emissions or by enhancing removal of methane from sources of the gas, such as agricultural soil, wetlands, landfills, waste facilities, animal feed, water or air. Mitigation of methane may be the result of methane oxidation by the activity of sMMO in the Methylobacterium strains provided herein, or may be the result of secondary effects of the provided Methylobacterium strains on the microbiome of a treated plant or plant part.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); Methylobacterium symbiota; or Methylobacterium organophilum.

“Colonization efficiency” as used herein refers to the relative ability of a given microbial strain to colonize a plant host cell or tissue as compared to non-colonizing control samples or other microbial strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

As used herein “mineral nutrients” (also sometime referred to simply as “nutrients”) are micronutrients or macronutrients required or useful for plants or plant parts including for example, but not limited to, nitrogen (N), potassium (K), calcium (Ca), magnesium (Mg), phosphorus (P), and sulfur (S), and the micronutrients chlorine (Cl), Iron (Fe), Boron (B), manganese (Mn), zinc (Z), cobalt (Co), copper (Cu), molybdenum (Mo) and nickel (Ni).

As used herein, “vitamins” are organic compounds required in small amounts for normal growth and metabolism. Vitamins are important for human and/or animal growth and some vitamins have been reported to be beneficial to plants. Vitamins include but are not limited to vitamin A (including but not limited to all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

As used herein “fertilizer” can be a single nutrient nitrogen fertilizer, such as urea, ammonia or ammonia solutions (including ammonium nitrate, ammonium sulfate, calcium ammonium nitrate, and urea ammonium nitrate). In certain embodiments, the fertilizer can be a single nutrient phosphate fertilizer, such as a superphosphate or triple superphosphate or mixtures thereof, including double superphosphate. In certain embodiments, the fertilizer can be a single nutrient potassium-based fertilizer, such as muriate of potash. In certain embodiments, the compositions comprise multinutrient fertilizers including binary fertilizers (NP, NK, PK), including, for example monoammonium phosphate, diammonium phosphate, potassium nitrate and potassium chloride. In further embodiments, three-component fertilizers (NPK) providing nitrogen, phosphorus, and potassium are present in the aqueous compositions. In still further embodiments, the fertilizer comprises micronutrients, which may be chelated or non-chelated. In some embodiments, combinations of various fertilizers can be present in the aqueous solution, including combinations of nitrogen, phosphorus and/or micronutrient fertilizers. Nutrient solutions provided in hydroponic plant growth systems are also considered “fertilizers” in methods and compositions described herein.

As used herein, the term “strain” shall include all isolates of such strain.

As used herein, “variant” when used in the context of a Methylobacterium isolate, refers to any isolate that has chromosomal genomic DNA with at least 99%, 99.9, 99.8, 99.7, 99.6%, or 99.5% sequence identity to chromosomal genomic DNA of a reference Methylobacterium isolate, such as, for example, a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinidis et al. (2005)) using sequence analysis tools, such as BLAST, as taught by Altschul et al. (1990) or clustalw (www.ebi.ac.uk/Tools/msa/clustalw2/).

As used herein, “derivative” when used in the context of a Methylobacterium isolate, refers to any Methylobacterium that is obtained from a deposited Methylobacterium isolate provided herein. Derivatives of a Methylobacterium isolate include, but are not limited to, derivatives obtained by selection, derivatives selected by mutagenesis and selection, and genetically transformed Methylobacterium obtained from a Methylobacterium isolate. A “derivative” can be identified, for example based on genetic identity to the strain or isolate from which it was obtained and will generally exhibit chromosomal genomic DNA with at least 99%, 99.9, 99.8, 99.7, 99.6%, or 99.5% sequence identity to chromosomal genomic DNA of the strain or isolate from which it was derived.

As used herein, “sequence identity” when used to evaluate whether a particular Methylobacterium strain is a variant or derivative of a Methylobacterium strain provided herein refers to a measure of nucleotide-level genomic similarity between the coding regions of two genomes. Sequence identity between the coding regions of bacterial genomes can be calculated, for example, by determining the Average Nucleotide Identity (ANI) score using FastANI (Jain et al. “High throughput ANI analysis of 90K prokaryotic genomes reveals clear species boundaries”, Nat Communications 9, 5114 (2018)) and Han et al. (“ANI tools web: a web tool for fast genome comparison within multiple bacterial strains”; Database, 2016, 1-5).

As used herein, a “correlation” is a statistical measure that indicates the extent to which two or more variables, here plant growth enhancement and identified genetic elements, occur together. A positive correlation indicates that a microbial strain containing a given genetic element is likely to enhance plant growth.

As used herein, a “pan-genome” is the entire set of genes for the microbial population being screened in a plant colonization efficiency screen. Thus, a pan-genome may represent the entire set of genes for a particular species, or the entire set of genes in multiple different species of the same genus or even the entire set of genes for multiple species classified in more than a single genus, where the strains in the population are from closely related genera.

As used herein a “genetic element” refers to an element in a DNA or RNA molecule that comprises a series of adjacent nucleotides at least 20 nucleotides in length and up to 50, 100, 1,000, or 10,000 or more, nucleic acids in length. A genetic element may comprise different groups of adjacent nucleic acids, for example, where the genome of a plant-associated microorganism contains introns and exons. The genetic element may be present on a chromosome or on an extrachromosomal element, such as a plasmid. In eukaryotic plant-associated microorganisms, the genetic element may be present in the nucleus or in the mitochondria. In some embodiments, the genetic element is a functional genetic element (e.g., a gene) that encodes a protein.

As used herein, the terms “homologous” or “homologue” or “ortholog” refer to related genetic elements or proteins encoded by the genetic elements that are determined based on the degree of sequence identity. These terms describe the relationship between a genetic element or encoded protein found in one isolate, species or strain and the corresponding or equivalent genetic element or protein in another isolate, species or strain. As used herein, a particular genetic element in a first isolate, species or strain is considered equivalent to a genetic element present in a second isolate, species or strain when the proteins encoded by the genetic element in the isolates, species or strains have at least 50 percent identity. Percent identity can be determined using a number of software programs available in the art including BLASTP, ClustalW, ALLALIGN, DNASTAR, SIM, SEQALN, NEEDLE, SSEARCH and the like.

As used herein, the term “cultivate” means to grow a plant. A cultivated plant can be one grown and raised on a large agricultural scale or on a smaller scale, including for example a single plant.

As used herein, the term “hydroponic”, “hydroponics” or “hydroponically” refers to a method of cultivating plants in the absence of soil.

As used herein, the term “mitigating”, “mitigate”, or “mitigation” refers to a reduction of something or a combination of things.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Isolated Methylobacterium strains that mitigate methane directly and/or by impacting the microbiome in a plant environment, are provided herein. Also provided are compositions comprising Methylobacterium strains that mitigate methane and one or more additional Methylobacterium strains that enhance early growth of plants, improve propagation/transplant vigor, increase nutrient uptake, improve stand establishment, improve stress tolerance, increase yield, and/or increase a plant's ability to utilize nutrients, and compositions useful for treatment of fields, wasteland, animal feed, wetlands, landfills, waste, plants, seeds, or plant parts with such strains are provided herein.

Methylobacterium strains that mitigate methane provided herein also provide benefits to a treated plant or a plant grown in treated soil, wherein such benefits include, but are not limited to enhanced early growth of plants, improved propagation/transplant vigor, increased nutrient uptake, improved stand establishment, improved stress tolerance, increased yield, and/or increased ability to utilize nutrients. In some embodiments, methane mitigation by Methylobacterium strains provided herein is the result of enzymatic oxidation of methane by the activity of soluble methane monooxygenae enzyme components encoded by genetic elements present in the Methylobacterium strains. In some embodiments, Methylobacterium strains mitigate methane by impacting other microbial populations or the activity of other microbial populations in the plant environment, for example by increasing activity or populations of methanotrophs, or decreasing the activity or populations of methanogens. In some embodiments, early growth enhancement results in increased yield at harvest, for example increased harvested seed yield.

In certain embodiments, the Methylobacterium in the composition is selected from the group consisting of NLS0737, NLS0770, NLS5278, NLS5334, NLS5480, NLS5549, and variants thereof. In certain embodiments, the composition further comprises one or more Methylobacterium selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof.

In certain embodiments, the plants are grown in a field, an irrigated or flooded field, hydroponically or in an aeroponic plant cultivation system. In certain embodiments, the Methylobacterium in the composition is selected from the group consisting of NLS0737, NLS0770, NLS5278, NLS5334, NLS5480, NLS5549, and variants thereof, and used to treat wetlands, landfills, or waste.

Also provided is an isolated Methylobacterium NLS0737, NLS0770, NLS5278, NLS5334, NLS5480, or NLS5549. In certain embodiments the Methylobacterium in the composition has the ability to mitigate methane directly by oxidation of methane by sMMO. In some embodiments, Methylobacterium strains facilitate oxidization of CH₄ into methanol (CH₃OH) followed by the incorporation of that carbon into bacterial biomass, or its oxidation to CO₂ and H₂O.

In certain embodiments, the Methylobacterium in the composition is NLS0737, NLS0770, NLS5278, NLS5334, NLS5480, NLS5549, or variants thereof. In certain embodiments, the Methylobacterium in the composition has the ability to use methane as a carbon source for growth. Such strains find use as described herein for mitigating methane production, for example in agricultural applications, including plant production in flooded fields, for reducing methane produced in animal production, such as cattle or dairy industries, or for reducing natural methane sources such as exist in wetlands or other natural water sources, (including but not limited to lakes, rivers, mangroves, marshes, bogs and streams), in geological sources, or in gases produced as the result of wildfires, wild animals, or insects. Such strains find use as described herein for mitigating methane production, for example in wetland, landfill, or waste applications for reducing methane produced in animal production, such as cattle or dairy industries, or for reducing natural methane sources such as exist in wetlands or other natural water sources, (including but not limited to lakes, rivers, marshes, bogs and streams), in geological sources, or in gases produced as the result of wildfires, wild animals, or insects. By reducing methane resulting from such practices or present in such sources, the concentration of atmospheric greenhouse gases can be reduced and decrease the potential for methane to have detrimental effects, particularly in contributing to global warming. In some embodiments, Methylobacterium strains provided herein not only mitigate atmospheric methane levels associated with agricultural crop production, but also provide additional benefits to a treated plant.

Further provided are methods of improving production of plants including rice, by treatment with one or more Methylobacterium strains provided herein. In certain embodiments, a Methylobacterium in the composition is NLS0737, NLS0770, NLS5278, NLS5334, NLS5480, or NLS5549. In certain embodiments, the composition is applied to rice or a field or paddy where rice is grown, and the composition further comprises one of more of LGP2016, LGP2017, and LGP2019. In some embodiments, treatment with Methylobacterium NLS0737, NLS0770, NLS5278, NLS5334, NLS5480, or NLS5549 improves plant production, for example by enhanced early growth of treated plants or plants grown from treated seeds in comparison to an untreated control plant or in comparison to a control plant grown from an untreated seed. Such enhanced early growth is measured, for example, by an increase in biomass of treated plants, including increased shoot, leaf, root, or whole seedling biomass. Increased early growth can result in various improvements in plant production, including for example increased biomass production or yield of harvested plants, increased and/or more uniform fruit production, faster seed set, earlier maturation, increased rate of leaf growth, increased rate of root growth, increased seed yield, and decreased cycle time in comparison to an untreated control plant or in comparison to a control plant grown from an untreated seed. In certain embodiments, application of Methylobacterium strains as provided herein provides for a 1%, 2%, 3%, 4%, 5%, 6%, 7%, 8%, 9%, 10%, 12%, 15%, 17%, 20%, 30% or 40% increase in any of the aforementioned traits in comparison to an untreated control plant or in comparison to a control plant grown from an untreated seed. In some embodiments, production is enhanced by increased rooting, for example of plant cuttings, where such increased rooting can result in decreased cycling time and/or increased biomass or yield of the treated plants.

In some embodiments, compositions comprising a Methylobacterium capable of mitigating methane further comprise one or more Methylobacterium strains that provide for at least one plant benefit selected from the group consisting of enhanced early growth of plants, improved yield of a crop and/or crop product, improved propagation/transplant vigor, increased nutrient uptake, improved stand establishment, improved stress tolerance, improved pest resistance, improved pathogen resistance, fruit ripening and/or increased ability of the plant to utilize nutrients, such as nitrogen, potassium, sulfur, cobalt, copper, zinc, phosphorus, boron, iron, and manganese. In some embodiments, such compositions comprise a Methylobacterium that fixates nitrogen.

In some embodiments, an additional Methylobacterium strain in a composition is selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof.

In certain embodiments, the compositions provided herein enhance uptake and/or utilization of one or more nutrients and/or enhances nitrogen use efficiency of a treated plant or a plant grown in treated soil, and a Methylobacterium in the composition is selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof.

In certain embodiments, the Methylobacterium in the compositions provided herein comprise one or more genetic elements associated with the ability to enhance early plant growth, wherein the one or more genetic elements (i) is recD2_2 or pinR; or (ii) the one or more genetic elements encode a protein having a consensus amino acid sequence of SEQ ID NO: 17 to SEQ ID NO: 23. In some embodiments, Methylobacterium in the compositions provided herein that improve early plant growth also impart one or more additional beneficial traits to treated plants or plants grown from treated plant parts or seeds, wherein the trait is enhanced uptake of nutrients, enhanced assimilation of nutrients, and/or enhanced nutrient use efficiency. In some embodiments, plants treated with Methylobacterium isolates provided herein demonstrate enhanced nitrogen use efficiency. In certain embodiments, the compositions provided herein enhance yield of a treated crop or crop product of a treated crop plant or a crop plant grown in treated soil. In certain embodiments, the crop is rice, and the compositions comprise a Methylobacterium capable of mitigating methane selected from the group consisting of NLS0770 (NRRL-B-68075), NLS5278 (NRRL-B-68186), NLS5334 (NRRL-B-68187), NLS5480 (NRRL-B-68188), NLS5549 (NRRL-B-68189), and variants thereof, and an additional Methylobacterium providing for enhanced yield of rice. In some embodiments, the additional Methylobacterium is selected from the group consisting of LGP2016 (NRRL B-67341), LGP2017 (NRRL B-67741), LGP2019 (NRRL B-67743), and variants thereof.

Various methods for identifying a Methylobacterium strain that mitigate methane are also provided herein. In one method, a wetland, field, plant, plant part or seed is treated with at least a first Methylobacterium strain and methane emissions measured and compared to emissions from control strains and/or other tested strains to identify strains that mitigate methane. In some embodiments, Methylobacterium isolates selected for testing in such methods comprise one or more genetic elements encoding protein components of soluble methane monooxygenase.

Methane utilizing strains for use in the methods described herein may be identified by the presence of one or more genes encoding a protein component of soluble methane monooxygenase (sMMO). Soluble MMO typically contains multiple protein components: a reductase “C” component, a regulatory “B” component and a hydroxylase “A” component containing alpha and beta chains. In some embodiments, a Methylobacterium strain capable of mitigating methene is identified by the presence of a gene encoding a methane monooxygenase component protein having an amino acid sequence that has 65% to 100% identity to an sMMO component protein amino acid sequence of SEQ ID NOS: 1-8. Strains NLS0737 and NLS0770 have sequences encoding proteins with amino acid sequences SEQ ID NOS: 1-4. Strains NLS5278, NLS5334, NLS5480, and NLS5549 have sequences encoding proteins with SEQ ID NOS: 5-8. In some embodiments, a Methylobacterium strain having the ability to use methane as a carbon source for growth is identified using an assay to demonstrate growth of the Methylobacterium strain on methane as the sole carbon source. In some embodiments, a Methylobacterium strain for use in mitigating methane emissions is NLS0737, NLS0770, NLS5278, NLS5334, NLS5480, or NLS5549, or variants thereof.

Genetic elements and encoded proteins correlated with methane oxidation described herein were identified by screening a population of Methylobacterium strains and identifying strains that contain a methane monooxygenase (hits) and strains which lack a methane monooxygenase (non-hits). Genetic elements encoding methane monooxygenase component proteins are provided herein as SEQ ID NOS: 9-16. Additional Methylobacterium strains capable of mitigating methane can be identified by the presence of one or more encoding sequences of SEQ ID NOs:9-16, or encoding sequences having from 65-100% identify to SEQ ID NOs:9-16. Strains NLS0737 and NLS0770 comprise sequences with high identity to SEQ ID NOs: 5-8. Strains NLS5278, NLS5334, NLS5480, and NLS5549 comprises sequences high identity to SEQ ID NOS: 13-16. Such genetic elements can be used, for example to generate modifications of sMMO component proteins having improved activity, to transform Methylobacterium strains which lack sMMO genes to generate additional methane oxidizing strains, and to provide or improve methane oxidation capability in non-Methylobacterium microbes. Recombinant expression constructs can be generated for expression of one or more of the proteins encoded by SEQ ID NOS: 1-8 or improved modifications thereof in a microbial cell. SEQ ID NOS: 9-16 or variations thereof having from 65-100% identify to SEQ ID NOs:9-16 find use in such constructs.

In some embodiments, sMMO protein component encoding genetic elements are encoded on one or more plasmids present in a Methylobacterium strain capable of oxidizing methane. In such embodiments, sMMO encoding sequences can be transferred using mobilized plasmids to Methylobacterium strains which lack sMMO genes to generate transconjugant methane oxidizing Methylobacterium strains. Synthetic vector systems or native mobilizable plasmids are useful in generation of transformed microbial strains. In one embodiment, a synthetic vector system will include ColE1 for high-copy maintenance and cloning in E. Coli, an antibiotic selection marker, oriV for Methylobacterium compatible origin of replication, oriT for mobilization and conjugative transfer between bacterial hosts, and a module providing for expression of sMMO protein components expressed by native or heterologous expression elements. Heterologous expression elements that find use in such synthetic vector systems include promoters from phage such as the phage PR, T5 and Sp6 promoters, promoters from lac and trp operons and native Methylobacterium promoters, including the promoter for methanol dehydrogenase mxaF1 and others, such as described by Zhang and Lidstrom (2003). Transconjugation and other methods for genetically modifying microbial genomes with sMMO encoding sequences provided herein are described, for example in US20210171961, the content of which is incorporated herein by reference in its entirety.

Also provided herein are methods of mitigating methane, enhancing growth and/or yield of a plant, comprising treating a plant or soil where said a plant is growing or will be grown, with a Methylobacterium isolate that uses methane as a carbon source selected from the group consisting of NLS0770 (NRRL B-68075), NLS0737 (NRRL B-68074), NLS5278, NLS5334, NLS5480, NLS5549, and variants thereof, and/or a Methylobacterium isolate that enhances uptake and/or utilization of one or more nutrient components of a fertilizer that is applied to improve cultivation of said plant. In some embodiments the one or more nutrient components is selected from the group consisting of nitrogen, phosphorus, potassium and iron. In some embodiments, the Methylobacterium isolate is selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof.

In some embodiments, treatment with said Methylobacterium isolates allows for reduced levels of fertilizer or various fertilizer components during cultivation of said plant. In some embodiments, the plant is an agricultural row crop. In some embodiments, a Methylobacterium treated plant can be cultivated using reduced rates of fertilizer as compared to standard application rates for said plant. In some embodiments, fertilizer application can be reduced by 5%, 10%, 15%, 20%, 25%, 30%, 35%, 40% or more. In certain embodiments, application of fertilizer can be reduced by at least 25%. In some embodiments the amount of one or more components of said fertilizer is reduced. In some embodiments levels of nitrogen, phosphorus, potassium and/or iron are reduced by 5%, 10%, 15%, 20%, 25%, 30%, 35%, 40% or more. Optimal fertilizer and/or fertilizer components may vary depending on the crop, soil, geographical location. Optimal fertilizer levels can also be determined experimentally, for example by measuring yield at increasing amounts of fertilizer, where the optimal fertilizer concentration is identified by determining the level after which no further yield advantage is observed. An example of determining the optimal nitrogen level for growth is described in Sharma et al. (Indian J. Genet. (2018) 78:292-301).

In some embodiments, methods for enhancing growth and/or yield of a plant comprise application of a composition comprising one or more Methylobacterium isolates that mitigate methane, selected from the group consisting of NLS0770 (NRRL B-68075), NLS0737 (NRRL B-68074), NLS5278, NLS5334, NLS5480, and NLS5549, and optionally, one or more Methylobacterium isolates selected from the group consisting of LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), NLS0693 (NRRL B-67926), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof and a fertilizer. In some embodiments, the plant is an agricultural row crop. In some embodiments, the plant is a leafy green plant. In some embodiments, a leafy green plant is treated, and the leafy green plant is cultivated in a hydroponic or aeroponic plant growth environment. In some embodiments, the fertilizer, or component of the fertilizer are present at a reduced rate compared to the optimal level for the plant. In some embodiments, the nitrogen level is reduced by 5%, 10%, 15%, 20%, 25%, 30%, 35%, 40% or more.

In some embodiments of methods provided herein, a pasture, wasteland or field is treated. In some embodiments of methods provided herein, treatment is done in a waste facility. In some embodiments of method provided herein, the field is flooded or irrigated. In some embodiments of the method provided herein, a plant seed is treated. In certain other embodiments, a plant seedling or part thereof is treated. In some embodiments, a plant shoot or seedling is treated. In some embodiments, the treated plant is cultivated to the second true leaf stage (V2) and harvested to determine root and shoot biomass and nitrogen levels. In some embodiments, the treated plant is cultivated for 10 to 14 days. In some embodiments, the treated plant is cultivated for 14 to 28 days. In some embodiments, the treated plant is cultivated for 28 or more days prior to harvest and analysis of tissue samples to determine levels of nitrogen and other mineral nutrients. In some embodiments, treated plant seeds or seedlings are cultivated in a hydroponic system or an aeroponic plant growth system. A hydroponics system can be a water culture system, a nutrient film technique, an ebb and flow system, a drip system, or a wick system. In an aeroponic system, plants are grown in an air or mist environment without the use of soil. In some embodiments, the hydroponic or aeroponic system can be a variation of any of these types or a combination of one or more systems. In some embodiments, a hydroponic or aeroponic system is advantageous over a soil based cultivation system for determining effects of Methylobacterium strains due to the presence of fewer background microorganisms. Various inert substrates can be used to support the plants, seedlings and root systems in hydroponic or aeroponic growth, including but not limited to perlite, rockwool, clay pellets, foam cubes, rock, peat moss, or vermiculite.

In some embodiments, a Methylobacterium strain that enhances plant growth or nitrogen use efficiency, is more efficient at colonizing a plant host cell or tissue, as compared to other Methylobacterium strains. Methods for identifying microbial strains having enhanced colonization efficiency are described in WO2020163027 (PCT/US2020/012041), which is incorporated herein by reference in its entirety. In some embodiments, a Methylobacterium strain that mitigates or decreases methane from agriculture lands, also imparts a trait improvement to said plant selected from increased biomass production, decreased cycle time, increased rate of leaf growth, decreased time to develop two true leaves, increased rate of root growth, and increased seed yield.

Various methods of using Methylobacterium strains to mitigate methane, enhance early growth or rooting, improve propagation/transplant vigor, increase nutrient uptake, improve stand establishment, improve stress tolerance and/or increase a plant's ability to uptake and/or utilize nutrients, such as nitrogen, potassium, sulfur, cobalt, copper, zinc, phosphorus, boron, iron and manganese in plants, such as leafy green plants, row crops, cannabis and other specialty crops are provided herein. In certain embodiments, Methylobacterium treatment of a row crop, including but not limited to corn, soybean, rice, canola, and wheat, results in enhanced plant growth and yield. In certain embodiments, the crop is rice and the Methylobacterium is one or more isolates selected from the group consisting of NLS0770, NLS0737, NLS5278, NLS5334, NLS5480, NLS5549, LGP2016 (ISO17), LGP2017 (ISO18), LGP2019 (ISO20) and variants thereof. In certain embodiments, Methylobacterium treatment of soil, agriculture land, including a field or a flooded and irrigated field, a seed, a leaf, a stem, a root, or a shoot can enhance early growth, propagation/transplant vigor, stand establishment, and/or stress tolerance as well as or alternatively enhance nutrient use efficiency.

Alternatively, such Methylobacterium may be applied to soil or other growth medium where plants are grown. Methylobacterium soil treatments or applications can include, but are not limited to, fields (e.g. flooded or irrigated fields), in-furrow applications (e.g., before, during, and/or after seed deposition), soil drenches, distribution of granular or other dried formulations to the soil (e.g., before, during, and/or after seed deposition or plant growth). Methylobacterium treatments for plants grown in hydroponic systems can include seed treatments prior to germination, foliar applications to germinated plants or parts thereof, and applications in a liquid solution used in the hydroponic system. In certain embodiments, Methylobacterium treatment of a plant can include application to the seed, plant, and/or a part of the plant and can thus comprise any Methylobacterium treatment or application resulting in colonization of the plant by the Methylobacterium. In some embodiments, application of Methylobacterium to crops that are propagated by cutting can enhance growth and/or rooting of such plants. Field transplants of such treated and rooted cuttings may demonstrate decreased cycling time, and/or improved biomass and/or yield as a result of such treatments.

Treatments or applications to plants described herein can include, but are not limited to, spraying, coating, partially coating, immersing, and/or imbibing the field, seed, plant or plant parts with the Methylobacterium strains and compositions comprising the same provided herein. In certain embodiments, soil, a seed, a leaf, a stem, a root, a tuber, or a shoot can be sprayed, immersed and/or imbibed with a liquid, semi-liquid, emulsion, or slurry of a composition provided herein.

Also disclosed are a plant, a plant part or a seed at least partially coated with the compositions described herein. In particular, the plant is a rice plant.

In certain embodiments, methane oxidizing Methylobacterium, such as NLS0737, NLS0770, NLS5278, NLS5334, NLS5480, or NLS5549 described herein are applied as foliar sprays to row crops. In some embodiments, the crop is rice, and plants are treated with an initial foliar application at a flooded stage. In some embodiments, foliar applications are made when a rice paddy is at full flood stage. In some embodiments, additional foliar applications of Methylobacterium are made. In some embodiments, a second foliar application of Methylobacterium is made from 20-40 days following the initial application. In some embodiments, Methylobacterium is also applied as a foliar spray prior to the booting stage of development (characterized by swelling of the flag leaf sheath caused by an increase in the size of the panicle). In some embodiments, a foliar spray is applied 14 days prior to booting stage. In some embodiments, Methylobacterium is applied initially as a foliar spray at full flood stage, followed by a second foliar application approximately 4-6 weeks later, for example around 30 days later. In some embodiments, a third foliar application of Methylobacterium is made not later than 14 days prior to booting stage.

Such treatments, applications, seed immersion, or imbibition can be sufficient to provide for mitigation of green-house gas emissions, enhanced early growth and/or increased levels of one or more mineral nutrients and/or vitamins content in harvestable tissue from a treated plant or plant grown from a treated seed in comparison to an untreated plant or plant grown from an untreated seed. Enhanced early growth can lead to further improvements in plant production including an increase in biomass of treated plants, such as increased shoot, root, or whole seedling biomass. Enhanced early growth can result in various additional improvements in plant production, including for example increased yield of harvested plants or harvested plant parts, increased and/or more uniform fruit production, faster seed set, earlier maturation, increased rate of leaf growth, increased rate of root growth, increased seed yield, and decreased cycle time. In certain embodiments, plant seeds or cuttings can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation. Seed treatments can be effected with both continuous and/or batch seed treaters.

In certain embodiments, the coated seeds can be prepared by slurrying seeds with a coating composition comprising a Methylobacterium strain that increases the levels of one or more mineral nutrients and/or vitamins and air-drying the resulting product. Air-drying can be accomplished at any temperature that is not deleterious to the seed or the Methylobacterium, but will typically not be greater than 30 degrees Centigrade. The proportion of coating that comprises the Methylobacterium strain includes, but is not limited to, a range of 0.1 to 25% by weight of the seed or other plant part, 0.5 to 5% by weight of the seed or other plant part, and 0.5 to 2.5% by weight of the seed or other plant part.

In certain embodiments, a solid substance used in the seed coating or treatment will have a Methylobacterium strain that increases mineral nutrient and or vitamin content adhered to a solid substance as a result of being grown in biphasic media comprising the Methylobacterium strain, solid substance, and liquid media. Methods for growing Methylobacterium in biphasic media include those described in U.S. Pat. No. 9,181,541, which is specifically incorporated herein by reference in its entirety. In certain embodiments, compositions suitable for treatment of a seed or plant part can be obtained by the methods provided in U.S. Pat. No. 10,287,544, which is specifically incorporated herein by reference in its entirety. Various seed treatment compositions and methods for seed treatment disclosed in U.S. Pat. Nos. 5,106,648, 5,512,069, and 8,181,388 are incorporated herein by reference in their entireties and can be adapted for treating seeds with compositions comprising a Methylobacterium strain.

In certain embodiments where plant seeds are treated with Methylobacterium compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soybean protein powders, or a combination of protein powders and a lipid, for example lecithin or a vegetable oil. Lubricants can be applied to seeds simultaneously with application of Methylobacterium, or may be mixed with Methylobacterium prior to application of the compositions to the seeds.

In certain embodiments, treated plants are cultivated in a hydroponic system. In some embodiments, plant seeds are treated and plants are grown from the treated seeds continuously in the same cultivation system. In some embodiments, plant seeds are treated and cultivated in a hydroponic nursery to produce seedlings. The seedlings transferred to a different hydroponic system, for example for commercial production of leafy greens. In some embodiments, a Methylobacterium strain that enhances early growth or increases the levels of one or more mineral nutrients and/or vitamins persists in the seedlings transferred to a greenhouse production system and continues to provide advantages such as improved micronutrient and/or vitamin content and/or biomass production, through the further growth of the leafy green plant. In some embodiments, plant seedlings transferred to a greenhouse production system may be further treated with one or more other Methylobacterium strains that increase the levels of one or more mineral nutrients and/or vitamins prior to, during or after transfer to the production system.

In certain embodiments, the composition used to treat the pasture, wasteland, field, seed, plant, or plant part can contain a Methylobacterium strain and an agriculturally acceptable excipient. Agriculturally acceptable excipients include, but are not limited to, woodflours, clays, activated carbon, diatomaceous earth, fine-grain inorganic solids, calcium carbonate and the like. Clays and inorganic solids that can be used with the include, but are not limited to, calcium bentonite, kaolin, china clay, talc, perlite, mica, vermiculite, silicas, quartz powder, montmorillonite and mixtures thereof. Agriculturally acceptable excipients also include various lubricants (which can provide for smooth flow and separation (singulation) of seeds) such as talc, graphite, polyethylene wax based powders (such as Fluency Agent), protein powders, for example soybean protein powders, or a combination of protein powders and a lipid, for example lecithin or a vegetable oil.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use with compositions comprising a suitable Methylobacterium strain. In certain embodiments, the seed and/or seedling is exposed to the composition by providing the Methylobacterium strain in soil in which the plant or a plant arising from the seed are grown, or other plant growth media in which the plant or a plant arising from the seed are grown. Examples of methods where the Methylobacterium strain is provided in the field and soil include in furrow applications, soil drenches, and the like.

In certain embodiments, NLS0737, NLS0770, NLS5278, NLS5334, NLS5480, NLS5549, LGP2022, LGP2023, LGP2021 or variants or combinations thereof will also find use in treatment of other plant species to mitigate methane and/or enhance early growth, including, for example field crops, leafy greens, herbs, ornamentals, turf grasses and trees grown in commercial production, such as conifer trees. Without limitation, such additional plant species include corn, soybean, cruciferous or Brassica sp. vegetables (e.g., B. napus, B. rapa, B. juncea), alfalfa, rice, rye, wheat, barley, oats, sorghum, millet (e.g., pearl millet (Pennisetum glaucum), proso millet (Panicum miliaceum), foxtail millet (Setaria italica), and finger millet (Eleusine coracana)), sunflower, safflower, tobacco, potato, peanuts, cotton, species in the genus Cannabis (including, but not limited to, Cannabis sativa and industrial hemp varieties), alfalfa, clover, cover-crops, sweet potato (Ipomoea batatus), cassava, coffee, coconut, ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass); fruit (including but not limited to citrus, pome, and tropical fruit); nuts; and tea.

Leafy green plants that can be treated include vegetable crop with edible leaves, for example, spinach, kale, lettuce (including but not limited to romaine, butterhead, iceberg and loose leaf lettuces), collard greens, cabbage, beet greens, watercress, swiss chard, arugula, escarole, endive, bok choy and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils.

In certain embodiments, a Methylobacterium strain used to treat a given cultivar or variety of plant seed, plant or plant part can be a Methylobacterium strain that was isolated from a different plant species, or a different cultivar or variety of the plant species being treated, and is thus heterologous or non-resident to the treated plant or plant part.

In certain embodiments, a manufactured combination composition comprising two or more Methylobacterium strains can be used to treat a field, seed or plant part in any of the methods provided herein. Such manufactured combination compositions can be made by methods that include harvesting monocultures of each Methylobacterium strain and mixing the harvested monocultures to obtain the manufactured combination composition of Methylobacterium. In certain embodiments, the manufactured combination composition of Methylobacterium can comprise Methylobacterium isolated from different plant species or from different cultivars or varieties of a given plant.

In certain embodiments, a manufactured combination composition comprising one or more Methylobacterium strains and a second biological can be used to treat a field, seed or plant part in any of the methods provided herein. Such manufactured combination compositions can be made by methods that include harvesting monocultures of each strain and mixing the harvested monocultures to obtain the manufactured combination composition of Methylobacterium. In certain embodiments, the manufactured combination composition of Methylobacterium and the second biological can comprise isolates from different plant species or from different cultivars or varieties of a given plant.

In certain embodiments, an effective amount of the Methylobacterium strain or strains used in treatment of plants, seeds or plant parts is a composition having a Methylobacterium titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an effective amount of the Methylobacterium strain or strains is a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an effective amount of the Methylobacterium strain or strains is a composition with the Methylobacterium at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of the composition. In certain embodiments, an effective amount of a composition provided herein can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of Methylobacterium strain or strains is adhered thereto. In certain embodiments, an effective amount of a composition provided herein to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains adhered to a solid substance is provided therein or grown therein. In certain embodiments, an effective amount of a composition provided herein can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains is provided therein or grown therein.

In certain embodiments, an effective amount of a Methylobacterium strain or strains that provides for mitigation of green-house gas emissions is at least about 10³, 10⁴, 10⁵, or 10⁶ CFU per seed or treated plant part. In certain embodiments, an effective amount of Methylobacterium provided in a treatment of a seed or plant part is at least about 10³, 10⁴, 10⁵, or 10⁶ CFU to about 10⁷, 10⁸, 10⁹, or 10¹⁰ CFU per seed or treated plant part. In certain embodiments, the effective amount of Methylobacterium provided in a treatment of a seed or plant part is an amount where the CFU per seed or treated plant part will exceed the number of CFU of any resident naturally occurring Methylobacterium strain by at least 5-, 10-, 100-, or 1000-fold. In certain embodiments, the effective amount of Methylobacterium provided in a treatment of a seed or plant part is an amount where the CFU per seed or treated plant part will exceed the number of CFU ofany resident naturally occurring Methylobacterium by at least 2-, 3-, 5-, 8-, 10-, 20-, 50-, 100-, or 1000-fold. In certain embodiments where the treated plant is cultivated in a hydroponic system, populations of naturally occurring Methylobacterium or other soil microbes will be minimal.

Non-limiting examples of Methylobacterium strains that can be used in methods provided herein are disclosed in Table 1. Other Methylobacterium strains useful in certain methods provided herein include variants of the Methylobacterium strains disclosed in Table 1. Also of use are various combinations of two or more strains or variants of Methylobacterium strains disclosed in Table 1 for treatment of plants or parts thereof.

TABLE 1
Methylobacterium sp. strain

| | Isolate | LGP | USDA ARS | Strain Source: Obtained |
|---|---|---|---|---|
| Deposit Identifier | No. | NO. | NRRL No.¹ | from: |
| Methylobacterium | ISO01 | LGP2000 | NRRL B-50929 | a soybean plant grown in |
| sp. #1 | | | | Saint Louis County, |
| | | | | Missouri, USA |
| Methylobacterium | ISO02 | LGP2001 | NRRL B-50930 | a weed grown in Saint Louis |
| sp. #2 | | | | County, Missouri, USA |
| Methylobacterium | ISO03 | LGP2002 | NRRL B-50931 | a mint plant grown in Saint |
| sp. #3 | | | | Louis County, Missouri, |
| | | | | USA |
| Methylobacterium | ISO04 | LGP2003 | NRRL B-50932 | a soybean plant grown in |
| sp. #4 | | | | Saint Louis County, |
| | | | | Missouri, USA |
| Methylobacterium | ISO05 | LGP2004 | NRRL B-50933 | a broccoli plant grown in |
| sp. #5 | | | | Saint Louis County, |
| | | | | Missouri, USA |
| Methylobacterium | ISO06 | LGP2005 | NRRL B-50934 | a corn plant grown in Saint |
| sp. #6 | | | | Louis County, Missouri, |
| | | | | USA |
| Methylobacterium | ISO07 | LGP2006 | NRRL B-50935 | a corn plant grown in Saint |
| sp. #7 | | | | Louis County, Missouri, |
| | | | | USA |
| Methylobacterium | ISO08 | LGP2007 | NRRL B-50936 | a corn plant grown in Saint |
| sp. #8 | | | | Louis County, Missouri, |
| | | | | USA |
| Methylobacterium | ISO09 | LGP2008 | NRRL B-50937 | a corn plant grown in Saint |
| sp. #9 | | | | Louis County, Missouri, |
| | | | | USA |
| Methylobacterium | ISO10 | LGP2009 | NRRL B-50938 | a corn plant grown in Saint |
| sp. #10 | | | | Louis County, Missouri, |
| | | | | USA |
| Methylobacterium | ISO11 | LGP2010 | NRRL B-50939 | a lettuce plant grown in Saint |
| sp. #11 | | | | Louis County, Missouri, |
| | | | | USA |
| Methylobacterium | ISO12 | LGP2011 | NRRL B-50940 | a corn plant grown in Saint |
| sp. #12 | | | | Louis County, Missouri, |
| | | | | USA |
| Methylobacterium | ISO13 | LGP2012 | NRRL B-50941 | a tomato plant grown in |
| sp. #13 | | | | Saint Louis County, |
| | | | | Missouri, USA |
| Methylobacterium | ISO14 | LGP2013 | NRRL B-50942 | a tomato plant grown in |
| sp. #14 | | | | Saint Louis County, |
| | | | | Missouri, USA |
| Methylobacterium | ISO15 | LGP2014 | NRRL B-67339 | a soybean plant grown in |
| sp. #15 | | | | Saint Louis County, |
| | | | | Missouri, USA |
| Methylobacterium | ISO16 | LGP2015 | NRRL B-67340 | a yucca plant grown in Saint |
| sp. #16 | | | | Louis County, Missouri, |
| | | | | USA |
| Methylobacterium | ISO17 | LGP2016 | NRRL B-67341 | a soybean plant grown in |
| sp. #17 | | | | Saint Louis County, |
| | | | | Missouri, USA |
| Methylobacterium | ISO18 | LGP2017 | NRRL B-67741 | a Dionaea muscipula plant |
| sp. #18 | | | | (Venus fly trap) grown in St. |
| | | | | Charles, MO. |
| Methylobacterium | ISO19 | LGP2018 | NRRL B-67742 | an Orchidaceae spp. plant |
| sp. #19 | | | | (orchid) grown in Saint |
| | | | | Louis County, Missouri, |
| | | | | USA |
| Methylobacterium | ISO20 | LGP2019 | NRRL B-67743 | a tomato plant grown in |
| sp. #20 | | | | Saint Louis County, |
| | | | | Missouri, USA Saint Louis |
| | | | | County, Missouri, USA |
| Methylobacterium | ISO22 | NLS0497 | NRRL B-67925 | A cup plant (Silphium |
| sp. #22 | | | | perfoliatum in5 |
| Methylobacterium | ISO23 | NLS0693 | NRRL B-67926 | a vinca vine (Vinca minor) in |
| sp. #23 | | | | Saint Louis County, |
| | | | | Missouri, USA |
| Methylobacterium | ISO24 | NLS1179 | NRRL B-67929 | Rainwater collected in Saint |
| sp. #24 | | | | Louis County, Missouri, |
| | | | | USA |
| Methylobacterium | ISO25 | LGP2167 | NRRL B-67927 | An Acer ginnala (Amur |
| sp. #25 | | | | maple) grown in Saint Louis |
| | | | | County, Missouri, USA |
| Methylobacterium | ISO26 | LGP2020 | NRRL-B-67892 | A Lagerstroemia indica |
| sp. #26 | | | | (crape myrtle) plant grown in |
| | | | | Saint Louis County, |
| | | | | Missouri, USA |
| Methylobacterium | ISO28 | LGP2021 | NRRL-B-68032 | A Cichorium intybus |
| sp. #28 | | | | (chicory) plant growing in |
| | | | | Saint Louis County, |
| | | | | Missouri, USA |
| Methylobacterium | ISO29 | LGP2022 | NRRL-B-68033 | A Coronilla vario (crown |
| sp. #29 | | | | vetch) plant growing in Saint |
| | | | | Louis County, Missouri, |
| | | | | USA |
| Methylobacterium | ISO30 | LGP2023 | NRRL-B-68034 | A Catharanthus roseus |
| sp. #30 | | | | (periwinkle) growing in Fort |
| | | | | Myers, Florida, USA |
| Methylobacterium | ISO31 | LGP2028 | NRRL-B-68064 | A Nasturtium spp. growing |
| sp. #31 | | | | in Saint Louis County, |
| | | | | Missouri, USA |
| Methylobacterium | ISO32 | LGP2029 | NRRL B-68065 | A Salvia officinalis (sage) |
| sp #32 | | | | growing in Saint Louis |
| | | | | County, Missouri, USA |
| Methylobacterium | IS033 | LGP2030 | NRRL B-68066 | A Prunus persica (peach, |
| sp #33 | | | | ‘Hale Haven’), growing in |
| | | | | Dudley, Missouri, USA |
| Methylobacterium | ISO34 | LGP2031 | NRRL B-68067 | An Acer spp. (maple) |
| sp #34 | | | | growing in Dudley, |
| | | | | Missouri, USA |
| Methylobacterium | ISO35 | LGP2033 | NRRL B-68068 | A Rosa rugosa (Japanese |
| sp #35 | | | | rose) growing in Camden, |
| | | | | Maine, USA |
| Methylobacterium | IS036 | LGP2034 | NRRL B-68069 | A Solidago sp. (goldenrod) |
| sp #36 | | | | growing in Camden, Maine, |
| | | | | USA |
| Methylobacterium | ISO37 | NLS0737 | NRRL-B-68074 | A lettuce plant growing in |
| sp. #37 | | | | San Luis Obispo, CA |
| Methylobacterium | ISO38 | NLS0770 | NRRL-B-68075 | A lettuce plant growing in |
| sp. #38 | | | | San Luis Obispo, CA |
| Methylobacterium | ISO39 | NLS5278 | NRRL-B-68186 | An unidentified non-crop |
| sp. #39 | | | | plant growing in Durham, |
| | | | | North Carolina, USA |
| Methylobacterium | ISO40 | NLS5334 | NRRL-B-68187 | An unidentified non-crop |
| sp. #40 | | | | plant growing in Durham, |
| | | | | North Carolina, USA |
| Methylobacterium | ISO41 | NLS5480 | NRRL-B-68188 | An unidentified non-crop |
| sp. #41 | | | | plant growing in Waxhaw, |
| | | | | North Carolina, USA |
| Methylobacterium | ISO42 | NLS5549 | NRRL-B-68189 | An unidentified non-crop |
| sp. #42 | | | | plant growing in Waxhaw, |
| | | | | North Carolina, USA |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Variants of a Methylobacterium isolate listed in Table 1 include isolates obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. Variants of NLS0737 or NLS0770 can be identified, for example, by the presence of SEQ ID NO: 31 in the genome of a methane mitigating Methylobacterium. Variants of NLS5278, NLS5334, NLS5480, or NLS5549 can be identified, for example, by the presence of SEQ ID NO: 32 in the genome of a methane mitigating Methylobacterium.

In certain embodiments of the methods provided herein, the Methylobacterium strain or strains used to treat a plant seed and/or a plant part are selected from the group consisting of ISO01 (NRRL B-50929), IS002 (NRRL B-50930), IS003 (NRRL B-50931), IS004 (NRRL B-50932), ISO05 (NRRL B-50933), IS006 (NRRL B-50934), IS007 (NRRL B-50935), IS008 (NRRL B-50936), IS009 (NRRL B-50937), ISO10 (NRRL B-50938), ISO11 (NRRL B-50939), IS012 (NRRL B-50940), IS013 (NRRL B-50941), IS014 (NRRL B-50942), ISO15 (NRRL B-67339), IS016 (NRRL B-67340), IS017 (NRRL B-67341), IS018 (NRRL B-67741), IS019 (NRRL B-67742), IS020 (NRRL B-67743), IS021 (NRRL B-67892), NLS0770, NLS0737, NLS5278, NLS5334, NLS5480, NLS5549, variants thereof, or any combination thereof. In certain embodiments, one or more of the Methylobacterium strains used in the methods can comprise total genomic DNA (chromosomal and plasmid DNA) or average nucleotide identity (ANI) with at least 99%, 99.9, 99.8, 99.7, 99.6%, or 99.5% sequence identity or ANI to total genomic DNA of ISO01 (NRRL B-50929), IS002 (NRRL B-50930), IS003 (NRRL B-50931), IS004 (NRRL B-50932), ISO05 (NRRL B-50933), IS006 (NRRL B-50934), IS007 (NRRL B-50935), IS008 (NRRL B-50936), IS009 (NRRL B-50937), ISO10 (NRRL B-50938), ISO11 (NRRL B-50939), IS012 (NRRL B-50940), IS013 (NRRL B-50941), IS014 (NRRL B-50942), ISO15 (NRRL B-67339), IS016 (NRRL B-67340), IS017 (NRRL B-67341), IS018 (NRRL B-67741), IS019 (NRRL B-67742), IS020 (NRRL B-67743), IS021 (NRRL B-67892), NLS0770, NLS0737, NLS5278, NLS5334, NLS5480, NLS5549. In certain embodiments, the percent ANI can be determined as disclosed by Konstantinidis et al., 2006. In certain embodiments of the methods provided herein, the Methylobacterium strain or strains used to treat a seed and/or a plant part is NLS0737, NLS0770, NLS5278, NLS5334, NLS5480, or NLS5549 which were deposited under the NRRL accession Nos. NRRL-B-68074, NRRL-B-68075, NRRL-B-68186, NRRL-B-68187, NRRL-B-68188, and NRRL-B-68189 respectively.

In certain embodiments of the methods provided herein, plants, plant seeds and/or plant parts are treated with both a Methylobacterium strain and at least one additional component. In some embodiments an additional component can be an additional active ingredient, for example, a pesticide or a second biological. In certain embodiments, the pesticide can be an insecticide, a fungicide, an herbicide, a nematicide or other biocide. The second biological could be a strain that improves yield or controls an insect, pest, fungi, weed, or nematode. In some embodiments, a second biological is a second Methylobacterium strain.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides, include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a Methylobacterium strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a second biological. The second biological could be a biological control agent, other beneficial microorganisms, microbial extracts, plant extracts, yeast extracts, vegetal chitosan, natural products, plant growth activators or plant defense agent. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1. In certain embodiments, the second biological can be a Methylobacterium selected from M. gregans, M. radiotolerans, M. extorquens, M. populi, M. salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In certain embodiments, compositions comprise multiple additional biological ingredients, including consortia comprising combinations of any of the above bacterial or fungal genera or species.

In further embodiments the second biological can be a biostimulant, including but not limited to seaweed extract or hummates, plant growth activators or plant defense agents including, but not limited to harpin, Reynoutria sachalinensis, jasmonate, lipochitooligosaccharides, and isoflavones.

In further embodiments, the second biological can include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

Fields, plants or harvested plant parts having mitigated methane in comparison to a control field, plant, or plant part are provided, as are methods for obtaining and using such plants and plant parts. In certain embodiments, the content of at least mitigated methane is decreased by at least about 0.1%, 5%, 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

Additionally, disclosed are recombinant constructs for expression of an sMMO component protein, or modification thereof, wherein said construct comprises a genetic element encoding any one or more of SEQ ID NO: 1-8 or a modification thereof.

Further, the recombinant constructs described herein can have the genetic element further comprise a genetic element having a sequence any one of SEQ ID NOS: 9-16, or a modification thereof.

The recombinant constructs disclosed above, can also have the genetic element encodes SEQ ID No. 1-4 or SEQ ID No. 5-8.

### DEPOSIT INFORMATION

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339, NRRL B-67340, and NRRL B-67341 were deposited with NRRL on Nov. 18, 2016. Methylobacterium sp. NRRL B-67741, NRRL B-67742, and NRRL B-67743 were deposited with NRRL on Dec. 20, 2018. Methylobacterium sp. NRRL B-67892 was deposited with NRRL on Nov. 26, 2019. Methylobacterium sp. NRRL B-67925, NRRL B-67926 and NRRL B-67927 were deposited with NRRL on Feb. 21, 2020. Methylobacterium sp. NRRL B-67929 was deposited with NRRL on Mar. 3, 2020. Methylobacterium sp. NRRL B-68032, NRRL B-68033 and NRRL B-68034 were deposited with NRRL on May 20, 2021. Methylobacterium sp. NRRL B-68064, NRRL B-68065, NRRL B-68066, NRRL B-68067, NRRL B-68068, and NRRL B-68069 were deposited with NRRL on Sep. 9, 2021. NRRL B-68074 and NRRL B-68075 were deposited with NRRL on Oct. 6, 2021. NRRL B-68186, NRRL B-68187, NRRL B-68188 and NRRL B-68189 were deposited with NRRL on Aug. 3, 2022.

Subject to 37 CFR § 1.808(b), all restrictions imposed by the depositor on the availability to the public of the deposited material will be irrevocably removed upon the granting of any patent from this patent application.

### EXAMPLES

The following examples are given for purely illustrative and non-limiting purposes of the present invention.

### Example 1 Methylobacterium Inoculation Effect on Promotion of Early Rice Growth

Methylobacterium isolates were tested for their ability to enhance early growth of rice seedlings. A randomized complete block design was used, with 12 treatments in each run; 10 unique Methylobacterium isolates, a Methylobacterium positive control, LGP2018, that demonstrated consistent root growth promotion of rice seedlings during assay development and increased yield levels in corn field trials (WO2020117690). The untreated control sample (UTC) was Methylobacterium growth medium applied in the same amount as used for the Methylobacterium isolates. Each treatment level had an n of 10. All 10 blocks were grown in the same growth chamber, and on the same shelf.

### Procedure:

### Media:

0.5× Murashige and Skoog MS agar plates with 0.5% sucrose

### Pre-Planting:

Rice seeds were de-husked. Average 100 seed count is 2018 mg with approximately 21 g of husked rice per run.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least one hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants (approximately V2 stage) were measured as follows.
Plants that were not impeded from growing normally (by physical surroundings unrelated to presence of Methylobacterium) were removed from plates, and the number of seedlings for that plate recorded.
Seedlings were scanned using WinRhizo and the images analyzed to determine root length for each plant.

The results of this experiment are shown below in Table 2.

TABLE 2

| | | | Absolute | Normalized |
|---|---|---|---|---|
| Experiment | | | Root | Root |
| Number | Treatment ID | Treatment | Length (cm) | Length |
| 264PB | 264PB LGP2018 | LGP2018 | 18.82978 | 100 |
| 264PB | 264PB Strain 1 | LGP2025 | 17.39133 | 73.325898 |
| 264PB | 264PB Strain 2 | LGP2073 | 17.19 | 69.59247 |
| 264PB | 264PB Strain 3 | LGP2047 | 16.37316 | 54.44538 |
| 264PB | 264PB Strain 4 | LGP2045 | 15.96066 | 46.796074 |
| 264PB | 264PB Strain 5 | LGP2151 | 15.39851 | 36.371618 |
| 264PB | 264PB Strain 6 | LGP2103 | 15.04489 | 29.814374 |
| 264PB | 264PB Strain 7 | LGP2125 | 14.84019 | 26.018352 |
| 264PB | 264PB Strain 8 | LGP2017 | 14.54892 | 20.61718 |
| 264PB | 264PB Strain 9 | LGP2120 | 13.84252 | 7.517937 |
| 264PB | 264PB Strain 10 | LGP2124 | 13.18279 | −4.715877 |
| 265PB | 265PB Strain 1 | LGP2071 | 14.117796 | 100.010863 |
| 265PB | 265PB LGP2018 | LGP2018 | 14.117132 | 100 |
| 265PB | 265PB Strain 2 | LGP2061 | 12.535499 | 74.124179 |
| 265PB | 265PB Strain 3 | LGP2107 | 11.83976 | 62.741755 |
| 265PB | 265PB Strain 4 | LGP2065 | 9.992807 | 32.52525 |
| 265PB | 265PB Strain 5 | LGP2051 | 9.743358 | 28.444232 |
| 265PB | 265PB Strain 6 | LGP2054 | 8.960485 | 15.636268 |
| 265PB | 265PB Strain 7 | LGP2092 | 8.856461 | 13.934427 |
| 265PB | 265PB Strain 8 | LGP2079 | 8.610079 | 9.903568 |
| 265PB | 265PB Strain 9 | LGP2052 | 7.916505 | −1.443435 |
| 266PB | 266PB Strain 1 | LGP2059 | 15.569966 | 123.451522 |
| 266PB | 266PB Strain 2 | LGP2016 | 14.587924 | 108.443799 |
| 266PB | 266PB LGP2018 | LGP2018 | 14.035398 | 100 |
| 266PB | 266PB Strain 3 | LGP2158 | 13.207394 | 87.346316 |
| 266PB | 266PB Strain 4 | LGP2066 | 12.900975 | 82.663567 |
| 266PB | 266PB Strain 5 | LGP2141 | 11.897894 | 67.334339 |
| 266PB | 266PB Strain 6 | LGP2078 | 10.298694 | 42.8951 |
| 266PB | 266PB Strain 7 | LGP2050 | 10.041706 | 38.967777 |
| 266PB | 266PB Strain 8 | LGP2080 | 9.462625 | 30.118161 |
| 266PB | 266PB Strain 9 | LGP2048 | 9.284123 | 27.390276 |
| 266PB | 266PB Strain 10 | LGP2053 | 7.207347 | −4.347354 |
| 267PB | 267PB Strain 1 | LGP2046 | 14.419073 | 137.78678 |
| 267PB | 267PB LGP2018 | LGP2018 | 12.303465 | 100 |
| 267PB | 267PB Strain 2 | LGP2024 | 11.846345 | 91.835407 |
| 267PB | 267PB Strain 3 | LGP2148 | 10.620679 | 69.94383 |
| 267PB | 267PB Strain 4 | LGP2144 | 9.415631 | 48.420528 |
| 267PB | 267PB Strain 5 | LGP2150 | 9.382432 | 47.827557 |
| 267PB | 267PB Strain 6 | LGP2110 | 9.298016 | 46.319801 |
| 267PB | 267PB Strain 7 | LGP2176 | 8.103827 | 24.990443 |
| 267PB | 267PB Strain 8 | LGP2153 | 7.128328 | 7.567103 |
| 267PB | 267PB Strain 9 | LGP2082 | 6.373293 | −5.91855 |
| 268PB | 268PB Strain 1 | LGP2021 | 15.569966 | 123.451522 |
| 268PB | 268PB Strain 2 | LGP2040 | 14.587924 | 108.443799 |
| 268PB | 268PB LGP2018 | LGP2018 | 14.035398 | 100 |
| 268PB | 268PB Strain 3 | LGP2138 | 13.207394 | 87.346316 |
| 268PB | 268PB Strain 4 | LGP2095 | 12.900975 | 82.663567 |
| 268PB | 268PB Strain 5 | LGP2130 | 11.897894 | 67.334339 |
| 268PB | 268PB Strain 6 | LGP2099 | 10.298694 | 42.8951 |
| 268PB | 268PB Strain 7 | LGP2077 | 10.041706 | 38.967777 |
| 268PB | 268PB Strain 8 | LGP2102 | 9.462625 | 30.118161 |
| 268PB | 268PB Strain 9 | LGP2072 | 9.284123 | 27.390276 |
| 268PB | 268PB Strain 10 | LGP2081 | 7.207347 | −4.347354 |
| 269PB | 269PB LGP2018 | LGP2018 | 16.079324 | 100 |
| 269PB | 269PB Strain 1 | LGP2094 | 15.70514 | 95.501874 |
| 269PB | 269PB Strain 2 | LGP2101 | 15.386634 | 91.673054 |
| 269PB | 269PB Strain 3 | LGP2090 | 14.624067 | 82.506105 |
| 269PB | 269PB Strain 4 | LGP2093 | 12.998755 | 62.967937 |
| 269PB | 269PB Strain 5 | LGP2084 | 12.830224 | 60.942001 |
| 269PB | 269PB Strain 6 | LGP2114 | 12.516872 | 57.175138 |
| 269PB | 269PB Strain 7 | LGP2100 | 11.343389 | 43.068489 |
| 269PB | 269PB Strain 8 | LGP2085 | 9.828333 | 24.855728 |
| 269PB | 269PB Strain 9 | LGP2075 | 7.587342 | −2.08362 |
| 269PB | 269PB Strain 10 | LGP2083 | 7.50976 | −3.016248 |
| 270PB | 270PB Strain 1 | LGP2029 | 14.570904 | 104.017951 |
| 270PB | 270PB LGP2018 | LGP2018 | 14.31934 | 100 |
| 270PB | 270PB Strain 2 | LGP2135 | 13.363759 | 84.737607 |
| 270PB | 270PB Strain 3 | LGP2129 | 12.594344 | 72.448632 |
| 270PB | 270PB Strain 4 | LGP2143 | 10.608781 | 40.735534 |
| 270PB | 270PB Strain 5 | LGP2137 | 10.04973 | 31.806444 |
| 270PB | 270PB Strain 6 | LGP2128 | 9.970479 | 30.540667 |
| 270PB | 270PB Strain 7 | LGP2123 | 9.933589 | 29.951459 |
| 270PB | 270PB Strain 8 | LGP2126 | 9.635704 | 25.193695 |
| 270PB | 270PB Strain 9 | LGP2136 | 9.506136 | 23.124249 |
| 270PB | 270PB Strain 10 | LGP2121 | 7.872883 | −2.961817 |
| 271PB | 271PB LGP2018 | LGP2018 | 18.545695 | 100 |
| 271PB | 271PB Strain 1 | LGP2069 | 16.856945 | 83.10707 |
| 271PB | 271PB Strain 2 | LGP2027 | 15.948911 | 74.02381 |
| 271PB | 271PB Strain 3 | LGP2056 | 14.750148 | 62.03233 |
| 271PB | 271PB Strain 4 | LGP2096 | 14.330543 | 57.83493 |
| 271PB | 271PB Strain 5 | LGP2060 | 13.874818 | 53.27622 |
| 271PB | 271PB Strain 6 | LGP2097 | 13.443795 | 48.9646 |
| 271PB | 271PB Strain 7 | LGP2067 | 13.24211 | 46.9471 |
| 271PB | 271PB Strain 8 | LGP2055 | 12.770669 | 42.23118 |
| 271PB | 271PB Strain 9 | LGP2086 | 12.549608 | 40.01986 |
| 271PB | 271PB Strain 10 | LGP2057 | 11.572393 | 30.24456 |
| 273PB | 273PB LGP2018 | LGP2018 | 13.216513 | 100 |
| 273PB | 273PB Strain 1 | LGP2028 | 11.289892 | 71.38989 |
| 273PB | 273PB Strain 2 | LGP2098 | 10.957287 | 66.45074 |
| 273PB | 273PB Strain 3 | LGP2116 | 10.552009 | 60.43241 |
| 273PB | 273PB Strain 4 | LGP2131 | 10.492209 | 59.54438 |
| 273PB | 273PB Strain 5 | LGP2117 | 9.92343 | 51.09808 |
| 273PB | 273PB Strain 6 | LGP2133 | 9.207299 | 40.46361 |
| 273PB | 273PB Strain 7 | LGP2140 | 9.188468 | 40.18397 |
| 273PB | 273PB Strain 8 | LGP2134 | 8.651127 | 32.20451 |
| 273PB | 273PB Strain 9 | LGP2109 | 7.244746 | 11.31992 |
| 273PB | 273PB Strain 10 | LGP2111 | 5.404409 | −16.0089 |
| 274PB | 274PB Strain 1 | LGP2033 | 17.459903 | 136.108331 |
| 274PB | 274PB Strain 2 | LGP2118 | 15.623786 | 106.167536 |
| 274PB | 274PB LGP2018 | LGP2018 | 15.245562 | 100 |
| 274PB | 274PB Strain 3 | LGP2145 | 14.631981 | 89.994584 |
| 274PB | 274PB Strain 4 | LGP2032 | 14.299443 | 84.572029 |
| 274PB | 274PB Strain 5 | LGP2152 | 13.881329 | 77.754029 |
| 274PB | 274PB Strain 6 | LGP2147 | 13.409769 | 70.064484 |
| 274PB | 274PB Strain 7 | LGP2157 | 11.306689 | 35.770445 |
| 274PB | 274PB Strain 8 | LGP2142 | 10.1196 | 16.413079 |
| 274PB | 274PB Strain 9 | LGP2159 | 9.361136 | 4.045128 |
| 274PB | 274PB Strain 10 | LGP2154 | 8.943802 | −2.760155 |
| 275PB | 275PB LGP2018 | LGP2018 | 18.826053 | 100 |
| 275PB | 275PB Strain 1 | LGP 2022 | 17.00802 | 80.576456 |
| 275PB | 275PB Strain 2 | LGP2023 | 16.310993 | 73.129541 |
| 275PB | 275PB Strain 3 | LGP2160 | 15.87016 | 68.41976 |
| 275PB | 275PB Strain 4 | LGP2163 | 15.337422 | 62.728087 |
| 275PB | 275PB Strain 5 | LGP2167 | 15.162438 | 60.858589 |
| 275PB | 275PB Strain 6 | LGP2166 | 14.298438 | 51.627764 |
| 275PB | 275PB Strain 7 | LGP2161 | 13.02194 | 37.989883 |
| 275PB | 275PB Strain 8 | LGP2162 | 11.85523 | 25.52496 |
| 275PB | 275PB Strain 9 | LGP2168 | 10.190812 | 7.742619 |
| 277PB | 277PB LGP2018 | LGP2018 | 15.854562 | 100 |
| 277PB | 277PB Strain 1 | LGP2062 | 14.420103 | 81.45296 |
| 277PB | 277PB Strain 2 | LGP2185 | 14.124727 | 77.63385 |
| 277PB | 277PB Strain 3 | LGP2063 | 13.598758 | 70.83327 |
| 277PB | 277PB Strain 4 | LGP2074 | 12.56993 | 57.53088 |
| 277PB | 277PB Strain 5 | LGP2058 | 12.237293 | 53.23002 |
| 277PB | 277PB Strain 6 | LGP2064 | 11.790611 | 47.45458 |
| 277PB | 277PB Strain 7 | LGP2091 | 11.598483 | 44.97043 |
| 277PB | 277PB Strain 8 | LGP2186 | 10.193847 | 26.809 |
| 277PB | 277PB Strain 9 | LGP2105 | 10.166668 | 26.45758 |
| 277PB | 277PB Strain 10 | LGP2187 | 10.018778 | 24.54541 |
| 282PB | 282PB LGP2018 | LGP2018 | 17.115992 | 100 |
| 282PB | 282PB Strain 1 | LGP2087 | 15.150588 | 77.27183 |
| 282PB | 282PB Strain 2 | LGP2108 | 14.929319 | 74.71305 |
| 282PB | 282PB Strain 3 | LGP2076 | 14.913514 | 74.53028 |
| 282PB | 282PB Strain 4 | LGP2106 | 13.131888 | 53.92734 |
| 282PB | 282PB Strain 5 | LGP2113 | 12.547632 | 47.17093 |
| 282PB | 282PB Strain 6 | LGP2049 | 12.529399 | 46.96009 |
| 282PB | 282PB Strain 7 | LGP2068 | 12.507406 | 46.70576 |
| 282PB | 282PB Strain 8 | LGP2149 | 12.28271 | 44.10735 |
| 282PB | 282PB Strain 9 | LGP2005 | 11.888991 | 39.55433 |
| 282PB | 282PB Strain 10 | LGP2006 | 10.285192 | 21.00781 |
| 283PB | 283PB Strain 1 | LGP2182 | 14.59702 | 103.904114 |
| 283PB | 283PB LGP2018 | LGP2018 | 14.364828 | 100 |
| 283PB | 283PB Strain 2 | LGP2034 | 13.842152 | 91.211673 |
| 283PB | 283PB Strain 3 | LGP2146 | 12.351052 | 66.14017 |
| 283PB | 283PB Strain 4 | LGP2181 | 12.117376 | 62.211111 |
| 283PB | 283PB Strain 5 | LGP2089 | 11.13865 | 45.754717 |
| 283PB | 283PB Strain 6 | LGP2156 | 10.858914 | 41.051207 |
| 283PB | 283PB Strain 7 | LGP2170 | 10.110786 | 28.472101 |
| 283PB | 283PB Strain 8 | LGP2155 | 9.582397 | 19.587708 |
| 283PB | 283PB Strain 9 | LGP2127 | 8.857205 | 7.394253 |
| 283PB | 283PB Strain 10 | LGP2139 | 8.755959 | 5.691884 |
| 285PB | 285PB LGP2018 | LGP2018 | 12.031742 | 100 |
| 285PB | 285PB Strain 1 | LGP2173 | 11.21333 | 84.0138457 |
| 285PB | 285PB Strain 2 | LGP2172 | 10.228408 | 64.7752232 |
| 285PB | 285PB Strain 3 | LGP2164 | 9.964949 | 59.6290516 |
| 285PB | 285PB Strain 4 | LGP2165 | 9.033842 | 41.4416163 |
| 285PB | 285PB Strain 5 | LGP2008 | 7.982016 | 20.8961413 |
| 285PB | 285PB Strain 6 | LGP2112 | 7.609441 | 13.6186008 |
| 285PB | 285PB Strain 7 | LGP2169 | 7.485808 | 11.2036581 |
| 285PB | 285PB Strain 8 | LGP2044 | 7.402148 | 9.5695127 |
| 285PB | 285PB Strain 9 | LGP 2011 | 6.922695 | 0.2042973 |
| 285PB | 285PB Strain 10 | LGP2171 | 5.864521 | −20.4651746 |
| 286PB | 286PB Strain 1 | LGP2001 | 18.47052 | 102.4019 |
| 286PB | 286PB LGP2018 | LGP2018 | 18.29094 | 100 |
| 286PB | 286PB Strain 2 | LGP2012 | 17.23022 | 85.81258 |
| 286PB | 286PB Strain 3 | LGP2000 | 17.06282 | 83.57344 |
| 286PB | 286PB Strain 4 | LGP2015 | 16.97065 | 82.34073 |
| 286PB | 286PB Strain 5 | LGP2007 | 15.82329 | 66.99432 |
| 286PB | 286PB Strain 6 | LGP2003 | 14.07074 | 43.5534 |
| 286PB | 286PB Strain 7 | LGP2010 | 14.04739 | 43.24119 |
| 286PB | 286PB Strain 8 | LGP2013 | 13.72635 | 38.9471 |
| 286PB | 286PB Strain 9 | LGP2004 | 12.51197 | 22.7044 |
| 288PB | 288PB Strain 1 | LGP2031 | 11.73032 | 115.04974 |
| 288PB | 288PB LGP2018 | LGP2018 | 10.961572 | 100 |
| 288PB | 288PB Strain 2 | LGP2030 | 10.823393 | 97.29486 |
| 288PB | 288PB Strain 3 | LGP2184 | 10.428576 | 89.56555 |
| 288PB | 288PB Strain 4 | LGP2188 | 10.060309 | 82.35601 |
| 288PB | 288PB Strain 5 | LGP2132 | 10.004185 | 81.25727 |
| 288PB | 288PB Strain 6 | LGP2179 | 9.603427 | 73.41165 |
| 288PB | 288PB Strain 7 | LGP2183 | 9.371095 | 68.86329 |
| 288PB | 288PB Strain 8 | LGP2122 | 8.820766 | 58.08953 |
| 288PB | 288PB Strain 9 | LGP2009 | 7.664263 | 35.44871 |
| 288PB | 288PB Strain 10 | LGP2088 | 6.600541 | 14.62428 |
| 289PB | 289PB Strain 1 | LGP2002 | 16.64733 | 117.25169 |
| 289PB | 289PB LGP2018 | LGP2018 | 15.73919 | 100 |
| 289PB | 289PB Strain 2 | LGP2174 | 14.52193 | 76.87615 |
| 289PB | 289PB Strain 3 | LGP2178 | 14.47025 | 75.89433 |
| 289PB | 289PB Strain 4 | LGP2119 | 14.41787 | 74.89923 |
| 289PB | 289PB Strain 5 | LGP2070 | 14.39551 | 74.47451 |
| 289PB | 289PB Strain 6 | LGP2104 | 14.2175 | 71.09291 |
| 289PB | 289PB Strain 7 | LGP2175 | 13.17078 | 51.20856 |
| 289PB | 289PB Strain 8 | LGP2115 | 13.15135 | 50.83953 |
| 289PB | 289PB Strain 9 | LGP2177 | 13.0369 | 48.66526 |
| 289PB | 289PB Strain 10 | LGP2180 | 13.00762 | 48.10911 |

Forty-eight Methylobacterium strains were selected for gene correlation analysis from the 176 strains tested, including 15 non-hits and 33 hits. The strains were selected from those having the highest and lowest normalized root scores, excluding any isolates that had any signs of any type of microbial contamination. The normalized score standardized each isolate's mean root length value to the UTC (a value of 0) and the positive control, LGP2018 (a value of 100).

Genomes of the selected isolates were assembled and putative genes identified. The genes were assigned a putative function by sequence analysis to databases of known genes and gene signatures. A pan-genome for Methylobacterium was constructed as described by Page et al. (Roary: rapid large-scale prokaryote pan genome analysis, Bioinformatics (2015) 31:3691-3693) except that genome sequences from greater than 1000 different species of Methylobacterium were assembled and used to construct the pan-genome as opposed to the single Salmonella species described by Page et al.

The genomes of strains identified as enhancing rice seedling growth, “hits”, and strains identified as “non-hits”, were compared to determine the presence or absence in each strain of each genetic element in the pan-genome. For this analysis, translated genes were clustered across strains using BLASTP with a sequence identity of at least 50% to identify homologous genetic elements across genomes. These results were used to determine which genetic elements are the same or different across strains, leading to a score for each genetic element as present or absent in a given strain. The presence/absence scores were used in a correlation analysis to identify genetic elements that correlate positively with enhancing rice seedling growth as described by Brynildsrud et al. (Rapid scoring of genes in microbial pan-genome-wide association studies with Scoary, Genome Biology (2016) 17:238).

The steps in the process were as follows. Correlated genetic elements were collapsed so that genes that are typically inherited together, for example genes on the same plasmid, were combined into a single unit. Each genetic element in the pan-genome received a null hypothesis of no association to the trait. A Fisher's exact test was performed on each genetic element with the assumption that all strains had a random and independently distributed probability for exhibiting each state, i.e. presence or absence of the genetic element. To control spurious associations due to population structure, the pairwise comparisons algorithm was applied using a phylogenetic tree of the Methylobacterium genus, constructed using the same genome sequences described above. Empirical p-value was computed using label-switching permutations, i.e. the test statistic was generated over random permutations of the phenotype data. The genetic elements that were significantly positively correlated with enhancing rice seedling root growth were identified based on p value using a threshold for statistical significance of p less than or equal to 0.05. Sensitivity and specificity cutoffs were also employed based on the number of hits and non-hits a gene was present in.

Gene elements that were positively correlated with Methylobacterium enhancement of growth in rice seedlings are shown in Table 3 below.

TABLE 3

| | Consensus | | | | | |
|---|---|---|---|---|---|---|
| | Protein | Representative | | | | |
| Gene | SEQ ID | protein | | | | p- |
| name | NO: | sequences | Annotation | Sensitivity | Specificity | value |
| group_{—} | 17 | SEQ ID NO: 24 | hypothetical protein | 60.61 | 80.00 | 0.003 |
| 4403 | | | | | | |
| group_{—} | 18 | SEQ ID NO: 25 | hypothetical protein | 57.58 | 86.67 | 0.025 |
| 9931 | | | | | | |
| group_{—} | 19 | SEQ ID NO: 26 | hypothetical protein | 66.67 | 86.67 | 0.030 |
| 7199 | | | | | | |
| recD2_2 | 20 | SEQ ID NO: 27 | ATP-dependent RecD- | 45.45 | 93.33 | 0.035 |
| | | | like DNA helicase | | | |
| pinR | 21 | SEQ ID NO: 28 | Putative DNA- | 69.70 | 80.00 | 0.039 |
| | | | invertase from | | | |
| | | | lambdoid prophage Rac | | | |
| group_{—} | 22 | SEQ ID NO: 29 | hypothetical protein | 33.33 | 100.00 | 0.055 |
| 2780 | | | | | | |
| group_{—} | 23 | SEQ ID NO: 30 | hypothetical protein | 60.61 | 80.00 | 0.057 |
| 5546 | | | | | | |

Methylobacterium consensus protein sequences for the above identified genes that positively correlate with enhanced growth or rice seedlings are provided as SEQ ID NO: 17 through SEQ ID NO: 23 below. Consensus sequences are generated by aligning the encoded protein sequences from all isolates from a comprehensive database of Methylobacterium genome sequences from public and internal databases. EMBOSS cons was used to generate consensus sequences from the multiple sequence alignment. Where no consensus was found at a position an ‘x’ character is used. An upper case letter for an amino acid residue indicates that most of the sequences have that amino acid at that position. In the consensus sequences, X can be any amino acid residue or can be absent.

SEQ ID NO: 17
xxxxxxxxxxxxxxxxxxxxxxxxxxxxxxxxxxxxxxxxxxxxxxxxxxxxxxxxxxxxxxxMPTxLPxxxx
xxxxRxxPVRRLSWPDTARFLILVARVRLLDxxxxxxxxxxxxxxxxxxxxxxxxxxxxxxxxxxxxxxLRLH
AxxxxxxxxxxxVxRxGSxxAGDxLLxLMRRWLAxHEAIxALLPGVPEPxHVAQVxxxxxxxx
xxxxxxxxxxxxxxRAILQxxxxxxxxxVPxSRxxxxxPxxxxxxxxxxxxxxxxxxxxxxxxxxxxxxxxx
xxxxx
SEQ ID NO: 18
xxxxxxxMxxPLRRTVQVxEDGRMNLPADMRRVLGLTGAGRVILTQDEDGIxITTaEQA
LKRVRSLAAPFxRGxGSVVDEFIAERRADAAREDxExxxxxxxxxxxxxxxxxxxxxxxxxxxx
xxxxxxxxxxxxxxxxxxxxxxxxxxxxxxx
SEQ ID NO: 19
MxxxxxxxxxxxxxxxxxxxxxxxxxxxxPQSYALQILAIAxAMSVLGLGGVWIASRIYDRNTR
RLEAxxxxRRGDxxxxxxxxxxxxxxxxxxxxxxxxxxx
SEQ ID NO: 20
xxxxxxxDTLExxxxxxxxxxxxxxxxRxxxxxLACTVxDHxSIAxxQNxVPIIRDIxLxNxxDxDL
ADVxLxIxAxPxLxRPLTLxxRIxAGxxxxIDxPDLRIDxAILxxxxxAGxxESxxxxVTLxLxxS
xxxxxxxxExARExxDLRLLPPSHWGGxxAAPELLAAFVRPNDPAVDxILRxAAxILxRAx
RxTAxxDGYxSGRKARAWEMAEAIxAxxxxxAMAxxxxxxxxxxxxRIxxxxxxYVLPPASFE
RSGQKVRxPxxIVERRLxTCLDLTLLWAACxEQAGLNPLLVLTxxHAxLGLWLxDExxx
xxxxDDxQxLRKRRDLQExxxxxxxxxxxxLILIETTILTxxxxxxxxxDPPxxFxxAxxxGAxxIDx
DAxAxLEMxLDLRRxRxxGIxPLDxGExxxxxxxxAPxxxxxxxxLxxxQxLxxxxxxxxxAPPSF
xEDxxxxxIDxxxxxxPxxRLExWKxRLLDLTLRNKLLNFKPGKGSLTLDCxEPGAxEDxLx
AGxxFRLxxRPxxxxxDxxxxxxxxxxxxxxxxxxxxxxxxxxxAxxxRxEIxxxxxxxxxxxxxxxxxxE
LExRLxDLFRLARxxFEEGGANVLFLAxGFLTWTRxxGxxxxxRAPLLLVPxALxRASVR
AGFRLxxHDEExRLNPTLLEMLRQDFxLxMPDxxxxLPxDxSGIDVExIWRIVRTHIRDLK
GWEVxxEVVLSAFSFTKFLMWKDLxERxDLLKRSPVVRHLLDTPKAYGDGxxxTxFP
xPxRLDxEHPPxxIFxxxxxPLxADSSQLSAILAAASGKDFVLFGPPGTGKSxxxxxxxxxxQT
IxNMIAQCLAxxGRTVLFVSQKSAALEVVxxRRRLxxVGLGxxCLEVHAxKAQKTxVIx
QLREAWxxRxxxxxxxWDxAxxDLxxxRExLNGVVxSLHxxRxNGLSAHxAxGRVIAxxxx
GxxxxLxLxWPxxxxxxxxxxxxSLxxxxxRAxCxELxxxxxLxxxVGxIxDHPLRGIxAxxWSPL
WRxEMxxAIxxLxRTLxxxxxSGQxxAEAMGLxxLxxTYxGxxRxLxxLxxxLxRxEARxGLx
FLxxGxxxLRQAVxARxxxQxxxARLxxRLxxxYxxPxVxxxDLxxLLAEWxxAKxSNFxLRG
xRLxRVxxxLxPFAQGxxPxDIGPDLxxLxEIxxxxxxxxxxxxxxxxxxxxVxExxxAxLGxxxPxx
xxWSDPxxPAxxFxAxMAWAxRLxxVIxxMxPLxxxGxDxVRxxLxxxxxxLDxExxxLxxxxxx
xxxxxGGxLAxAxxxFxxxRxxAVKAIExLGRxxxxxxxxxLAGRAxPDxxxxPVxxExxxxxxxx
xxxxxxxxxxxxxxxxxxxxxxxxxxxxxxxxxxxxxxxxxxxxxxxxxxxxxxxxxxxxxxxxxxxxxxxxxxx
xxxxxxxxxxxxxxxxxxxxxxxxxxxxxxxxxxxxxxxDxWVxxTLAVAxRWxxxLxxKAQxWxA
WQxAAxxAxKAGLxPLVxAIExGxIxxDxxxxAFExAYARWWIDxxxTDDxxLRxxxxxFMx
QRHEEAIRxFxxADSRLSxLAxxxVRARxxxxxxxIGGGVPxxxxxxxAxAFGxDPEWGTLAx
EIxxxxxTKRxRHMPLRQLFxRMPNALTRLxxxTPCLMMSPLSIAQYxPxExKPFDIVIFDE
ASQIAPWDAIGAIARGRQVVIVGDPEQLPPTNVGDRGVDEIxxxxDGxDVADQESILDE
CLAANLPQRxLxxxxxWHYRSRHESLIAFSNxHYYxGxLVTFPSPVTDDxRAVRLxxVxD
GLYERGxxRVNRPEARALVAEVVxRLxDPxxxxxxxxxAFAxExRSLGIVTENGEQQRLIE
NLLDxERRxxxxPELExFFDxxxWxEPVFVKNLExVQGDERDAILFSVAxGPxxDxTGRxx
xxISSLNREGGHxxxRRLNVAITRARRELVVFASMRxDQVDLGRxxARGVRDFKHFLxF
AExxGAxALxxAxAPTGGDIESPFExAVMAxxxxxxxxALxARGWxIxxQVGVSxFRIDLGI
VHPDAPGRYLAGVECDGATYxxxHxAATARDRDRLRExVLTDLGWRIxRVWSTDWW
xDxQGALxRLDxxLRxDLDADRAKxxxxxxxxxxxxxxxxxxxxxxxxxxxxxxxxxxxxxxxxxxxx
xxxxxxxxxxxxxPxxxxxxxxxxxxPxxxxxxxQxxxxPxxxxxxxxxxxxxxxxxYxxADLSxxGxxxD
xxRFHDxxYxxxLAAMxAxVVxxEGPVFxDILxxRLxRAHGxxRITxxLRQxxLxxVDPxxxx
TxExxRIVLWPxGxxPxxxxxxFRPAxxxxxxxxxxRAxxxDxPLxELxGLARxLxxxxxxxxxxx
MAxRLxxxxxxxxxGLxRMxxAxxxxxxxxxxxxxxxxxxxxxxxxxxxxxxxRARFAEAxAxLxAR
EXxxxxxxxxxxxxxxxxxxxxxxxxxxxxxxxxxxxxxxxxxxxxxxxxxxxxxxxxxxxxxxxxxxxxxxxx
xxxxxxxxx
SEQ ID NO: 21
xxxxxxxxxxxxxxxxxxxxxxxxxxxxxxxxxxxxxxxxxxxxxxxxxxxxxxxxxxxxxxxxxxxxxxxxxxx
xxxxxxxxxMQTILYARVSTADQTIAHQRxQAEAAGFKIxDxVVADEGVSGVSTxLxDRP
QGRRLFDxxMLRRGDVLxxxxxxxVVRWVDRLGRNYAxxxxxxxxxxxxxxxxxxxxxxxxxx
xxxxxxxxxxDVTETIREFMRxxxxxxxRGVIVRTVINNxxxxxxxxxxMTFDGATTDPMQxA
VRDALxxxIGFMAATAQAQAEATxKEAQKAGIEHAKxRxxExDxxAYRGRKPSYTREQ
xxxDxVRxxLxQGxxxVSAIAKATGLSRQxTVYRIRDNPAEAEAALARxxxxxxxxxxxxxxx
WAAxxxxxxxxxxxxxxxxxxxxxxxx
SEQ ID NO: 22
MxxxxxxxxxxxxxxxxxxYDDxxxADAAAGEERDAIMRALAEDMxEASxxxxRxxxxxGxF
VRAERPADLxxxxxxxxxxxxxxxxxxxxxxxxxxxxxxxxxxxxxxxxxxxxxxxxxxxxxxxxxRAL
GRxxxxxDRRxxQxxxxxxxxxxxxxxxRxASxxxxxxxxxxxxxxxxxxxxxxxxxxxxxxxxxxxxxxx
SEQ ID NO: 23
xxxxxxxxxxxxxxxxxMPVxxGIGIGRGDPLRPAVTRTxRFSGPEGFHxxPGALWLAAAAP
LLATxLLLLxxRLAA
Representative amino acid sequences for proteins correlated with enhancing growth of rice
seedlings from specific Methylobacterium strains are provided below as SEQ ID NO: 24
through SEQ ID NO: 30. The strain from which a representative sequence was obtained is
referenced below.
LGP2022
SEQ ID NO: 24
MPTAIPIRPAPERCLSWPDTARLLILVARVRILDLEMHTVVRHGSGFADDRLLHLMR
RWLAQHEAISALLPGVAEPRHVAEVRAILQVPNSRPEPEDRRAL*
LGP2021
SEQ ID NO: 25
MPQRRTIQVTEDGRMNLPADIRRVLGLNGAGRIVLMQDEDGIHLTTAEDPLRRVREL
AAPFRRGSGSVVDEFIAERRADSGED*
LGP2021
SEQ ID NO: 26
MPLDYALQITAIAFGLSVLGLGGAFIASRVYDRNTRRYDEAAQLHKAD*
LGP2021
SEQ ID NO: 27
VQDGIQITCSVTEHVSLAYHENAVPVIREVVVENTSEQELSDVRVRIESRPAVVQPLT
LRIDRIPAGSNHHIELPDVRLDAALLAGFTEASRLELTVIVEDAAGERARHLEELRVLP
PSHWGGGRSAPELLAAFVRPNDPAVDVVLRDAATKLGEAGRETGLNGYTTAKKSR
AWELAEAIWAAIADRRIAYVLPPASFERAGQKVRGPSDVLERKVGTCLDLSLLYAAC
LEQAGLNPVLVLTVGHAFVGVWLQDDDFASATVDDMQLLRKRRDLQDLVFVETTL
LTPEPPATFKVATTQGGVQVEDEAPAALEIAIDVRRCRRRGIRPMDLGDGKPTGIAPA
PTIPLNQTLSAPPSFEEEARAPVDEAPETPVGRVERWKRKLLDLTLRNKLLNFKPGKG
SVSLECASPGALEDGLAAGTEYRLKPLSDVLTGSDERSADLYARRHHDDGRRSYLE
AALARKEIYTTSTEADLDRRLLDLYRLARNGFEEGGANILFLAVGELSWTKKEGEAA
YRAPLLLVPVTLKRSSVRAGFKLALHDDEVRINPTLLEMLREDFKLRMPELEGDLPR
DGSGYDVDGIFRIVRQHVKELRGWEVVPDVVLSAFSFTKYLMWKDLVDRAEVLKR
NPVVRHLIDTPKHSYGDGTPFPEPTRLDREHPPETVFAPLSADSSQLSAVLAAAGGKD
FVLFGPPGTGKSQTIGNMIAQCLAQGRTVLFVSQKTAALEVVQRRLQEIGLGDYCLE
VHSTKAQKSAVLGQLRRAWHERSTPSQGTWDAATSELASLREELNGLVNALHRRRE
NGLSAYEAFGRVIASGGEAPLVLTWPDHLAHNETTLANLRAACRELRPVLASVGSL
VDHPLQGVEATQWSPVWRDDMGAAIRAVEQTLGALRVSGQAFAEAIGLPSLLATY
AGTRGLVVLGNYLVRSEARCGAAFLADGAGDLRRAVAARERFQTTKVQLLGRLTG
RYRPGILDQNLGALLAEWVAAQGANFLVKGGKLKKVSAQVQPYAEGPLPPDLGPD
LTGLIEVARHVKAGCLEELILARLGLPWSNPDCPASEFASAITWAEKVEQLLDILGPL
SLGIDGLRDHLVHLVERQGRALADGGRIAQTYAAFAQDRARANEAMKALGVLAGR
PDPEEPLAAEADWIERSCTIARRLSSGLSRAQGWCAWQAAAQSALKTGLAPLIDALE
DGRIAPDRAEIAFEIAYARWWIDRVVSDDPVLRRFLPARHEDAIQRFRAADARVTEL
SKQVVRSRLGGGIPGATAFGADPEWGTLSHELTKKTAHMPLRKLFGKMPTALTKLT
PCVMMSPLSIAQYLPPDKEPFDVVIFDEASQISPWDAIGALARAKQVVIVGDPEQLPP
TNVGDRGVDDIEDGSDVTDQESILDECLAANIPRRNLDWHYRSRHESLIAFSNSRYY
GGRLVTFPSPVTDDRAVRLTLVPDGVYKRGSGRVNRPEARAVVADIVRRLRDPSFSE
ERRSLGVVTFNGEQQRLIENLLDEQRRSYPELEPFFDRDRWHEPVFVKNLENVQGDE
RDAIIFSVAVGPDQTGRPVSTVSSLNKDGGHRRLNVAITRARRELVVFASMRPEQIDL
GRTRARGVRDFKHFLEFAERGARALAEAFAPTGGDVESPFEAAVMAGLEARGWTV
HTQIGVSGFRIDLGIVHPDAPGRYLAGVECDGATYHSSATARDRDRLREHVLTDLG
WRIRRVWSTEWWMDAEGALTKLDQRLIEDLEADRAKAAAAAAEAPRDVAVEPEA
VEQEHDEPTGEPEVTPPVDTGPSEPANDLEPVTDLIPQRLYADQALPVTPPAPKPEVY
DDVRAYRIVDLNDLGRSVEPGRFYDASYQQALSAMVDHVLAVEGPIYEELLIKRIAR
AHDIQRVGPLVREAIADRIDASVARTEDDGRPVLWPRGEEPRASYPHRPASAAIRSHT
DTPMPELVGIAMTLPSNASEAERARMIGQRLGLSRIEASARARFERASELARQAAVA*
LGP2022
SEQ ID NO: 28
MSVVLYARVSTAEQTLEHQQTQAEAAGFVFDAVVADHGESGRKPLRDRPEGRRLY
DMLRTGDVLVVRWINRLGRSYEDVTGVMRELMQRGVIVRTIISNMTFDGATKDPM
QRAIRDALIAFMAAAGEAELEATREAQKAGIEHARKQADQTAYRGRKPSYTRDQLT
VISGMLGRGAGVSAIAAETGLSRQTIYRVQADPVEAEAALARWA*
LGP2016
SEQ ID NO: 29
MLSLDDIAAAAAGEERDALWRSLVEDMEEAAGRRRGGRGLVQADRPADLARALGR
DRRVQPSRLARSAS*
LGP2022
SEQ ID NO: 30
MPVGIGIGRGDPLRPAVTRTARFSGPEGFHPGALWLAAASPLLATLLLLVRLAA*

### Example 2 Methylobacterium Inoculation Effect on Nitrogen Utilization in Rice

Methylobacterium isolates were tested for their ability to enhance shoot nitrogen content and/or concentration in rice. A randomized complete block design was used, with 12 treatments in each run; five Methylobacterium isolates and a control at two nitrogen levels. The untreated control sample (UTC) was Methylobacterium growth medium applied in the same amount as used for the Methylobacterium isolates. Each treatment level had an n of 10. All 10 blocks were grown in the same growth chamber and on the same shelf.

### Procedure:

### Media:

0.5× Murashige and Skoog MS medium with high or low nitrogen
   High nitrogen media—10400 uM
   Low nitrogen media—250 uM

### Pre-Planting:

Rice seeds were de-husked. Average 100 seed count is 2018 mg with approximately 21 g of husked rice per run.
Agar plates containing high or low nitrogen media were prepared.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least on hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants were measured as follows.
Plants that were not impeded from growing normally (by physical surroundings unrelated to presence of Methylobacterium) were removed from plates, and the number of seedlings for that plate recorded.
Seedlings were scanned using WinRhizo and the images analyzed to determine root and shoot area for each plant.
Seedlings were rinsed to remove any remaining plate media and the shoots separated from the seedlings and dried in a drying oven for at least 3 days.
Dried shoots were combined for each treatment and the mass measured. The plant material was then ground to a powder to be used for nitrogen testing.
Nitrogen analysis was conducted on the powdered samples by Atlantic Microlab (Norcross, GA).

Results of the analyses are shown below. In all tables, pairwise results are presented separately for the High N and Low N treatments. Data was analyzed using Student's t-test and different letters indicate a significant difference between treatments at p<0.05.

TABLE 4
Exp 1
Shoot Area Measurements

| 4A Low Nitrogen Treatment | | | 4B High Nitrogen Treatment | | |
|---|---|---|---|---|---|
| | | Mean Shoot Area | | | Mean Shoot Area |
| Treatment | | per Plant (cm²) | Treatment | | per Plant (cm²) |
| LGP2033 | A | 0.30 | LGP2020 | A | 0.51 |
| UTC | A | 0.30 | LGP2033 | B | 0.42 |
| LGP2009 | A | 0.29 | LGP2022 | BC | 0.40 |
| LGP2020 | A | 0.29 | LGP2003 | BC | 0.40 |
| LGP2022 | A | 0.28 | UTC | BC | 0.36 |
| LGP2003 | A | 0.28 | LGP2009 | C | 0.34 |

TABLE 5
Exp 1
Root Area Measurements

| 5A Low Nitrogen Treatment | | | 5B High Nitrogen Treatment | | |
|---|---|---|---|---|---|
| | | Mean Root Area | | | Mean Root Area |
| Treatment | | per Plant (cm²) | Treatment | | per Plant (cm²) |
| LGP2020 | A | 0.93 | LGP2020 | A | 0.99 |
| LGP2022 | A | 0.88 | LGP2022 | B | 0.85 |
| LGP2033 | AB | 0.85 | LGP2033 | B | 0.83 |
| LGP2009 | B | 0.79 | LGP2003 | C | 0.67 |
| LGP2003 | B | 0.77 | LGP2009 | C | 0.62 |
| UTC | C | 0.64 | UTC | C | 0.59 |

TABLE 6
Exp 1
Shoot Nitrogen Concentration

| 6A Low Nitrogen Treatment | | | 6B High Nitrogen Treatment | | |
|---|---|---|---|---|---|
| | | Mean % Dry Wt | | | Mean % Dry Wt |
| Treatment | | Nitrogen | Treatment | | Nitrogen |
| UTC | A | 2.73 | LGP2020 | A | 4.92 |
| LGP2020 | B | 2.59 | LGP2022 | B | 4.38 |
| LGP2022 | C | 2.48 | LGP2033 | C | 4.02 |
| LGP2033 | C | 2.49 | UTC | D | 3.23 |
| LGP2009 | D | 2.35 | LGP2009 | D | 3.27 |
| LGP2003 | D | 2.30 | LGP2003 | D | 3.26 |

Significant and substantial shoot growth promotion was observed for some isolates at high nitrogen. Shoot growth promotion was not observed for the Methylobacterium treatments at low nitrogen, consistent with some literature reports which indicate that growth promotion effects from plant-beneficial microbes may not be observed when nutrient availability is too low. Root growth promotion was evident at both nitrogen levels and Root/Shoot ratios are higher under low N than under high N. As expected, plants grown on high N media showed substantially greater shoot N concentration than those grown on low N media. Several Methylobacterium isolates demonstrated significantly enhanced shoot nitrogen concentration under high nitrogen growth conditions. Three isolates, LGP2020, LGP2022, and LGP2033, demonstrated the greatest enhancements of shoot growth, root growth and shoot nitrogen concentration.

The above experiment was repeated using four of the same Methylobacterium isolates and one additional isolate. Results were similar to those observed in the first assay and are shown in the tables below. LGP2020 (NRRL-B-67892), LGP2022 (NRRL-B-68033), and LGP2033, again demonstrated enhancements of shoot growth, root growth and shoot nitrogen concentration.

TABLE 7
Exp 2
Shoot Area Measurements

| 7A Low Nitrogen Treatment | | | 7B High Nitrogen Treatment | | |
|---|---|---|---|---|---|
| | | Mean Shoot Area | | | Mean Shoot Area |
| Treatment | | per Plant (cm²) | Treatment | | per Plant (cm²) |
| LGP2022 | A | 0.18 | LGP2022 | A | 0.30 |
| LGP2033 | A | 0.19 | LGP2033 | AB | 0.30 |
| LGP2020 | A | 0.17 | LGP2020 | AB | 0.29 |
| UTC | A | 0.19 | UTC | AB | 0.26 |
| LGP2003 | A | 0.18 | LGP2003 | AB | 0.25 |
| LGP2019 | A | 0.18 | LGP2019 | B | 0.25 |

TABLE 8
Exp 2
Root Area Measurements

| 8A Low Nitrogen Treatment | | | 8B High Nitrogen Treatment | | |
|---|---|---|---|---|---|
| | | Mean Root Area | | | Mean Root Area |
| Treatment | | per Plant (cm²) | Treatment | | per Plant (cm²) |
| LGP2033 | AB | 0.57 | LGP2033 | A | 0.67 |
| LGP2022 | AB | 0.53 | LGP2022 | A | 0.66 |
| LGP2020 | A | 0.59 | LGP2020 | A | 0.64 |
| LGP2019 | AB | 0.56 | LGP2019 | B | 0.54 |
| LGP2003 | AB | 0.52 | LGP2003 | B | 0.49 |
| UTC | B | 0.50 | UTC | B | 0.47 |

TABLE 9
Exp 2
Shoot Nitrogen Concentration

| 9A Low Nitrogen Treatment | | | 9B High Nitrogen Treatment | | |
|---|---|---|---|---|---|
| | | Mean % Dry Wt | | | Mean % Dry Wt |
| Treatment | | Nitrogen | Treatment | | Nitrogen |
| LGP2020 | AB | 2.36 | LGP2020 | A | 4.28 |
| LGP2022 | AB | 2.30 | LGP2022 | A | 4.06 |
| LGP2033 | AB | 2.38 | LGP2033 | B | 3.68 |
| UTC | A | 2.51 | UTC | BC | 3.45 |
| LGP2003 | B | 2.25 | LGP2003 | C | 3.37 |
| LGP2019 | B | 2.21 | LGP2019 | C | 3.23 |

Percent difference between Methylobacterium treatments and UTC at high and low N for 3 different variables: projected root area, projected shoot area and foliar nitrogen concentration, are shown for each experiment. Bold italics are used to denote a statistically significant difference from UTC at p<0.05 using Student's M-est.

TABLE 10
Percent Differences

| | | | | | | % N | % N |
|---|---|---|---|---|---|---|---|
| N | | % Root | % Root | % Shoot GP | % Shoot GP | Enhancement | Enhancement |
| Level | Treatment | GP Exp 1 | GP Exp 2 | Exp 1 | Exp 2 | Exp 1 | Exp 2 |
| High | LGP2003 | +15.1% | +2.8% | +10.6% | −1.7% | −0.8% | −2.2% |
| N | LGP2020 | +68.5% | +35.0% | +42.0% | +14.0% | +49.7% | +23.9% |
| | LGP2033 | +41.6% | +42.2% | +16.2% | +15.5% | +22.4% | +6.8% |
| | LGP2022 | +45.4% | +40.1% | +10.8% | +15.8% | +33.3% | +17.7% |
| Low | LGP2003 | +19.4% | +4.5% | −8.9% | −8.6% | −15.8% | −10.2% |
| N | LGP2020 | +43.5% | +18.3% | −3.2% | −11.5% | −5.3% | −6.1% |
| | LGP2033 | +31.8% | +13.8% | +0.7% | −2.5% | −9.1% | −5.0% |
| | LGP2022 | +37.0% | +6.1% | −8.6% | −8.5% | −9.0% | −8.3% |

### Example 3 Evaluation of Optimal Nitrogen Dose for Testing Methylobacterium Effect

The high nitrogen dose in the experiments described above is the amount in 0.5× MS media, a general plant growth medium, and provides the optimal amount of nitrogen for plant growth. To evaluate plant response to Methylobacterium treatment under various reduced nitrogen levels, including a nitrogen level that approximates the amount of nitrogen in a field treated with a 25-30% reduction of optimal nitrogen level, two low nitrogen dose experiments were conducted.

Nitrogen doses used for evaluation of effect of Methylobacterium treatment on plant growth were: 5200 uM nitrogen (5000 of rice optimal nitrogen level), 7280 uM nitrogen (700% of rice optimal nitrogen level) and 10400 uM nitrogen (100% of rice optimal nitrogen level). Results are shown in Tables 11-13 below. Data was analyzed using Student's t-test and different letters indicate a significant difference between treatments at p<0.05.

TABLE 11
Exp 3
Shoot Area Measurements

| | 5200 μM N | | 7280 μM N | | 10400 μM N | |
|---|---|---|---|---|---|---|
| | Treatment Mean | | Treatment Mean | | Treatment Mean | |
| | Shoot Area | | Shoot Area | | Shoot Area | |
| Treatment | per Plant (cm²) | | per Plant (cm²) | | per Plant (cm²) | |
| LGP2020 | A | 0.41 | A | 0.36 | A | 0.41 |
| LGP2033 | B | 0.33 | A | 0.34 | B | 0.34 |
| Control | C | 0.28 | B | 0.25 | BC | 0.30 |
| LGP2019 | C | 0.27 | B | 0.28 | C | 0.28 |

TABLE 12
Exp 3
Root Area Measurements

| | 5200 μM N | | 7280 μM N | | 10400 μM N | |
|---|---|---|---|---|---|---|
| | Treatment Mean | | Treatment Mean | | Treatment Mean | |
| | Root Area | | Root Area | | Root Area | |
| Treatment | per Plant (cm²) | | per Plant (cm²) | | per Plant (cm²) | |
| LGP2020 | A | 0.82 | A | 0.78 | A | 0.79 |
| LGP2033 | B | 0.70 | A | 0.77 | B | 0.71 |
| LGP2019 | B | 0.62 | B | 0.64 | C | 0.57 |
| Control | C | 0.47 | C | 0.45 | D | 0.49 |

TABLE 13
Exp 3
Shoot Nitrogen Concentration

| | | | |
|---|---|---|---|
| | 5200 μM N | 7280 μM N | 10400 μM N |
| | Treatment | Treatment | Treatment |
| | Mean % Dry | Mean % Dry | Mean % Dry |
| Treatment | Wt Nitrogen | Wt Nitrogen | Wt Nitrogen |

| | | | | | | |
|---|---|---|---|---|---|---|
| LGP2020 | A | 4.70 | A | 4.40 | A | 4.61 |
| LGP2033 | B | 3.77 | B | 4.02 | B | 3.96 |
| LGP2019 | C | 3.14 | C | 3.42 | C | 3.41 |
| Control | C | 3.13 | C | 3.22 | C | 3.34 |

Nitrogen doses used for evaluation of effect of Methylobacterium treatment on plant growth were: 1560 uM nitrogen (15% of rice optimal nitrogen level), 2600 uM nitrogen (25% of rice optimal nitrogen level) and 5200 uM nitrogen. (50% of rice optimal nitrogen level). Results are shown in Tables 14-16 below.

TABLE 14
Exp 4
Shoot Area Measurements

| | 1560 μM N | | 2600 μM N | | 5200 μM N | |
|---|---|---|---|---|---|---|
| | Treatment Mean | | Treatment Mean | | Treatment Mean | |
| | Shoot Area | | Shoot Area | | Shoot Area | |
| Treatment | per Plant (cm²) | | per Plant (cm²) | | per Plant (cm²) | |
| LGP2020 | A | 0.28 | A | 0.32 | A | 0.38 |
| LGP2017 | A | 0.27 | AB | 0.28 | AB | 0.31 |
| LGP2019 | AB | 0.26 | B | 0.26 | B | 0.26 |
| Control | B | 0.23 | C | 0.22 | B | 0.25 |

TABLE 15
Exp 4
Root Area Measurements

| | 1560 μM N | | 2600 μM N | | 5200 μM N | |
|---|---|---|---|---|---|---|
| | Treatment Mean | | Treatment Mean | | Treatment Mean | |
| | Root Area | | Root Area | | Root Area | |
| Treatment | per Plant (cm²) | | per Plant (cm²) | | per Plant (cm²) | |
| LGP2020 | A | 0.75 | A | 0.73 | A | 0.71 |
| LGP2017 | AB | 0.72 | B | 0.65 | AB | 0.66 |
| LGP2019 | B | 0.65 | B | 0.63 | B | 0.61 |
| Control | C | 0.45 | C | 0.44 | C | 0.45 |

TABLE 16
Exp 4
Shoot Nitrogen Concentration

| | | | |
|---|---|---|---|
| | 1560 μM N | 2600 μM N | 5200 μM N |
| | Treatment | Treatment | Treatment |
| | Mean % Dry | Mean % Dry | Mean % Dry |
| Treatment | Wt Nitrogen | Wt Nitrogen | Wt Nitrogen |

| | | | | | | |
|---|---|---|---|---|---|---|
| LGP2020 | A | 3.03 | A | 3.65 | A | 4.67 |
| LGP2017 | A | 3.00 | B | 3.51 | B | 4.22 |
| LGP2019 | AB | 2.86 | C | 3.30 | C | 3.25 |
| Control | B | 2.73 | D | 2.90 | C | 3.15 |

Results again demonstrate significant and substantial shoot and root growth promotion and increased levels of shoot nitrogen levels resulting from treatment with Methylobacterium isolates. Shoot area correlated closely to nitrogen levels measured in shoots. Although root area measurements were not observed to be in proportion to increased nitrogen uptake as measured in shoots, additional observations noted that numbers of root tips were increased in line with enhanced nitrogen uptake as measured in shoot nitrogen concentration.

Experiments to identify additional Methylobacterium strains that can enhance plant growth and development under reduced nitrogen levels are conducted using a 7280 μM nitrogen treatment, representing 70% of the optimal N level for rice, or a 30% reduction in nitrogen fertilizer application for rice cultivation. Analysis of NLS0693 in this manner demonstrated that this strain resulted in increased shoot nitrogen concentration and increased shoot area in the rice plate assay when the concentration of nitrogen in the media is 70% of the standard nitrogen concentration.

### Example 4. Increases in Rice Yield by Application of Methylobacterium

Rice field trials were conducted at three locations, all near Humphrey, AR, for the purpose of evaluating the effects of three Methylobacterium isolates applied as a seed treatment. Treatments included each Methylobacterium isolate and an untreated control applied to rice seeds with and without a base treatment of insecticide only (active ingredient Clothiandin). The trial was conducted using a Randomized Complete Block Design (RCBD) with 4 reps per location. LGP2016 (NRRL B-67341), LGP2019 (NRRL B-67743) and LGP2017 (NRRL B-67741) were applied to rice seeds at a target concentration of 10⁶ CFU/seed.

The Methylobacterium isolates increased yield in rice field trials as compared to the untreated control both with and without insecticide treatment as shown in the Table below.

TABLE 17
Mean yield (Bu/A) Increase over control and percent
increase shown (Bold italics indicates a significant
difference at p < 0.05 using Fisher's LSD test.)

| Treatment | UTC | LGP2016 | | LGP2019 | | LGP2017 | |
|---|---|---|---|---|---|---|---|
| Without | 143.8 | 150.1 | +6.3 | 156.2 | +12.4 | 152.4 | +8.6 |
| insecticide | | | (4.3%) | | (8.6%) | | (6.0%) |
| treatment | | | | | | | |
| With | 151.8 | 164.3 | +12.5 | 155.4 | +3.6 | 158.2 | +6.4 |
| insecticide | | | (8.2%) | | (2.4%) | | (4.2%) |
| treatment | | | | | | | |

Also provided herein are methods of improving growth and yield of rice plants by treating rice plants, plant parts or seeds with one or more Methylobacterium isolates. In some embodiments, harvested seed yield and/or nutrient content of rice plants is improved. In some embodiments, rice seeds are treated and such treatment provides for increased rice seed yield. In some embodiments, the Methylobacterium isolate is selected from the group consisting of LGP2016 (NRRL B-67341), LGP2017 (NRRL B-67741), LGP2019 (NRRL B-67743) and variants of these isolates. Rice plants, plant parts or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2016, LGP2017, or LGP2019

### Example 5. Procedure to Test Methylobacterium Inoculation Effect on Nitrogen Utilization in Rice

Additional Methylobacterium strains, including Methylobacterium strains that caused increased root length during early rice growth from Example 1, are tested for Methylobacterium inoculation effect on nitrogen utilization in rice.

The experiment is conducted replacing the high and low nitrogen conditions with using 7280 uM nitrogen (70% of rice optimal nitrogen level). Data can be analyzed using Student's t-test to determine significant differences between strains at p<0.05 to determine strains that have increased nitrogen uptake compared to untreated control samples.

Results shown in Table 18 below provide percent differences in foliar N concentration in treated rice plants compared to N levels in untreated seedlings. Foliar tissue was harvested, dried, and assayed for nitrogen concentration via elemental combustion analysis.

TABLE 18

| Methylobacterium | Percent difference from Untreated in | Number of |
|---|---|---|
| Strain | Foliar N concentration (% by mass) | times tested |
| LGP2020 | +45.2% | 9 |
| LGP2023 | +47.6% | 1 |
| LGP2031 | +38.2% | 3 |
| LGP2034 | +43.9% | 1 |
| LGP2029 | +35.7% | 3 |
| LGP2021 | +41.0% | 1 |
| LGP2167 | +40.5% | 1 |
| LGP2030 | +32.0% | 3 |
| LGP2002 | +42.8% | 1 |
| LGP2018 | +37.5% | 1 |
| LGP2001 | +29.2% | 1 |
| LGP2015 | +27.9% | 1 |
| LGP2188 | +3.0% | 1 |
| LGP2189 | −4.8% | 1 |
| LGP2005 | −4.9% | 1 |
| LGP2004 | −4.7% | 1 |
| LGP2032 | +30.0% | 1 |
| LGP2024 | +31.3% | 1 |
| NLS0681 | +27.2% | 1 |
| NLS0594 | +24.2% | 1 |
| NLS0479 | +43.2% | 1 |
| NLS1310 | +44.2% | 1 |
| NLS0612 | +38.1% | 1 |
| NLS1312 | +36.5% | 1 |
| NLS0473 | +32.6% | 1 |
| NLS0706 | +34.5% | 1 |
| NLS0725 | +34.9% | 1 |
| NLS0159 | +5.1% | 1 |
| NLS0229 | −1.5% | 1 |

### Example 6: Growth of Methylobacterium Isolates Containing Methane Monooxygenase (sMMO) Genes Using Methane as Sole Carbon Source

Methylobacterium strains and positive and negative controls are grown on ammonium mineral salts (AMS) media plates, and serial dilutions conducted to determine the appropriate dilution for a target range of 30-300 colonies per plate. For the initial sample tube, 20 ml of 0.9% saline was added, and the mixtures was vortexed for 5 minutes individually using a standard test tube adaptor or up to 6 at a time using a horizontal tube adaptor (SI-V506 for vertical holder). A 1:10 dilution series was created from the initial tube (10E0) to 10E−6. The first time a sample was analyzed, all dilutions were plated to identify the target range of 30-300 colonies per plate. A pure Methylobacterium positive control sample was plated so there would be 50-100 colonies per plate.

After completion of the dilution series, the appropriate dilutions were plated onto AMS agar plates in triplicate, and a spreader was used to spread the cells around the plate. A new sterile plastic spreader was used for each dilution or a glass spreader was flamed between dilutions. When finished plates were placed upside down in the acrylic vacuum chamber_A vacuum was applied to create a partial vacuum in the gas-tight vessel (typically −15 psig). High-purity methane (99.999%) was added to create an internal vessel pressure of 0 psig. This creates a methane:air ratio of ˜1:2.

After 10 days the number of colony forming units per sample was counted and recorded. Any plates that have no colonies were placed back in the incubator and checked at 14 days and then 2 days if necessary. Growth of Methylobacterium strains containing sMMO genes and positive control strains is observed, whereas no growth is observed on negative control plates.

### Example 7: Sequences of sMMO Protein Components and Genes Encoding Same

Sequences of genes from representative Methylobacterium strains that encode sMMO protein components and the encoded protein sequences are provided in the accompanying sequence listing as noted in the Tables below.

TABLE 19

| Strains | SEQ ID NO | sMMO Component |
|---|---|---|
| NLS0737 | 1 | Regulatory B component amino |
| NLS0770 | | acid sequence |
| NLS0737 | 2 | Hydroxylase beta chain amino |
| NLS0770 | | acid sequence |
| NLS0737 | 3 | Reductase C component amino |
| NLS0770 | | acid sequence |
| NLS0737 | 4 | Hydroxylase alpha chain amino |
| NLS0770 | | acid sequence |
| NLS5278 | 5 | Regulatory B component amino |
| NLS5334 | | acid sequence |
| NLS5480 | | |
| NLS5549 | | |
| NLS5278 | 6 | Hydroxylase beta chain amino |
| NLS5334 | | acid sequence |
| NLS5480 | | |
| NLS5549 | | |
| NLS5278 | 7 | Reductase C component amino |
| NLS5334 | | acid sequence |
| NLS5480 | | |
| NLS5549 | | |
| NLS5278 | 8 | Hydroxylase alpha chain amino |
| NLS5334 | | acid sequence |
| NLS5480 | | |
| NLS5549 | | |

TABLE 20

| Strains | SEQ ID NO | sMMO Component |
|---|---|---|
| NLS0737 | 9 | Regulatory B component nucleotide |
| NLS0770 | | encoding sequence |
| NLS0737 | 10 | Hydroxylase beta chain nucleotide |
| NLS0770 | | encoding sequence |
| NLS0737 | 11 | Reductase C component nucleotide |
| NLS0770 | | encoding sequence |
| NLS0737 | 12 | Hydroxylase alpha chain nucleotide |
| NLS0770 | | encoding sequence |
| NLS5278 | 13 | Regulatory B component nucleotide |
| NLS5334 | | encoding sequence |
| NLS5480 | | |
| NLS5549 | | |
| NLS5278 | 14 | Hydroxylase beta chain nucleotide |
| NLS5334 | | encoding sequence |
| NLS5480 | | |
| NLS5549 | | |
| NLS5278 | 15 | Reductase C component nucleotide |
| NLS5334 | | encoding sequence |
| NLS5480 | | |
| NLS5549 | | |
| NLS5278 | 16 | Hydroxylase alpha chain nucleotide |
| NLS5334 | | encoding sequence |
| NLS5480 | | |
| NLS5549 | | |

### Example 8 Rice Field Evaluation

Mitigation of methane (CH₄) emission from the rice crop-soil system was evaluated following the application of NLS0737 and NLS0770 to rice seeds and evaluation of methane levels during the crop season. Two sites near Ita Ibate and Mercedes were used in the testing program. Both locations were in the main rice growing areas in the Corrientes province of Argentina. The plots were installed and cultivated using conventional rice farming operations. Trial layouts are provided below.

TABLE 21
Macro plots with replicates. RCBD with
5 true replicates. 2 locations.

| | Individual plot | | | | | | Area/ |
|---|---|---|---|---|---|---|---|
| | Width | Length | Area | | | Plots/ | location |
| Location | m | m | m² | Reps | # Trt | location | m² |
| Ita Ibate | 5.78* | 20 | 115.6 | 5 | 7 | 35 | 4046 |
| Mercedes | 1.95** | 15 | 29.25 | 5 | 7 | 35 | 1024 |

*33 rows × 17.5 cm
**11 rows × 17.5 cm

TABLE 22
Treatments: Yield + Methane

| Trt | Name | Rate | Target |
|---|---|---|---|
| T1 | Untreated control | | Control |
| T2 | Rizoderma | 200 mL Premax T + | Control |
| | | 600 mL Rizoderma | |
| T3 | NLS0737.05 | 62 g/100 kg | Methane |
| T4 | NLS0770.05 | 62 g/100 kg | Methane |

### Seed Treatment and Planting Process

Rice seeds were treated in rotating drums in small batches. A photographic record of the process and final seed appearance for each treated batch were collected. Seed was treated with base fungicide+insecticide for all treatments ((Acronis (BASF)−thiophanate methyl 36.9%+pyraclostrobin 4.1% or Thiram+Carbendazim+Imacloprid). NLS0737 and NLS0770 were applied at a rate of 62.5 g in 600 ml of water/100 kg of seed for a target of 10⁶ CFU per seed. Seed was enumerated for CFU of viable PPFMs and planted within seven days of seed treatment. Both locations were planted using conventional methods on a commonly farmed varietal, IRGA 424 RI seed, at 100 Kg/ha.

Fertilizer was applied pre-plant broadcast using 60-100 kg KCl, and 100 kg/ha MAP at planting, in the seed row. Urea was applied pre-irrigation at 100 kg/ha and post irrigation at 50 kg/ha during the spike differentiation stage.

Untreated control: includes base chemical fungicide/insecticide treatments following farmer standards. Seed included professional seed treatment—all biological treatments were added as over treatments. At Mercedes seed was treated with Thiram+Carbendazim+Imidacloprid. At Ita Ibate the seed was treated with Acronis (BASF)−thiophanate methyl 36.9%+pyraclostrobin 4.1%.

Commercial control: included a biostimulant/biofertilizer treatment on top of the base chemical treatment. Rizoderma (Trichoderma harzianum) was applied per recommended label rate.

### Foliar Spray:

Additional treatments were applied as foliar applications using NLS0737 and NLS0770. Conventional backpack spray technologies with a 2 meter boom were used to deliver 125 g/acre of the dried powder inoculant (˜10⁹ CFU per gram) in water at nine gallons/acre and ˜20 psig. Applications were made three times throughout the growing season at approximately 15, 35 and 50 days after sowing. Plots were split to allow randomized complete block analysis.

### Pre-Plant Soil Sampling and Crop Measurements

Soil was analyzed (0-20 centimeteres depth) to determine % organic matter, % total nitrogen, NO₃, NH₄, pH, complete macro-micro nutrient analysis and cations+EC, and soil texture.

Crop measurements included early stand count at four and 20-days post first observed emergence in the field. Plant diseases throughout all crop stages were scouted using quantitative incidence and severity scales. Digital images for NDVI and other spectral indices for visual and quantitative assessment of treatment effects were collected using two drone flights at vegetative and reproductive stages with multispectral sensors. Weekly satellite images were analyzed from Planetscope satellite imagery at 3-m resolution for NDVI time series. GPS coordinates at each corner of trial polygons were used to digitize data and analyze statistical comparisons of treatment effects through different spectral indices associated with crop growth and health. Digital elevation models were included in the two field-scale trials to assess elevation effect on crop performance. Grain yield at plot and sub-strip scale were collected by hand. Grain samples containing one kilogram from each block were assed for grain quality: % whole grain, % broken, and % chalky grain. Complete daily weather information and irrigation scheduling and amounts were recorded.

### Methane Measurements:

Greenhouse gas collections methods were designed and contracted with specialists from The USDA ARS and executed by scientists under contract from EEA. INTA. Balcarce (National Institute of Agricultural Technology, Balcarce Agricultural Experimental Station). Gas samples were collected using standard methods recognized by the ICCP following standard GC protocols. Headspace gas samples were analyzed at using dedicated gas chromatography methods on an Agilent Gas Chromatotraphy system. Cylinder head space samples were collected between 9 am and 11 am. There were 5 samples collected per plot at 15-20-min intervals. A total of 100 samples were collected per time point, from 20 chambers installed per field. Measurements were taken six times during the crop season:
1. Early tillering. 5 days after first irrigation. approx. 20 days after emergence—expected peak
2. Mid tillering: approx. 35 days after emergence
3. Late tillering: 50 days after emergence
4. Pre flowering: 65 days after emergence
5. Flowering: 80 days after emergence—expected peak
6. Advanced grain filling (pre-maturity): 100 days after emergence

Under paddy rice conditions peak methane emissions are generally recognized between time points 5 and 6. Response variables include Methane (CH₄), Nitrous oxide (N₂O) and Carbon Dioxide (CO₂), expressed in kg/ha/day. Using the six measurements across the season, a model was fit to estimate total emission in kg/ha during the entire crop cycle.

### Methane Emission Results

Methane emission rates are shown in the tables below. Peak emission occurred between late tillering and flowering, as expected. Total emissions were strongly influenced by temperature fluctuations. Overall lower emission at ItaIbate vs Mercedes were most likely related to later planting which led to overall lower crop growth, both below and above ground. Average lower temperature and radiation during CH₄ measurements combined with significant soil texture differences (Italbate sandier) also contributed to the difference in both methane emissions and reduced yield.

NLS0737 and NLS0770 showed reductions in CH₄ emissions during peak rice growth at both locations in Argentina. At the Mercedes site NLS0737 reduced methane emissions 28% (151 kg/ha) and NLS0770 reduced emissions 23% (125 kg/ha). At the Ita Ibate site NLS0737 reduced methane emissions 7% (19 kg/ha) and NLS0770 reduced emissions 4% (11 kg/ha).

TABLE 23

| Ita Ibate | % | Kg/HA | Reduction (Kg/HA) |
|---|---|---|---|
| NLS0737 | 7.0 | 251 | 19 |
| NLS0770 | 4.1 | 259 | 11 |
| Avg. | 5.6 | 270 | 15 |

| Mercedes | % | Kg/HA | Reduction (Kg/HA) |
|---|---|---|---|
| NLS0737 | 28.1 | 386 | 151 |
| NLS0770 | 23.3 | 412 | 125 |
| Avg. | 25.7 | 537 | 138 |

TABLE 24
Comined Locations CH4 Emission Reductions

| | | |
|---|---|---|
| AVG | KG/HA | 76.5 |
| AVG | % | 15.6 |

Methane measurements showing patterns across dates at the Mercedes locations are provided in Table 25 below. Different letters represent statistically significant differences between treatments at late tillering and flowering stages. The decrease in methane at pre-flowering correlates with the lowest temperature of the season.

TABLE 25

| | Methane Emission Rate (kg/ha/day) | | | | | |
|---|---|---|---|---|---|---|
| | Early | Mid | Late | Pre | | |
| | tillering | tillering | tillering | flowering | Flowering | Maturity |
| Treatment | (4 DAF*) | (18 DAF) | (39 DAF) | (60 DAF) | (69 DAF) | (82 DAF) |
| Control (UTC) | 1.35 | 5.50 | 12.17 B | 5.82 | 7.54 B | 0.14 |
| Rizoderma | 0.64 | 4.45 | 9.49 AB | 5.79 | 7.51 B | 0.22 |
| NLS0737 | 0.71 | 4.23 | 8.19 A | 5.01 | 5.22 A | 0.23 |
| NLS0770 | 0.32 | 4.13 | 8.28 A | 5.58 | 7.13 B | 0.12 |

*DAF—days after flooding initiation

### Rice Yield Results

Rice yield at the Mercedes site was increased over the untreated control 17% by NLS0737 (+27 bu/acre) and 6% by NLS0770 (+9 bu/acre). Due to the delayed planting, the lower solar incidence and the late season rains, the yield from the Ita Ibate site was reduced 44% below the Mercedes site. No differences in yield by treatment were seen in Ita Ibate. At an alpha of 0.15 only the seed treated and sprayed yield (ST/F) from the NLS0737 blocks were considered significantly different from the untreated control. The NLS0737 and the NLS0770 seed treated (ST) yields were similar but not significantly different from each other. The NLS0770 seed treated and sprayed blocks (ST/F) were lower, but not significantly different from the untreated control. The biological control blocks were similar to the NLS0737 seed treated and sprayed (ST/F) treatments.

TABLE 26

| Mercedes | Yield (α) | | % Yield (α) | Bu/Ac | lb/Ac |
|---|---|---|---|---|---|
| Control (UTC) | 158 | a | | | |
| NLS0737ST/F (*) | 185 | b | 17.1 | 27 | 1215 |
| BiolControl | 183 | b | 15.8 | 25 | 1125 |
| NLS0737ST | 173 | ab | 9.5 | 15 | 675 |
| NLS0770ST | 167 | ab | 5.7 | 9 | 405 |
| NLS0770ST/F (*) | 154 | a | −2.5 | −4 | |

(*) Seed treatment (ST) followed by three foliar (F) applications
(α) Different letters represent statistically significant differences between treatments at a = 0.15

### Example 9 Identification of Methylobacterium Strains, Variants and Derivatives

Genomic sequences that can be used to identity and distinguish NLS0737 and NLS0770 from other Methylobacterium strains are identified by an exact k-mer analysis of whole genome sequences of over 5000 public and proprietary Methylobacterium isolates. NLS0737 and NLS0770 are closely related and may be, or originate from, a single Methylobacterium isolate. A 300 nt DNA fragment common to both isolates, but not found in other Methylobacterium strains analyzed is provided as SEQ ID NO:31. Genomic sequences that can be used to identity and distinguish NLS5278, NLS5334, NLS5480, and NLS5549 from other Methylobacterium isolates are identified in the same manner. NLS5278, NLS5334, NLS5480, and NLS5549 are closely related and may be, or originate from, a single Methylobacterium isolate. A 300 nt DNA fragment common to NLS5278, NLS5334, and NLS5480, but not found in other Methylobacterium strains analyzed is provided as SEQ ID NO:32.

SEQ ID NO: 31
AAGGCCCCTCAATATAAGGAGAGATTCCCTACCGGAGTGACGTTTCACT
GCGCAAGTTATACAGATTGCAAAAATCACATTGGTAGCACCTACTCCCC
AAGCTACGCCAATCTCTCCAACTAACGGCCCTACCAGAACAGCAGCTAC
TCGGCCAACGCTTAGTACTGCAGTAGTAAGGAATATCGCTCTGCCATTT
CCATTTGCGAGCAATACGGCACCACATAAGCTGAAAATAGCGTTAACTG
CATAAAACGGTATTAATACTTGCACTAACGCCGTTGCTGAGACCCATTT
TTCCCC
SEQ ID NO: 32
CGCGCCAAGCGATCCTCCGGCGAGACATCCAGCGCGCGATCCTCCGACA
CGTCGAAGGGATGCCCGTTGATCGAGGATCGGGCCTTGTGGCGGATGGC
TGGGTAATCCGCCTTGATCTTCGCTAGGTCCTCCGCGCTCATAGCGCCA
TTGCGCGCCGGTACACTGTAATTGGCCGTCCGCTGGAACACCGTGAGAT
GGGCGGCCTTCGCCGCGATCACCGGAGCGGCTTGGATCCCGGTGGAACC
AGTCCCGATCAGGCCAACCCGCCGGCCCGTGAAATCGACATCCTCATGG
GGCCATC

Assays for detection or identification of specific Methylobacterium strains and closely related derivatives are developed using the disclosed unique genomic DNA essentially as described in WO2022076588 Example 3.

Unique genomic DNA sequences of additional Methylobacterium strains disclosed herein were identified by BLAST analysis of approximately 300 bp genomic DNA fragments using a sliding window of from 1-25 nucleotides and compared to whole genome sequences of over 1000 public and proprietary Methylobacterium isolates. Genomic DNA fragments were identified that have weak BLAST alignments, indicative of approximately 60-95% identity over the entire fragment, to corresponding fragments of a Methylobacterium of interest. Unique fragments from the various disclosed strains useful for assay development are provided as SEQ ID NOS: 33-75 as shown in the table below.

TABLE 27

| STRAIN | SEQ REFERENCE | SEQ ID |
|---|---|---|
| LGP2001 | ref3_25009 | SEQ ID NO: 33 |
| LGP2001 | ref3_25219 | SEQ ID NO: 34 |
| LGP2001 | ref1_4361220 | SEQ ID NO: 35 |
| LGP2001 | ref1_4602420 | SEQ ID NO: 36 |
| LGP2002 | ref4_930 | SEQ ID NO: 37 |
| LGP2002 | ref1_142021 | SEQ ID NO: 38 |
| LGP2002 | ref4_930 | SEQ ID NO: 39 |
| LGP2003 | ref1_86157 | SEQ ID NO: 40 |
| LGP2003 | ref1_142469 | SEQ ID NO: 41 |
| LGP2003 | ref1_142321 | SEQ ID NO: 42 |
| LGP2004 | ref1_194299 | SEQ ID NO: 43 |
| LGP2004 | ref1_194305 | SEQ ID NO: 44 |
| LGP2004 | ref1_194310 | SEQ ID NO: 45 |
| LGP2009 | ref1_153668 | SEQ ID NO: 46 |
| LGP2009 | ref1_3842117 | SEQ ID NO: 47 |
| LGP2009 | ref1_3842278 | SEQ ID NO: 48 |
| LGP2015 | ref1_135566 | SEQ ID NO: 49 |
| LGP2015 | ref1_135772 | SEQ ID NO: 50 |
| LGP2015 | ref1_169470 | SEQ ID NO: 51 |
| LGP2017 | ref1_1185955 | SEQ ID NO: 52 |
| LGP2017 | ref1_3282585 | SEQ ID NO: 53 |
| LGP2017 | ref1_4194637 | SEQ ID NO: 54 |
| LGP2018 | ref1_4871392 | SEQ ID NO: 55 |
| LGP2018 | ref1_1266930 | SEQ ID NO: 56 |
| LGP2018 | ref1_17614 | SEQ ID NO: 57 |
| LGP2019 | ref1_458355 | SEQ ID NO: 58 |
| LGP2019 | ref1_459688 | SEQ ID NO: 59 |
| LGP2019 | ref1_3158527 | SEQ ID NO: 60 |
| NLS0497 | ref1_46464 | SEQ ID NO: 61 |
| NLS0497 | ref1_85227 | SEQ ID NO: 62 |
| NLS0497 | ref1_98103 | SEQ ID NO: 63 |
| NLS0693 | ref1_622066 | SEQ ID NO: 64 |
| NLS0693 | ref1_2496 | SEQ ID NO: 65 |
| NLS0693 | ref1_2640477 | SEQ ID NO: 66 |
| NLS1179 | ref1_687571 | SEQ ID NO: 67 |
| NLS1179 | ref1_695522 | SEQ ID NO: 68 |
| NLS1179 | ref1_705877 | SEQ ID NO: 69 |
| LGP2167 | ref1_54084 | SEQ ID NO: 70 |
| LGP2167 | ref1_4816166 | SEQ ID NO: 71 |
| LGP2167 | ref1_2292077 | SEQ ID NO: 72 |
| LGP2020 | ref1_2810264 | SEQ ID NO: 73 |
| LGP2020 | ref1_322980 | SEQ ID NO: 74 |
| LGP2020 | ref1_2785241 | SEQ ID NO: 75 |

SEQ ID NO: 33
GCCCTTCTGTCAGGCGATATTGTATAATGGCGTTGCCCCAATAGAAGCA
GCCATTCGTGCGAGGGCAGCAGCGACGCTAGGTCGAAAGAGCATCCTAA
TCTCGATCAAGATGCGACTGAGATTTCTGATGAAAATATCTAGACACAA
GCAAAGCTGGTGAAATTACAACGATCATGGCGACAATTGCGGCCAATTC
GGCCGGAACTTGAAGGAACATAAAAATGAATATTACAAATATACCGCAA
AGCATGTAGAGTTGCTACACCAAGGGTCGGGACGTCCAAAAAAACTCAC
TGAGGA
SEQ ID NO: 34
GGAACATAAAAATGAATATTACAAATATACCGCAAAGCATGTAGAGTTG
CTACACCAAGGGTCGGGACGTCCAAAAAAACTCACTGAGGAAGTCGACT
GGAAGCACGAGGCGCCCCCCCCAGGAGCGGGGCGACCGGCAAGGGGGCC
CGCAATTGTCGCCATGATCGACCAGCTTAGGTAGGATCCTCTTTCGACC
TAACGAATGGCTGCTTCTATTGGGGCAACGCCATTATACAATATCGCCT
GACCATCTGGAACGCGGCCCGGTCCACCGGCAGGTTGGCGACGACAGCG
TCGGAG
SEQ ID NO: 35
CGGCGTCGACCAGCCGGGCGAACTGCTTGGGCATGCTCTCCCGCGACGC
CGGCCACAGCCGCGTCCCCGTCCCTCCGCACAGGATCATCGGGTGGATT
TGAAAGGCAAAACGGGACATCAGGATAGGCCGCTCAGGCGTTGGCGCTG
AGGCGCTTGATGTCGGCGTCGACCATCTCGGTGATCAGCGCCTCGAGGC
TGGTCTCGGCCTCCCAGCCGAAGGTCGCCTTGGCCTTGGCGGGGTTGCC
CAGCAGCACCTCGACCTCTGCCGGCCGGAACAGCGCCGGGTCGACGATC
AGGTGG
SEQ ID NO: 36
CTGGACATGCGCCCACCCCGGCCAAGTCCGACCGCACCGGCAACCGCTC
CTGTAGTCGTCGTCATCGTTCTCACCCCTGAGGCGGAGACCGTCCGCTA
ACGGGGTGTCTCAAGCAACCGTGGGGCGGAGGAACACGCACGTAGTCGC
GTTTCAAGGTTCGCACGAACGCCTCGGCCATGCCGTTGCTCTGCGGGCT
CTCCAGCGGCGTCGTTTTTGGCACCAAACCAAGGTCGCGGGCGAAGCGG
CGCGTGTCGCGGGGACTGTCAGGAATTTCGTGTGGGGGCGGCCATAGTG
GATCCG
SEQ ID NO: 37
GCAAAACGACCTAATAGTTCTACAGCGGCATGCGCCAAGTCAGCGCGGT
GAACAGTATACCTGGGAGCAACTTGTCCTCCGAAACCCACATAAAACAA
ATTACTCCTGGCAGTGCCCAGTCCATCAAAATCGAATACAATATTTCTC
GAGGAGGCATCTGTAATAGCCTGCCAAAGCAACAAAGCTATGGCGCCGT
TATGACTTTCATTGCTTCTGGTAGACATAAAATAATATGCCGATTTGTG
ATCCCAAATGTAGAATATTGCCGCATCAATTGCGCCAAGTTTATTTCGG
ATCGAT
SEQ ID NO: 38
GGCGCCAACGGTATGATCGCATGATTTTCCTGCGGCATAGCTTGCGGGA
ATGGCGTATTTGGCGCTCTCCTCAGGAATTTCTAAGGGCATACGCAGGA
ACTCTACAGCACTTTTACTGGTATTTTGTAGTGACAGCGGAGGAGGCTG
GTGCTCAAGGTAATCGTGATGAAGTGATCCGGGCCATTCGGGGCGCGTT
TCTAGTCTTTCCAATCCGCGCCCTGTACCACGTATTACGCCGGACCGGT
CTGCGCCGCGCCGCCCTCTTGACCGCCCTAAATGTCTAAGAGCGTCTAA
CAAAGC
SEQ ID NO: 39
GACGATATCGCTCATCTTCACTGCATTGAAGCTGGTGCCGTACTGCATA
GGGATGAAAAAGTGATGCGGATAGACGGCTGACGGGAAAGCGCCTGGTC
GATCGAAGACTTTGCTGACGAGGTTGTGGTAGCCCCGGATATAGGCATC
GAAGGCCGGGACGTTGATCCCATCCTTTGCCTTATCTTGACTGGCGTCG
TCGCGTGCCGTCAGAACGGGCACGTCGCAGGTCATCGAGGCCAGCACCT
TGCGGAACACCTGCGTTCCGCCGTTGGGATTATCGACGGCGAACGCGGT
GGCCGC
SEQ ID NO: 40
AGCCCACAAGCCTGATGCACTTAACTACATCCTCTAATGTCGCGCCAAT
TTGCTTGGCGGCAGGGGATGTTGTATCGTCATAGGCTTGTCTAACCGGA
ACTTGTTTGCCAATCTCTTTGGCGATCGCAACCGCCATCTCGTGTTCGT
CAACCATGTGCGCGTTCCTCTAATTGCACTCATGGTGCCACGTGCACCT
CCGATCGTCTCGTGTCTAGAATGAAGGTGGGAACAACCTTACACAGGCT
TTCGCGACGCGCGAATTTCTGGTTTCTCCGCCTCGGATGTGGGTTTGAG
CGCTTC
SEQ ID NO: 41
CTTTTCATTTGTCATGATCTCGACCAAGGTATTCACGGCAAGCTCGGTC
TGTTGCTTAGCAAGTGCCTGAACTTCGCGAACGATCGGCTCTCGACCCT
TCGGGTTCGAGACCTGTCCCTTTTGAAAACCACGTGCCCTACACTTTTC
GGGATCAAGGTGCGGGTTGGCTTTGGTCAAAATTCTCTGGCGTCCCATT
ACACGCCCTCCGCATCATCGTTCCCGCGAACGATCTGACCCCCGACTTC
CGCGAGGAAGCGTGTGGCGTGATCCTCGAAGCGGAATGCCACCTCGAAC
TGTTCC
SEQ ID NO: 42
CAGCAGCAAGCAGATCGTTGAAAACCGCTTGAACCGCATCTTGATCGGG
ACCGGAACCAATCAGGTCATCTAGGTAAACCGAGACGTAAACTCGTTTG
CGCTCGGCATCTTTCAGAACGTCCGTGATGCCAGACCGCATTAGTACCA
TCGTCGCCAAGGCGGGCGACTGAACGAAGCCGATCGGCAGAGAGTAACG
GGGACCGCCCCTAATCGGGTTGCGAACGCAAGACCACTTAGCAAAGGTT
CGAGCACGGCCGAACTTCGCATGGTGGAGAGCCGCGGCAACACGGTTCC
GTGATA
SEQ ID NO: 43
GGAAATCGGCTTCAAGTACGACGTCACGCCGGCCATGCAGGTCACGGGT
GCACTGTTCAATCTCGAGCGCGACAACCAGCCGTTCCCCTCGAACGTGG
AGTCCGGCCTCGTCCTTGGCGCAGGTCAGACACGCACCCAGGGCGCGGA
AATCGGCCTGGCCGGCTATCTAACCGATTGGTGGCAGGTCTTTGGCGGC
TACGCTTATACCGAGGCACGCGTACTCTCGCCACTGGAAGACGATGGAG
ACGTGATCGCAGCAGGTAATCTCGTCGGCAACGTTCCGCTAAATACTTT
CAGTCT
SEQ ID NO: 44
CGGCCTGGCCGGCTATCTAACCGATTGGTGGCAGGTCTTTGGCGGCTAC
GCTTATACCGAGGCACGCGTACTCTCGCCACTGGAAGACGATGGAGACG
TGATCGCAGCAGGTAATCTCGTCGGCAACGTTCCGCTAAATACTTTCAG
TCTGTTCAACAAGTTCGATATCAACGAGAATTTCTCCGTTGCTCTGGGC
TATTACTATCAGGATGCCAGCTTTGCCTCCTCAGACAATGCAGTGCGTT
TGCCAAGTTATTCGCGGTTCGATGGCGGGTTGTTCTATCGATTCGACGA
GTTGAC
SEQ ID NO: 45
ACGTTCCGCTAAATACTTTCAGTCTGTTCAACAAGTTCGATATCAACGA
GAATTTCTCCGTTGCTCTGGGCTATTACTATCAGGATGCCAGCTTTGCC
TCCTCAGACAATGCAGTGCGTTTGCCAAGTTATTCGCGGTTCGATGGCG
GGTTGTTCTATCGATTCGACGAGTTGACACGCGTTCAGCTTAGCGTCGA
GAACATTTTCGACAGGCGTTACATCATCAACTCCAACAACAACAACAAC
CTCACGCCTGGCGCGCCGAGAACAGTCCGCGTGCAATTGATCGCTCGGT
TCTAAA
SEQ ID NO: 46
TAGACATTCCAACAAACCGGCAAGAGGCTCGTCCTCACTCGAGGATTTG
TTGGGACTTGCATGATGTCGAAGCGGAGCCGTTATGACCTGGGTGCGAT
CATGCGCCGAGCATGGGAGATGGCTCGGGAGGCGGCATTCGCGGTTGGC
GAGCGGGCACGGACTCACCTTGCTGCCGCGATGCGCAGCGCGTGGGCCG
AAGCCAAGTTGGCACTCGCGCCCACGAAGACGGAGCAGGATCGTCTCTC
TCCGAGCGACATGATCGGACATGAGGACGCCTACCAAGGCCGGGTTCTA
AAATAT
SEQ ID NO: 47
AAGATGGATACGACAAGCGCGATTACATTATTTGCGAAATAGATGGACA
AATAAAAGACAAAGGACTGATGTATTTCCTTAAATCTGGACAAGTTGAC
CTCTTTCACATAGAAGTCACCACTCCCTTTGGGACAATTTGGTGTCACG
AAAACATAGAGGCCGAACTTCTTAGCTGAATTATCGCGCTCCGGGTTCT
TATGCGGCTGAGTGAAGCGCGGGACAGCTTGCGAGCAGGGCCGCCAATG
GCAGCCGGGATGACACAATGCTCGGTCTCCCGACGCTTCTTCAATCGGG
AGCGCT
SEQ ID NO: 48
AGCTGAATTATCGCGCTCCGGGTTCTTATGCGGCTGAGTGAAGCGCGGG
ACAGCTTGCGAGCAGGGCCGCCAATGGCAGCCGGGATGACACAATGCTC
GGTCTCCCGACGCTTCTTCAATCGGGAGCGCTTCGCAGCCCGGGGCGGC
GCGCTCATGCGTCACGACCTGGGCCCTGCGCACCTTCGCGGCCCCGCCG
TCCCGGCAGATCCCTGATGCCCCAAGTGGGCGGCCACTCCATCAAAGAA
CCCCGGCCTGTGGCAGATCTCGTAGGCATACCGAGGTTCCGCAGTGCCC
CCACC
SEQ ID NO: 49
ACGGTCACCCCACGGACTGGGCGAGTACCTCACCGGTGTTCTATCATAA
CGCCGAGTTAGTTTTCGACCGTCCCTTATGCGATGTACCACCGGTGTCG
GCAGCCGATTTCGTCCCACCGGGAGCTGGCGTTCCGGTTCAGACCACCA
TCATCGGTCACGATGTCTGGATTGGACACGGGGCCTTCATCTCCCCCGG
CGTGACTATAGGAAACGGCGCGATCGTCGGGGCCCAGGCGGTCGTCACA
AGAGATGTCCCACCCTATGCGGTAGTTGCTGGCGTCCCCGCGACCGTAC
GACGAT
SEQ ID NO: 50
CCAATAAAAGCGTTGGCCGCCTGGGCAACCCGATCCGAGCCTAAGACTC
AAAGCGCAAGCGAACACTTGGTAGAGACAGCCCGCCGACTACGGCGTTC
CAGCACTCTCCGGCTTTGATCGGATAGGCATTGGTCAAGGTGCCGGTGG
TGATGACCTCGCCCGCCGCAAGCGGCGAATTACTCGGATCAGCGGCCAG
CACCTCGACCAAGTGTCGGAGCGCGACCAAAGGGCCACGTTCGAGGACG
TTTGAGGCGCGACCAGTCTCGATAGTCTCATCGTCGCGGCGAAGCTGCA
CCTCGA
SEQ ID NO: 51
CGATGGCACCGACCTGCCATGCCTCTGCCGTCCGCGCCAGAATGGTAAA
GAGGACGAAGGGGGTAAGGATCGTCGCTGCAGTGTTGAGCAGCGACCAG
AGAAGGGGGCCGAACATCGGCATCAAACCTCGATTGCCACTCGGACGCG
AAGCGCGTCTTGAAGGAGGGATGGAAGCGAAACGGCCGCAGAGTAACCG
CCGACGAAAGATTGCACCCCTCATCGAGCAGGATCGGAGGTGAAGGCAA
GCGTGGGTTATTGGTAAGTGCAAAAAATATAATGGTAGCGTCAGATCTA
GCGTTC
SEQ ID NO: 52
AGTCATTGATCAAGCAACCCCTATTGAGTTGGATATCGAAGGATCAAGG
TCGCGTCAATAGATGCATCTATCAGGCCAAATGTCGCTTTTCAAGAATG
GCTCTTTCGAAGCTATCTTTATAATCGCTCGCCATTCTCTCATTACCAA
AATCGACCTTAACTAGCTCGACATTGATGCGAGCAGCTCCGGCAAACGA
GGAGAGATTGACCTTAAAGGAATTGAACGCCTCAAGCAATTCAGACACA
TTACCAGGAGTGCTATAGCAACAACCAGACCCATATCGGTCAATAACCT
CTTTTA
SEQ ID NO: 53
CGCAAAACGATTTATCACTGCCATCTTGTTGTTTGATAACCCTTTTTTA
CCAGACGTTATGCTGGGCGAGAAAGAGGACTAGCAGATCGGAGCGGTAT
CGCGATTTTTCGGTAGTTCGCGCCTACAACAGGATAAGATCCGATAGTG
AAGCAACATGGCTGTTTTTTGATTTGTAAGTCAGCAACTTAAGCAGCCA
GCCTATCTGCCGTCGCAGACGCTTGAGGCATCGGGCAGCATCTTAGAAA
AGGTGGCAGTAATTGCCACAGCGGAACGTAGCGGCACGGATAAGCACGC
AGGGTC
SEQ ID NO: 54
CCCATCTGGACCCAATATCCCCTTCATCGACAATTCCCGAGTAAGTGTG
GGTTCGAGGATTTCGCGAAACAGCCTTGTTCGTTCCTCCGGCCTTAAAA
TTGGCGTGCCGTCGGGAGATCGATAGGCATCCCTTACCTGCCTTTCGAC
CGCCGGCACACGCGCGCCGGTCGTCGTGTTCACGGCCACGGAATGGACG
AAGGTGCGCCGCTCATTTCGCTCGTTTGCCGTCTCCACCATCCAGGAGG
CCAGCAGGACGGTTTCGTCTCGACCGCCGGTCACACACACCGCAAGGGA
CTCAGG
SEQ ID NO: 55
ACCTGCTAAAATCACGTCCTCTCAGATTGAAAAATCATTGAAGAAACGT
GTCGAACGATTGCCGGGGATTATGACGTTAGATCAATTGAAAAATACAA
GCTTTGAAATTGAGTTACAGCCAAAAGATGCCCCGGATCCGGACCCATC
AGACTTCGGTGGCTAGTTCGAGCCAAACTCGAACGTCGCCATGGCGCGC
AAGTCGCAATACCATTTCACAGCGCAGCGGTTATTTCGTTGTACACTGT
AGCAATGCGTCGGCTTGCGCGCTTCCGCTGGCGATCAAAGGTCCGCCGA
TTTACG
SEQ ID NO: 56
TCCCGAACATACAATGGAGGAAGCGTGTGGTAGGCCAATTTGTAACGAA
ATATGGCATCGGTCACGGCTCTCTCAATAAATTCGATCTCAAGTCTTCT
GAACGAGCATGCCTCATCCTTATCCTGAGCGAACGCCTGCCAGTTTGCA
GTCATTCCAACATACATAGCCAAAAAGGCGAGGTAGACCTTCATACGGG
CACCTCAATCGTCCCCATTCGTTCAAGCTCCTTCAAGATAACAGCCGCA
CCACATTGCTGAGATCGAAGATTCGGATCAAATATTCCATCAAATTTAT
ACTTTC
SEQ ID NO: 57
GCATCCTTTGCGCTCGCAGGCCTAAGGTCAAGCCCGGTTACTTCGTTTG
GTAGAACGAGGTAGACGATGCCTAGTCTTAAGGTGGCCCATGTTAACCA
ACAGGGCCAGAACATGATTATAGTTCCGTTAGATGCCAACTTCGGTTAC
AAAACCGATGGTGAGCAGTCCGACATCATGTTCGAAATACAGGACGCGG
CGCGGTCCGCCGGTCTTGCGGGTGCCGTAGTAGCGTTCTGGCAGTCAGG
TGGACAAACCCGTTTCCGGGGCCCGGCTCCGTGGCACCCATTCCTTCGC
AGCCTC
SEQ ID NO: 58
CAACTATGTAGACCCGACGGTGCGATTTCACTTCGCAAAGCCGCAGGGC
AGCACCCTTGCGCTCAATGTTGACGCCAGCGTGATCTATACTATTACCG
TCACGCACACGCAGGGCGGCGTACAGATTCATCGCGAGAGTAAGAACCA
CCATCAGACCATCACGCGCAGCGACCTGAGCAAGCAGTTCGGCGTTGGT
GTGGCCGACCAGCTGACGCGCGATCAGGTCATGAAGGTGATCGAGTCGG
CATTTCGCGACGCTACCCGCTAAGATCGGCGCCCACGAAACGCTACGAG
ACTAGG
SEQ ID NO: 59
AGCCGGCATCTTGTTCAAGGCGCTCACCTCGACGCCGACGCTGTAGGCG
ACTTGAGAGGGCGTCTCATATGAACGAAGCATCTTCGCGTAGAGAACCT
TCTTGTTCTCCTGCGTGATGTTCGCTTTGCAGACGTTGACTGCCGCCAT
GAACGCCGAAGCCTTGCGCGCTTCATCGTAATCGCCTGCGAAGGCGGGT
AGTGAAAAGCTTAGTGCAATGGCAAACACAGCCGCCGAACGTCGCATGG
TATCCGTCCCCGATTGACGGCAGTGCCGCCATATCTCGGCTTTAGCAGA
GCTGAT
SEQ ID NO: 60
AACCTGCGCCGGCCGAGGTTTCGCGAGCCGTCGCCACGGGCAACGCCTC
GCCCGCGATGTGCAAAAAAGTCCCCGGCACTTCGCGCCGTCGTCCGATC
CACGACCGCGAATTTCTCAACGAGTACAAGGTGCTTATGGGAGATCCGA
GCGTCCGTCCCGGAGCCCGAGACCGCGCGGCCCGAGTAATAGGCGAAAA
AGACTCCTACTCCTCGGGCTTCTCGGGCCCCCTCAGCAACATCTACGCT
TGCCGCCCATCACCCTGGCGGGAGATCAGCGACGAGACACAGGCCCACT
TCGCCC
SEQ ID NO: 61
CTCAACGTTGCCGCCTTAACCGAAGACTAACTAACATAAATTTCAGTTA
GCCGCGAAACAGAATATGATACTTGGTCTTATACGACGCATAGAGGCGC
ATTCCGTCAAGTCGCGCATTGCAGAGTCCCCCCGAAAATTCGTTTTCTG
GGAGCGAGCAGAAACGTGTGCACGTGTTCTGTCACTCTATTTTCTGGTT
TAGAGCAATCATCTCGCGGCTCAGATCCCGTCGTTCCATAGAGTGGTTT
CCGGTTCAGTATGAATTGTGAGCAGCCCCTGCGGCTATATCTACTTCGA
CGTATA
SEQ ID NO: 62
ACCAACAGGCAGAAGAGGCTTACAAAAATGCTCTAGATATCTATAATCG
CGCCGGTCTGGATAATAGGCGGGAAAAGTCAAATATTCTTATCGGACTT
GGCGATGCAGCTAGCGCACTGAGCAAATTTATAGACGCGAAAAATTTCT
ACAGTGAAGGTTTAGCAGTCCGAGCGCGGTCATGACGGTGCTTGCGGAC
TAGTGTCTGCTCGCAATTCAGAGCAGCCGCTTCGGCTGGTTCAGTTTGG
GTATGCCCTTTCTGTACCAGCCTGTGACTGGTCTGGTATGTAGCCCTTG
ACCAAT
SEQ ID NO: 63
ATGAAGCTCCGTCGTGTTTATGTAAAGAACGTTCGCAGTTTCTACGATG
CTGAAGAACTCATTCTGGACGGAGATATTTCTATTATTATAGGCCCAAA
TGGTGGAGGAAAAACAAATCTACTTGATGCGACGATACACTTGTTGAGA
AGACATTTATTGCAATCGTGGGCTGTCGTTCGCCATACATACACGCCAA
TAAACTATTCCGATCACTTCCAACCAAATGATCAAATCACAAACTATGC
GCTGGAGAAGTACAGCGGTCGCGAAAGCGAAGACCAAGTCGTTGAGTTC
GATATC
SEQ ID NO: 64
CGGGGGCGGTCTCCTCATATATGGAGGGAATTAATCTGAAGGGTCGGAG
CCCCCGCTACGGCACACACACGGCGGCGGGCGGTGGGCTCCTTCCTCAT
ATACGCTTCCTGATAATTTCGACCGTAATCGCCCGCCGGCCGAGCGGCC
AGACCTTATTAGTCCAGCATCACACGGTGCATCGTATCGTGGAGGGCGA
ACAACCGCCTAAGGTTCGCCACGTAGCCATCCACATCCGCCCTTGCCGC
TCGCGCAAGGGAAGCAGAATGCTTGATAGCGACTTCGCGGGCGTCTTTG
GTAGCC
SEQ ID NO: 65
TGTGGGCGCTCATGGAAACGGGGGACGTGCTTTTTTGATCTCTAGCCAT
TGTCATCATCCATCGGTCCGGGTGACAGGCCTTTACGCACATAACAACG
GTTATTTCGATCAGCCCCCTCCTGATCGCACAAGTCAGCTATACAGATG
CTTCTGAAAGCCAACAAAGGCGTTACGTATCTGCGTGACCCCACCGAGC
TGCGCTGCGGCATCGCTGACAGCTCTGTATTTTAATTCGAGGAGGTGGG
GCAATTTAGCCTGGTCAAGCTCGCCAACGTCTTCCTTCACATATTGAGA
CAGGAC
SEQ ID NO: 66
AGAGTGTATTTCCCCAAGACTTTATCTCTTTGCTCATCACAAAACAGCC
CGCTCAAGATGATATGCGCTGGGATTTGTGCCGCGACCCGTTGTAATTG
TATTTACCTCTCGGCTCATGTTCCCTGTCGCCGGAGCGATTGGAGGTAA
AGTAATCTCAACTTTGCGGCCCATGGCTGATGCTAGCTCAGCTACGCGA
CCTAAGGTAATATCTTTGTGTCCGCGGAGTTCGCGATGAATGACCGAGC
GATTGACGCCGATGGTGCGGGCAATATCGGCTTGAGTTAGACCGTTCTT
GGCTTC
SEQ ID NO: 67
ACCTGTCTCGACATCGGGGGCAGCCACGAGGCGTCGGCCGTGGTAGATC
TCGGCGAAGCGGTAGTAGTGGGCGAGGTCCCCATCGGGATCGATGGGGG
AGTCCGGCGTACCTTCTCCCTCTTCTGCGATTAGCGACAGGGCTCGCAT
GGCGCTGGGTGCGTCAGTGATGGCGAACAACCTACTATCGGGGAACCAC
CGCCTGTCGACGAGCTGGCGGTCCGGGTTTCCTTTGAAGACAGCGTCAC
CCAGCGCTTCGATCTTCTCAATCAAGGCTGCGTAGAACTGCCCGATGGT
CGCGAA
SEQ ID NO: 68
GACGTGGTGACGGTGGCACCAGTCGCCCAGCGAAGCGGCTACTGTACTG
AGCCCGCAGTCATGTCGGCTGAAGAAATGGTCGATATGCCCCAGGGAGG
TCGGGCGTAGGTCGTACCTCAAATTCACCGGCAGGATCTTGTCCAGCAA
GGCGTCCGAATTCGCATCGACCTGACTTAGGAAATTACCGAGGTAATCG
CTCGGTCCGTTCGCGTCGGCGGCCTGCATCGCCAGCAGCGCCTTATGCT
CGGTCGTCAACTCACGAGCGAATATTGTCCAGGACAGGTTGTCGACCTC
GGCGAC
SEQ ID NO: 69
GAGCATGGCAGGCCACCACGCATCGCTTCTTCATCACCTTGCTCGGACG
ACTTGCTGCTTTGTCGGTCTCTCTCTCAAGGACGAGACGCTGCGACACT
TGCTTCGTCAGAGTGCCAGGATTAATCCCGGTCAATACCACTACTACGT
CAGCTGGCGTGCGCCGGGCGGCACGAGGGACGTCGACGCGGAACGATTG
ATTCGAGACGCTAACTTTGACGTGTACAATTTAGTCACGCTTTTCCTTA
GCGACGCCGAGATAGCCTCGTTGGGCCGCTTGCTCACCGCCGATGCAGC
CGAGTT
SEQ ID NO: 70
AACGCGATGGGAGCAGCACGTAATGGGCGTCATACAAGATATAAAAGAA
GGACTTGAAGGTAGAATAGATAACCTTAAGGAAAATATTGATAATATCA
GTTCCAGTATCACATGCACAATACAAGGAGTGCCGAAGGTCGAGTCAGA
AAGAATTTTGGGATGTGCAAACGCTTCGTTTGACCAAATACAGAAAATG
TGGATGAATGGTCCTACGGATAGGGCTGCATTTCTCGACGCCGTGAAAA
GTTTGATAGACCGCAACGCAAGTAATGGAAAATTCGGAGTTCCAGGATC
ATATTG
SEQ ID NO: 71
TTTTCCTTTCGCGCCTATGACCTGAGTGCGGCCGAAGCTGTCCGCTTAT
TAGAGTTTTACAACCAGATGCGAGCGGCTTAGCTCTTCCTCCGTTATTA
AGCAGGGCGCCTCTTCTTAGGGGCGCCCCTTCATATTTAATCTTGTCTG
ATGTCTGGCGCCATATCAGACAAACATCAGCGTTGACCTTGATTTTGCA
TCTACATTAGGGTTGCTCCCAGAATGGAGCCGCCGATGTCCGTCCCGTA
CCTCAAGCGAGAGATGTGGGGTGTGTATTACATCCATTGGCGCGAGGGC
GGCCGC
SEQ ID NO: 72
GTTGGAGATGGTGCCGTTTGCAACCGTCAGATCGCCTACTGTGAATCCG
GTGACCTCCCGAGAGAACGTGAAGGTAACTGTCGTAGTCTCGCCAACTG
CCAAAGAGGTGTCCGCAACGGCGATGGTCGCTGTCGGGGCGCTTGTTTG
GACATCAACGGTGAGACCCGCCGAGGCAGGCCCCGTGTTACCAGCGGTA
TCGACGGCCCTAGCGGTGACCGTGTGCGATCCAGCCGTTAGCGTCGAAC
TGGTGATGCTATAGGTCCCGCCGATCGCCACTGCCGAGCCCAGAATGGT
CGTGCC
SEQ ID NO: 73
ACCGAAGGCGTCCCCGGACACGAAGGCCTGAAACACCATATCTGTGGCG
ATCAGGCCGACGTGGTCGCGGACTTCAACTGGCAGAGAATGCCAGGCCG
CTTCGATTTCAGATGATACTGGTACGGACATAGGAGCGGCTTAGCTTTC
TCAGTGCAAATGTGATTGATTCCGGCTCAAAAATGATCTTGATCGGACG
AGACGTTTTCAATCCATGTCGTGTTGCCATCGCCGATCGGTGCGTCAAG
AGACAGATGGCGCCGACCGTAGATACGCGTTCGGGTTGCCCGCACCGCT
TCTCCA
SEQ ID NO: 74
GGAGGTGTGATCTGATGATGTGCTGGATGAAATTGGCGGTCGAGCACTT
GTTCAGCTTGGCCAGCTCGACGAGATCGGCGTGATGCTCGGCGTCGATC
AGGATGTTCAGCGAGACCGGACGTACGCAGGACTTGGTATTAGCGCCGT
TGCGCATCAGCTTGCAGCCTTGCTCTGCTTCTCAGCGTGCCGCGTCAGG
ATGACCCTGATGTAGCTGTTGAGGTTGATGCCGTAATAGCCTGCGGACT
CTGTGAGATCCCGGCGAAGATCGTCGGCGAGGGTCAGGCGGATGGTGCT
GGTCGG
SEQ ID NO: 75
AAGTAACCGCTCAACATGATCTTCAGCATGTTGTCCAACAGCAGGAGAA
TACATGTAATTCACCATGACCGGCAAGCTGCGACTGGCCATTGCTTCCA
CCGCTTGAATGTAGCGATCGAATTTCGCAAAATCAGGGTGGAATGAAAA
TATCGAACCAAACTGCGAGCCTTGAATCCGTTCTGCAAAATTATCGAAA
AATTTTCTTGGCCGACTGCCGTTCGAAAACATTCTTACGTTTACATGCG
GCCCGCCTGAAACAAGACAGTCTACCAGCTCTGGGAAATGGGGGTGAAG
GGTCGG

### REFERENCES

Green, P. N. 2005. Methylobacterium. In Brenner, D. J., N. R. Krieg, and J. T. Staley (eds.). “Bergey's Manual of Systematic Bacteriology. Volume two, The Proteobacteria. Part C, The alpha-, beta-, delta-, and epsilonproteobacteria.” Second edition. Springer, New York. Pages 567-571.
Green, P. N. and Ardley, J. K. 2018. Review of the genus Methylobacterium and closely related organisms: a proposal that some Methylobacterium species be reclassified into a new genus, Methylorubrum gen. nov. Int J Syst Evol Microbiol. 2018 September; 68(9):2727-2748. doi: 10.1099/ijsem.0.002856.
Konstantinidis K. T., Ramette A., Tiedje J. M.: (2006). The bacterial species definition in the genomic era. Philos Trans R Soc Lond B Biol Sci 361: 1929-1940.
Lidstrom, M. E. 2006. Aerobic methylotrophic prokaryotes. In Dworkin, M., S. Falkow, E. Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
Sy, A., Giraud, E., Jourand, P., Garcia, N., Willems, A., De Lajudie, P., Prin, Y., Neyra, M., Gillis, M., Boivin-Masson, C., and Dreyfus, B. 2001. Methylotrophic Methylobacterium Bacteria Nodulate and Fix Nitrogen in Symbiosis with Legumes. Jour. Bacteriol. 183(1):214-220.

The breadth and scope of the present disclosure should not be limited by any of the above-described embodiments, but should be defined only in accordance with the following claims and their equivalents.

1. A method for mitigating methane gas in an agricultural field that comprises:
(a) applying a composition to a field, plant, plant part or seed, wherein the composition comprises at least one Methylobacterium comprising a soluble methane monooxygenase enzyme; and,
(b) growing the Methylobacterium whereby the Methylobacterium uses methane as a carbon source;
wherein the use of the methane as the carbon source oxidizes methane and reduces methane emissions in the field.

2. The method of claim 1, wherein the composition comprises at least one Methylobacterium selected from the group consisting of NLS0737, NLS0770, NLS5278, NLS5334, NLS5480, NLS5549, and variants thereof.

3. The method of claim 1, wherein the plant, plant part, or seed is rice.

4. The method of claim 1, where the composition is applied to a flooded or irrigated rice field.

5. The method of claim 1, wherein application of the composition provides for increased yield of a crop grown in the agricultural field.

6. A method of claim 1, wherein the composition further comprises one or more second biological.

7. The method of claim 1, wherein the composition further comprises one or more obligate methanotroph.

8. The method of claim 1, wherein the composition further comprises a second Methylobacteria wherein the second Methylobacteria enhances growth, yield, nutrient update, or nitrogen use efficiency.

9. The method of claim 8 wherein the second Methylobacteria is selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2022 (NRRL B-68033), LGP2167 (NRRL B-67927), LGP2029 (NRRL B-68065), LGP2030 (NRRL B-68066), LGP2031 (NRRL B-68067), LGP2033 (NRRL B-68068), LGP2034 (NRRL B-68069), variants thereof, or a combination thereof.

10. A method for selecting a Methylobacterium isolate capable of utilizing methanol as a food source, wherein the method comprises:
a) detecting in the genome of a Methylobacterium isolate, a genetic element, wherein the genetic element comprises a component of a soluble methane monooxygenase; and
b) treating a field, water, plant, plant part or seed with the Methylobacterium isolate, and measuring green-house gas emissions.

11. The method of claim 10, wherein the plant is a rice plant.

12. A method for mitigating methane that comprises:
(a) treating pasture, wasteland, a landfill or waste, or land with livestock feed, with a composition comprising at least one Methylobacterium isolate; and
(b) growing the Methylobacterium in the pasture, wasteland, a landfill or waste, or land with livestock feed, thereby mitigating methane.

13-22. (canceled)

23. The method of claim 1, wherein the Methylobacterium is present in the composition at a concentration of from about 1×10³ CFU to about 6×10¹⁰ CFU.

24. A composition comprising a fermentation product comprising a Methylobacterium strain, wherein said fermentation product is essentially free of contaminating microorganisms, and wherein the Methylobacterium strain is selected from the group consisting of NLS0770, NLS0737, NLS5278, NLS5334, NLS5480, NLS5549, and variants thereof.

25. The composition of claim 24, wherein said composition further comprises a Methylobacterium selected from the group consisting of LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), NLS0497 (NRRL B-67925), NLS0693 (NRRL B-67926), NLS1179 (NRRL B-67929), LGP2167 (NRRL B-67927), LGP2020 (NRRL-B-67892), LGP2021 (NRRL-B-68032), LGP2022 (NRRL-B-68033), LGP2023 (NRRL-B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof.

26. The composition of claim 24, wherein said composition further comprises at least one additional component selected from the group consisting of an additional active ingredient, an agriculturally acceptable adjuvant, and an agriculturally acceptable excipient.

27. The composition of claim 24, wherein said Methylobacterium comprises a soluble methane monooxygenase.

28. The composition of claim 27, wherein the Methylobacterium comprises NLS0737 or NLS0770.

29. (canceled)

30. A plant, plant part or seed at least partially coated with the composition of claim 24.

31. The plant of claim 30, wherein said plant is a rice plant.

32. (canceled)

33. The composition of claim 24, which further comprises an ogbligate methanotroph.

34.-38. (canceled)


SEQUENCE LISTING (75 sequences)

SEQ ID NO: 1 - protein, 122 aa, Methylobacterium sp.
MSTSQPTSQNTIFQKMDTMKFERTISHQCGVTMNDSVEARAIAEYMSEKPDVTVTYQPALIRIDGVGKLTFLMDEISEILGREMTAELFEVNASTHYGRMIRIDDNTVALFGSMEEALEYIE

SEQ ID NO: 2 - protein, 356 aa, Methylobacterium sp.
MSEQQTEVVKSGAAGSAVFADSDSRKYRYFEPRGKRATHYEDVTVDVQPDPERYLKQDWIISFANGDGAYTKQSTAVKSSNWHAFRAPDQEWERTHYQRQSKIETMVQSVIANGRRAGAPKAYDKAWVKVLQNHLGAWKHAEFGLGTSMMQAQRYGYTQMINNATLTNSSYKLRLAQDITLYLSEIGMDIQGFDDEAGKIRWLEDPIWQDTRLAIETIMGSSDYLEQYFATNIVFEPLIGELFRSGFLMQAASPNSDFITPSVISAAEADYERNLANTIDLFHLLITDGEFASQNGALFQDWVTKHAALAHKAANTLQPIWSQPHAKPVQFSDARASSLERFSKILGELGLETPKE

SEQ ID NO: 3 - protein, 126 aa, Methylobacterium sp.
MSPLWSILHDFLSSGEDARDIHFFYGARTEKDLFYIDKFAAITAEYPRFKFIPVLSNADEDTEWQGETGFVHQVVGTHLKRLDLGDDLDVYACGPTPMIDALTPVLFMNDVDTDRIYYDRFTPAPA

SEQ ID NO: 4 - protein, 556 aa, Methylobacterium sp.
MSGALTLNKITNQRGMPVGEAAKRIADLGWNPSYVQEAMTFPTDYKITKAPKDPMKQVLRSYFPMQEEKDNRVYGALDAALRGDMFRNVQPRWVEWMKLFLAIIPFPEISAARAMAMVGRLAPGEELRTGFTMQMVDEFRHSTIQMNLKKWYMENYIDPAGFDITEEAFGKCYATTIGRQFGEGFITGDAITSANMYLTVVAETAFTNTLFVAMPSEAARNGDYALPTVFLSVQSDESRHVGNGHSMLMAMLKEPDNHLLLERDLRYAFWQNHAIVDAAVGTFIEYGTTNRDKNKESYAEMWHRWIFEDYYRTYMLPLEKYGIKVHHDDVQAAWDRLTKKFYVHAVAQFFSVGWPVNFWRIEAQTEKDFEWFEHKYPGWYAQFGEYWKWYEKLSHKGQKVLLFNEDVGYVYPHRCWSSLVPCLIREDIVVDEIDGKLHTFAHEIDRWTAVEAFSGEYQGRPTPAMGRFSGRREWEDCYHGWDLADAIKDLGFVRTDGKTLVPQPHLRFDNKEMWTLDDVRGHTLQSPLQMLRAMTPSERDAHLTQYRAGFSINPCN

SEQ ID NO: 5 - protein, 123 aa, Methylobacterium sp.
MSVNARNASNQNIFQKMGDLSFDQTISHQCGVTMNDSVEARAIAEFMGQKPGVTITHQPALIRIDGNGELVFKMDEISEILGKEMTAEIFEVNTSTHYGRMIRIDDNTVTLFGDMEAALQYIE

SEQ ID NO: 6 - protein, 355 aa, Methylobacterium sp.
MTLEAATRKSGAAGSAIFADSDSRKYRYFEPKGKRATHYEDMTVDVQPDPERYLLQNWIISFADGKGAYSKENTAALSSDWHAFRAPDQEWERTHYQRQSKIEAMVQSVIANGRKAGAPAAFDKAWVKILQNHLGAWKHVEFGLGTALMQAQRYGYTQMINNATLTNSSYKLRLAQDITLYLAEIGMDIAGFDDTAGKRHWLEDGTWQGTREAVEAIMGSTDYLEQYFATNIVFEPLVGELFRSGFLMQVASSNGDFITPPVISAAEADYERNLANAIDLFHLLATDAAHAEHNRTLFRSWVDRHVALARKAAADLQPIWSQPHSKPAQFADAKAASTERVTKILGELGLDLPQE

SEQ ID NO: 7 - protein, 345 aa, Methylobacterium sp.
MSEVYTVKLNPVGVEFEVEEDETVLDAAFRQGIALPHGCKEGRCAACKCIVTDGEVEMLPYSTFALNETERDQNHVLLCRSLISSDIEVDLLNYDEELLSKSIPVRDLQGRVSRVSSLTHDIRLLEIELDQPLKFWAGQYVDITLPGAEEITRSFSMANPPSETQKLSFIIKKYPGGRFSSRLDGELDVGTPVGVKGPYGTCFRRENRGGALILVGGGSGMSPLWSILHDHIASGEERPVHFFYGARTQTDLFYLDLFAEVARSNPRFTFIPVLSHASDDAAWQGERGFVHEVVNDHLRRLAYGEDLDVYACGPEPMIEALTPVLQMNDVSSERIFFDKFTPART

SEQ ID NO: 8 - protein, 556 aa, Methylobacterium sp.
MSGTLTLNKITAQKGISVGEAAKKIADLGWNPSYVQEAMTFPTDYKITKAPKDPMKQVLRSYFPMQEEKDNRVYGALDAALRGDMFRNVEPRWVEWMKLFLAIIPFPEISAARSMAMVGRLAPGEELRTGFTMQMVDEFRHSTIQMNLKKWYMENYIDPAGFDITEEAFGKCYATTIGRQFGEGFITGDAITSANIYLTVVAETAFTNTLFVAMPSEAARNGDYALPTVFLSVQSDESRHVGNGHSMLMSILKEPENHLLLERDLRYAFWQNHAIVDAAIGTFIEYGTTNRDKDKESYAEMWHRWIFEDYYRTYMLPLEKYGIKIHHDDVQTAWKRITEKHYVHKIAQFFSVGWPVNFWRIEAQTEKDFEWFEHKYPGWYAQFGEYWKWYGKLSRPGQKIVTFNEDVGYVYPHRCWSSLVPCVVREDLVTDEIDGQLHTFAHEIDRWTAVEAFANEYEGRPTPAMGRFSGRREWESVYHNWDLADAIKDLGFVRTDGKTLVPQPHLRFDNKDMWTLDDVRGYTLKSPLITLREMSPEAREAHLAEYRAGFTINPCN

SEQ ID NO: 9 - DNA, 369 bp, Methylobacterium sp.
  source 1..369: mol_type = genomic DNA
atgtcgacatcacaacccacatctcaaaataccatcttccaaaagatggacaccatgaagttcgagcggacgatctcgcatcagtgcggggtcacaatgaacgatagtgttgaagctcgggcgatcgccgaatacatgtcggagaagccagatgtcactgtgacttatcaacctgcgctcatccgcatcgacggggttgggaagcttacgttcctgatggacgagatcagtgagatcctcggccgagagatgactgcggagcttttcgaggtaaacgcttctacgcactacggtcgcatgatccgcattgatgacaacacagttgcactgtttggcagcatggaagaggcgctcgaatacatcgagtaa

SEQ ID NO: 10 - DNA, 1071 bp, Methylobacterium sp.
  source 1..1071: mol_type = genomic DNA
gtgagcgaacaacagaccgaagtggtgaagtcaggagctgccggctcagccgtattcgcagactctgacagccgcaagtatcgttacttcgaaccgcgcggcaagcgggcgacgcactatgaagatgtgacagtcgatgtccaacctgatcccgagcggtatctaaaacaggattggatcatctcctttgccaatggcgacggagcttatacgaaacagtctaccgccgtgaaaagttccaactggcatgcgttccgggcgccggaccaagagtgggagcggacccactatcagcgtcagtccaagatcgagacgatggtccaatccgttatcgccaacggtcgaagagctggagcccccaaggcttacgacaaggcctgggtgaaggttctgcagaaccatttgggtgcgtggaagcacgccgaattcggcctgggcacctcgatgatgcaggcccagcgctacggctacactcaaatgattaacaacgcgacgttgacgaactcgtcgtacaagcttcgtcttgcacaggacatcacgctttacctctcggaaatcggtatggatatccaggggtttgatgatgaggccggcaagattaggtggctggaggatccgatctggcaggacacgcgcctcgccatcgaaactattatgggttcttccgactatttagagcagtactttgcgacgaacattgtcttcgaaccactaattggagagcttttccgatcaggtttccttatgcaggccgcatccccaaatagtgacttcattaccccgtcggtgatctcggccgcagaggccgattacgagcgcaatctcgccaacaccattgacctattccatctgctcataacggacggtgaattcgcatcccagaatggtgcgctgttccaagactgggtcacgaagcatgcggcgctcgcgcacaaagctgccaacacgctccaacccatctggtcgcaaccccacgcgaaaccggtccagttttccgatgccagggccagctcattggagcggttcagcaagatcctcggcgaacttggcctcgaaacccccaaggagtga

SEQ ID NO: 11 - DNA, 381 bp, Methylobacterium sp.
  source 1..381: mol_type = genomic DNA
atgtcgccgctgtggtcaatcctgcacgacttcctctcgtctggtgaggacgcgcgcgatatccacttcttctatggcgcgcgcacggagaaagacctattttacatcgataagttcgcagcgatcacagccgaataccctcggttcaaattcatacccgtgctctctaacgcggatgaggacaccgagtggcaaggggagacgggcttcgtccatcaagtcgttggaacacacctcaaacgccttgatctcggcgatgatttagacgtctacgcctgcggtccgacaccgatgatcgacgcgctaacaccggtgttgttcatgaacgacgttgacaccgaccggatctactatgaccggtttacccctgcgccggcctga

SEQ ID NO: 12 - DNA, 1671 bp, Methylobacterium sp.
  source 1..1671: mol_type = genomic DNA
atgtcgggagcactaactctcaacaagataacgaaccaacgtggcatgccggtgggcgaggctgcgaagcgtattgcggatcttgggtggaacccgtcgtacgtccaggaagccatgacgtttcccacggactacaagatcacgaaggcgccgaaggacccgatgaagcaggtccttcggtcatacttcccgatgcaagaagagaaggacaaccgcgtatacggcgcactcgacgccgctttgcgcggcgacatgttccgcaacgtgcagccgcgttgggtcgagtggatgaagctgttcctggccatcatcccgttcccagagatctcggctgcgcgcgcgatggccatggtggggcggcttgctccgggcgaagaactgcgtaccggcttcaccatgcagatggtcgatgaattccgccattcgaccatccagatgaacctcaaaaaatggtatatggagaattatatcgatccggcgggcttcgatattaccgaggaagcattcggcaagtgctacgccacaactatcgggcgccagttcggtgagggcttcatcaccggcgacgcgatcacctcagcgaacatgtacctgaccgtcgtggccgagacagcgttcaccaacacgctgttcgtcgccatgccgtcggaggccgctcgtaacggagattacgctctgccgacggtgttcctatccgtgcagtccgatgagagccggcacgtgggtaacggccactcgatgctgatggcgatgctcaaggagcccgacaaccacctgctgcttgagcgcgacctccgttacgccttctggcagaaccacgccatcgtcgatgcggcggtcggcacgttcatcgagtacgggaccacgaaccgcgataagaacaaagagtcttacgcggaaatgtggcaccgctggatcttcgaggactactaccgcacatacatgttgccgctcgagaagtacggcatcaaggttcatcacgacgatgttcaggctgcttgggatcgcttgaccaagaagttctacgtccacgccgtcgcacagttcttttccgtcggctggccggtcaatttctggcgcatcgaagcccagacggaaaaagacttcgagtggttcgagcataagtaccccggctggtacgcgcagtttggcgagtactggaagtggtacgagaagctgtcccacaagggtcagaaggttctcctgttcaatgaggatgtcggatacgtctatccgcaccggtgctggagttctctggtgccatgcctcatccgtgaggacatcgtcgtcgacgagatcgatggcaagctgcacaccttcgctcatgagatcgaccgttggactgcagtggaagccttctcgggcgaataccaaggtaggccgacacctgcgatgggccgcttcagcggacgccgcgagtgggaagactgctaccacggctgggatcttgccgacgcgatcaaggatctcggcttcgttcggactgacggcaagacactcgtgccgcagccgcatctgcgcttcgacaacaaggagatgtggaccctcgacgatgttcgcgggcacaccctccagagcccgttgcagatgctgcgtgcgatgacgccatccgagcgtgacgcacaccttacccaataccgggctgggttctcgatcaatccttgcaactga

SEQ ID NO: 13 - DNA, 372 bp, Methylobacterium sp.
  source 1..372: mol_type = genomic DNA
atgtctgtgaatgcacgcaatgcgtcgaaccagaacatcttccaaaagatgggggatctttctttcgatcagacgatctcacaccagtgcggtgttaccatgaacgacagcgtcgaggcgcgcgcgatcgctgaattcatgggccagaagccgggcgtcaccatcacccaccagcctgcgctgatccgcatcgatggaaacggcgaactcgtcttcaagatggacgagatcagcgaaattctcggcaaggagatgaccgcggagatcttcgaggtcaacacttcgacgcactacggcaggatgatccgcatcgacgacaatacggtcacgttgttcggcgatatggaagctgctctgcagtacatcgagtaa

SEQ ID NO: 14 - DNA, 1068 bp, Methylobacterium sp.
  source 1..1068: mol_type = genomic DNA
atgacgttggaagccgccacacggaagtcgggcgcagccggctccgcgattttcgcggactcggatagccgcaagtaccggtatttcgaacccaagggcaaacgtgcgacccactacgaagacatgacggtcgacgtacagcccgatcccgagcgttacctactccagaactggatcatttcgttcgcggacgggaagggcgcgtactcgaaggagaatacggcggccctgagctccgactggcacgctttccgcgcgcctgatcaggagtgggagcgcacgcattatcagcgacagtcgaagatcgaggcgatggtgcaatccgtgatcgccaatggtcgcaaggccggcgctccggccgccttcgacaaggcctgggtgaagatcctccagaaccatctcggcgcatggaagcatgtggaattcgggctgggcaccgcgctcatgcaggcgcagcgctacggctacacgcagatgatcaacaacgcgaccctgacgaactcgtcctacaagctgcgcctcgcccaggacatcacgctctacctcgcggagatcgggatggacatcgccgggttcgacgacacggcgggtaagcggcactggctcgaggacggaacttggcagggcacccgggaagcggtggaggccatcatgggctcgaccgactacctggagcagtacttcgcgacgaacatcgtcttcgagccgctggtgggcgagctgtttcgcagcggcttcctgatgcaggtcgcgtcctcgaacggggacttcatcacgccgccggtgatctccgcggccgaggcggactacgagcgcaacctcgccaacgcgatcgacctgtttcacctgctcgccacggacgccgcgcacgccgagcacaaccggaccctgttccggagctgggtcgaccggcacgtcgctttggctcgcaaggcagctgccgatctccagccgatctggtcacagcctcattcgaagccggcccagttcgccgacgcgaaagcggcgtccaccgaacgtgtcaccaagatcctcggcgagctcggcctcgatcttcctcaggagtaa

SEQ ID NO: 15 - DNA, 1038 bp, Methylobacterium sp.
  source 1..1038: mol_type = genomic DNA
atgagcgaggtatacaccgtcaaactcaacccggtgggcgtcgagttcgaggtcgaggaagacgagaccgtcctcgacgcggccttccggcagggcattgcgctcccacacggttgcaaagaggggcgttgcgcggcttgcaaatgcatcgtcaccgacggcgaagtcgagatgctgccctattccacgttcgcgctgaacgagacggagcgtgaccagaaccatgttcttctctgccggagccttatatcgagcgatatcgaggtcgacctcctcaactacgacgaggagctgctctcaaaatcgatcccggtccgggacctccaagggcgtgtcagccgcgtctcgagtctgacgcacgacattcgcctgcttgaaatcgaacttgatcaacctctgaagttctgggctggacagtatgtcgacatcacacttccgggagcagaggagattacgcgctcgttttccatggcaaatccgccgagcgaaacgcaaaaactctcgttcatcatcaaaaaatatcccggcggccgattctcctcccgcctcgacggagagctcgacgtcggaacccccgtcggcgtcaaaggaccctacggaacctgcttccgccgtgaaaacagaggaggagccctaatcctcgttgggggtggttcggggatgtcaccgctctggtccatccttcacgaccacatcgcgagcggagaagaacgcccagtccatttcttctatggcgcgcgcacgcaaacggatctgttctatctggatctgttcgccgaggtggcgcggtccaatccgcgcttcaccttcataccggtcctctcccacgcttcggacgacgccgcgtggcagggtgagcgggggttcgtccacgaggtcgtcaacgaccatctacgtcgtctcgcttacggggaagatctcgacgtgtacgcctgcggtccggagccgatgatcgaggcactcaccccagtgctgcagatgaacgacgtatcttcggagcggattttcttcgacaagttcacaccggcacggacgtaa

SEQ ID NO: 16 - DNA, 1671 bp, Methylobacterium sp.
  source 1..1671: mol_type = genomic DNA
atgtctggaacactgacactcaacaagataacagcccagaaagggatctccgtcggagaggcggccaagaagatcgccgatctcggctggaacccctcgtacgtccaggaggctatgaccttcccaacggactacaagatcaccaaggcccccaaagatccgatgaagcaggttctgcggtcctatttcccaatgcaggaggagaaggacaatcgggtctacggggcgctcgatgcggcgctgcgcggcgacatgttccgaaacgtcgagccgcggtgggtcgagtggatgaagctcttcctggctatcattccgtttcccgagatttcggccgcccgttcgatggcgatggtcggtcggttggcgcccggcgaggagctgcgtaccgggtttaccatgcagatggtcgacgagttccgccactcgaccatccaaatgaacctcaagaaatggtacatggagaactatatcgacccggcgggcttcgacatcaccgaagaggcgttcggcaaatgctatgcgacgacgatcggtcgtcaattcggcgaaggtttcatcaccggcgacgcgatcacctcggccaacatttacctgaccgtggtcgccgagaccgccttcacgaacacgctgttcgtggccatgccgtccgaggccgcccgtaacggcgactatgctctgccgaccgtgttcctgtcggtgcagtccgacgagagccggcacgtcggcaacgggcattcgatgctgatgtcgatcctgaaagagccggaaaaccacctcctgctcgagcgcgacctgcgctacgccttctggcagaaccacgccatcgtcgatgccgcgattggcacgttcatcgagtacggcaccacgaaccgtgacaaggacaaggagtcctacgcggagatgtggcaccgctggattttcgaggactactatcgcacctacatgctcccgctggagaaatacggcatcaaaattcaccacgacgacgttcagacggcctggaagcgcatcaccgagaaacattacgttcacaagatcgcacagttcttctccgtcggctggccggtgaatttctggcggattgaggcgcagaccgagaaagacttcgagtggtttgagcacaaatatccgggttggtacgctcagttcggcgagtactggaagtggtacggaaagctgagccggcccggccagaagatcgtcaccttcaacgaggatgtcggttacgtctatccgcaccgctgctggagttcgctggtgccgtgcgtcgtgcgcgaggatctcgtcactgacgagatcgacggccagctgcacaccttcgcgcacgagatcgatcgttggaccgcggtcgaagccttcgccaacgaatacgagggacgtcccacaccggccatggggcggttcagcggccgtcgtgaatgggagagtgtataccacaactgggatctggcagatgccatcaaggatctcggcttcgtccgcacagacggcaagaccctcgttcctcaaccgcatctgcgcttcgacaacaaggatatgtggacgctcgacgatgtgcgtggatacacgctgaagagcccgctgatcaccttgcgcgaaatgtcgccggaggcgcgcgaggcgcatctcgccgaatatcgcgcgggcttcacgatcaatccctgcaactga

SEQ ID NO: 17 - protein, 279 aa, synthetic construct
  VARIANT 1..63: note = X can be any amino acid or can be absent.
  VARIANT 70..77: note = X can be any amino acid or can be absent.
  VARIANT 79..80: note = X can be any amino acid or can be absent.
  VARIANT 105..135: note = X can be any amino acid or can be absent.
  VARIANT 141..151: note = X can be any amino acid or can be absent.
  VARIANT 153: note = X can be any amino acid or can be absent.
  VARIANT 155: note = X can be any amino acid or can be absent.
  VARIANT 158..159: note = X can be any amino acid or can be absent.
  VARIANT 163: note = X can be any amino acid or can be absent.
  VARIANT 166: note = X can be any amino acid or can be absent.
  VARIANT 174: note = X can be any amino acid or can be absent.
  VARIANT 179: note = X can be any amino acid or can be absent.
  VARIANT 189: note = X can be any amino acid or can be absent.
  VARIANT 195..216: note = X can be any amino acid or can be absent.
  VARIANT 222..230: note = X can be any amino acid or can be absent.
  VARIANT 233: note = X can be any amino acid or can be absent.
  VARIANT 236..240: note = X can be any amino acid or can be absent.
  VARIANT 242..279: note = X can be any amino acid or can be absent.
  VARIANT 67: note = X can be any amino acid or can be absent.
XXXXXXXXXXXXXXXXXXXXXXXXXXXXXXXXXXXXXXXXXXXXXXXXXXXXXXXXXXXXXXXMPTXLPXXXXXXXXRXXPVRRLSWPDTARFLILVARVRLLDXXXXXXXXXXXXXXXXXXXXXXXXXXXXXXXLRLHAXXXXXXXXXXXVXRXGSXXAGDXLLXLMRRWLAXHEAIXALLPGVPEPXHVAQVXXXXXXXXXXXXXXXXXXXXXXRAILQXXXXXXXXXVPXSRXXXXXPXXXXXXXXXXXXXXXXXXXXXXXXXXXXXXXXXXXXXX

SEQ ID NO: 18 - protein, 154 aa, synthetic construct
  VARIANT 1..7: note = X can be any amino acid or can be absent.
  VARIANT 9..10: note = X can be any amino acid or can be absent.
  VARIANT 19: note = X can be any amino acid or can be absent.
  VARIANT 52: note = X can be any amino acid or can be absent.
  VARIANT 71: note = X can be any amino acid or can be absent.
  VARIANT 74: note = X can be any amino acid or can be absent.
  VARIANT 94: note = X can be any amino acid or can be absent.
  VARIANT 96..154: note = X can be any amino acid or can be absent.
XXXXXXXMXXPLRRTVQVXEDGRMNLPADMRRVLGLTGAGRVILTQDEDGIXITTAEQALKRVRSLAAPFXRGXGSVVDEFIAERRADAAREDXEXXXXXXXXXXXXXXXXXXXXXXXXXXXXXXXXXXXXXXXXXXXXXXXXXXXXXXXXXXX

SEQ ID NO: 19 - protein, 103 aa, synthetic construct
  VARIANT 2..29: note = X can be any amino acid or can be absent.
  VARIANT 42: note = X can be any amino acid or can be absent.
  VARIANT 69..72: note = X can be any amino acid or can be absent.
  VARIANT 77..103: note = X can be any amino acid or can be absent.
MXXXXXXXXXXXXXXXXXXXXXXXXXXXXPQSYALQILAIAXAMSVLGLGGVWIASRIYDRNTRRLEAXXXXRRGDXXXXXXXXXXXXXXXXXXXXXXXXXXX

SEQ ID NO: 20 - protein, 2503 aa, synthetic construct
  VARIANT 1..7: note = X can be any amino acid or can be absent.
  VARIANT 12..27: note = X can be any amino acid or can be absent.
  VARIANT 29..33: note = X can be any amino acid or can be absent.
  VARIANT 39: note = X can be any amino acid or can be absent.
  VARIANT 42: note = X can be any amino acid or can be absent.
  VARIANT 46..47: note = X can be any amino acid or can be absent.
  VARIANT 50: note = X can be any amino acid or can be absent.
  VARIANT 58: note = X can be any amino acid or can be absent.
  VARIANT 60: note = X can be any amino acid or can be absent.
  VARIANT 62..63: note = X can be any amino acid or can be absent.
  VARIANT 65: note = X can be any amino acid or can be absent.
  VARIANT 71: note = X can be any amino acid or can be absent.
  VARIANT 73: note = X can be any amino acid or can be absent.
  VARIANT 75: note = X can be any amino acid or can be absent.
  VARIANT 77: note = X can be any amino acid or can be absent.
  VARIANT 79: note = X can be any amino acid or can be absent.
  VARIANT 81: note = X can be any amino acid or can be absent.
  VARIANT 87: note = X can be any amino acid or can be absent.
  VARIANT 89: note = X can be any amino acid or can be absent.
  VARIANT 92: note = X can be any amino acid or can be absent.
  VARIANT 95..98: note = X can be any amino acid or can be absent.
  VARIANT 101: note = X can be any amino acid or can be absent.
  VARIANT 108: note = X can be any amino acid or can be absent.
  VARIANT 112..116: note = X can be any amino acid or can be absent.
  VARIANT 119..120: note = X can be any amino acid or can be absent.
  VARIANT 123..126: note = X can be any amino acid or can be absent.
  VARIANT 130: note = X can be any amino acid or can be absent.
  VARIANT 132..133: note = X can be any amino acid or can be absent.
  VARIANT 135..142: note = X can be any amino acid or can be absent.
  VARIANT 144: note = X can be any amino acid or can be absent.
  VARIANT 148..149: note = X can be any amino acid or can be absent.
  VARIANT 162..163: note = X can be any amino acid or can be absent.
  VARIANT 182: note = X can be any amino acid or can be absent.
  VARIANT 186: note = X can be any amino acid or can be absent.
  VARIANT 189: note = X can be any amino acid or can be absent.
  VARIANT 192: note = X can be any amino acid or can be absent.
  VARIANT 195: note = X can be any amino acid or can be absent.
  VARIANT 197: note = X can be any amino acid or can be absent.
  VARIANT 200..201: note = X can be any amino acid or can be absent.
  VARIANT 205: note = X can be any amino acid or can be absent.
  VARIANT 220: note = X can be any amino acid or can be absent.
  VARIANT 222..226: note = X can be any amino acid or can be absent.
  VARIANT 230..241: note = X can be any amino acid or can be absent.
  VARIANT 244..249: note = X can be any amino acid or can be absent.
  VARIANT 266: note = X can be any amino acid or can be absent.
  VARIANT 268..269: note = X can be any amino acid or can be absent.
  VARIANT 276: note = X can be any amino acid or can be absent.
  VARIANT 289: note = X can be any amino acid or can be absent.
  VARIANT 302..303: note = X can be any amino acid or can be absent.
  VARIANT 306: note = X can be any amino acid or can be absent.
  VARIANT 312: note = X can be any amino acid or can be absent.
  VARIANT 315..321: note = X can be any amino acid or can be absent.
  VARIANT 324: note = X can be any amino acid or can be absent.
  VARIANT 326: note = X can be any amino acid or can be absent.
  VARIANT 336..347: note = X can be any amino acid or can be absent.
  VARIANT 358..366: note = X can be any amino acid or can be absent.
  VARIANT 370..371: note = X can be any amino acid or can be absent.
  VARIANT 373..374: note = X can be any amino acid or can be absent.
  VARIANT 376..378: note = X can be any amino acid or can be absent.
  VARIANT 381..382: note = X can be any amino acid or can be absent.
  VARIANT 385: note = X can be any amino acid or can be absent.
  VARIANT 388: note = X can be any amino acid or can be absent.
  VARIANT 390: note = X can be any amino acid or can be absent.
  VARIANT 394: note = X can be any amino acid or can be absent.
  VARIANT 400: note = X can be any amino acid or can be absent.
  VARIANT 402..403: note = X can be any amino acid or can be absent.
  VARIANT 406: note = X can be any amino acid or can be absent.
  VARIANT 410: note = X can be any amino acid or can be absent.
  VARIANT 413..420: note = X can be any amino acid or can be absent.
  VARIANT 423..430: note = X can be any amino acid or can be absent.
  VARIANT 432..434: note = X can be any amino acid or can be absent.
  VARIANT 436: note = X can be any amino acid or can be absent.
  VARIANT 438..446: note = X can be any amino acid or can be absent.
  VARIANT 452: note = X can be any amino acid or can be absent.
  VARIANT 455..459: note = X can be any amino acid or can be absent.
  VARIANT 462..467: note = X can be any amino acid or can be absent.
  VARIANT 469..470: note = X can be any amino acid or can be absent.
  VARIANT 474: note = X can be any amino acid or can be absent.
  VARIANT 477: note = X can be any amino acid or can be absent.
  VARIANT 503: note = X can be any amino acid or can be absent.
  VARIANT 508: note = X can be any amino acid or can be absent.
  VARIANT 511: note = X can be any amino acid or can be absent.
  VARIANT 513: note = X can be any amino acid or can be absent.
  VARIANT 516..517: note = X can be any amino acid or can be absent.
  VARIANT 521..522: note = X can be any amino acid or can be absent.
  VARIANT 525..529: note = X can be any amino acid or can be absent.
  VARIANT 531..557: note = X can be any amino acid or can be absent.
  VARIANT 559..561: note = X can be any amino acid or can be absent.
  VARIANT 563: note = X can be any amino acid or can be absent.
  VARIANT 566..583: note = X can be any amino acid or can be absent.
  VARIANT 587: note = X can be any amino acid or can be absent.
  VARIANT 590: note = X can be any amino acid or can be absent.
  VARIANT 598..599: note = X can be any amino acid or can be absent.
  VARIANT 612: note = X can be any amino acid or can be absent.
  VARIANT 620..621: note = X can be any amino acid or can be absent.
  VARIANT 623..627: note = X can be any amino acid or can be absent.
  VARIANT 636: note = X can be any amino acid or can be absent.
  VARIANT 639: note = X can be any amino acid or can be absent.
  VARIANT 650..651: note = X can be any amino acid or can be absent.
  VARIANT 656: note = X can be any amino acid or can be absent.
  VARIANT 671: note = X can be any amino acid or can be absent.
  VARIANT 673: note = X can be any amino acid or can be absent.
  VARIANT 677..680: note = X can be any amino acid or can be absent.
  VARIANT 683: note = X can be any amino acid or can be absent.
  VARIANT 685: note = X can be any amino acid or can be absent.
  VARIANT 692: note = X can be any amino acid or can be absent.
  VARIANT 710..711: note = X can be any amino acid or can be absent.
  VARIANT 730: note = X can be any amino acid or can be absent.
  VARIANT 733: note = X can be any amino acid or can be absent.
  VARIANT 751: note = X can be any amino acid or can be absent.
  VARIANT 757..759: note = X can be any amino acid or can be absent.
  VARIANT 761: note = X can be any amino acid or can be absent.
  VARIANT 764: note = X can be any amino acid or can be absent.
  VARIANT 766: note = X can be any amino acid or can be absent.
  VARIANT 770: note = X can be any amino acid or can be absent.
  VARIANT 775..776: note = X can be any amino acid or can be absent.
  VARIANT 779..783: note = X can be any amino acid or can be absent.
  VARIANT 786: note = X can be any amino acid or can be absent.
  VARIANT 816..825: note = X can be any amino acid or can be absent.
  VARIANT 829: note = X can be any amino acid or can be absent.
  VARIANT 838..839: note = X can be any amino acid or can be absent.
  VARIANT 857..858: note = X can be any amino acid or can be absent.
  VARIANT 863..864: note = X can be any amino acid or can be absent.
  VARIANT 869..870: note = X can be any amino acid or can be absent.
  VARIANT 877: note = X can be any amino acid or can be absent.
  VARIANT 883: note = X can be any amino acid or can be absent.
  VARIANT 886: note = X can be any amino acid or can be absent.
  VARIANT 893..894: note = X can be any amino acid or can be absent.
  VARIANT 896..902: note = X can be any amino acid or can be absent.
  VARIANT 905: note = X can be any amino acid or can be absent.
  VARIANT 907..908: note = X can be any amino acid or can be absent.
  VARIANT 911..913: note = X can be any amino acid or can be absent.
  VARIANT 916: note = X can be any amino acid or can be absent.
  VARIANT 922: note = X can be any amino acid or can be absent.
  VARIANT 926..927: note = X can be any amino acid or can be absent.
  VARIANT 929: note = X can be any amino acid or can be absent.
  VARIANT 936: note = X can be any amino acid or can be absent.
  VARIANT 938: note = X can be any amino acid or can be absent.
  VARIANT 944..947: note = X can be any amino acid or can be absent.
  VARIANT 949..952: note = X can be any amino acid or can be absent.
  VARIANT 959..970: note = X can be any amino acid or can be absent.
  VARIANT 973..977: note = X can be any amino acid or can be absent.
  VARIANT 980: note = X can be any amino acid or can be absent.
  VARIANT 982: note = X can be any amino acid or can be absent.
  VARIANT 985..989: note = X can be any amino acid or can be absent.
  VARIANT 991..993: note = X can be any amino acid or can be absent.
  VARIANT 1006: note = X can be any amino acid or can be absent.
  VARIANT 1008..1009: note = X can be any amino acid or can be absent.
  VARIANT 1016: note = X can be any amino acid or can be absent.
  VARIANT 1019..1020: note = X can be any amino acid or can be absent.
  VARIANT 1023..1024: note = X can be any amino acid or can be absent.
  VARIANT 1026: note = X can be any amino acid or can be absent.
  VARIANT 1030..1034: note = X can be any amino acid or can be absent.
  VARIANT 1038..1039: note = X can be any amino acid or can be absent.
  VARIANT 1046..1047: note = X can be any amino acid or can be absent.
  VARIANT 1049..1050: note = X can be any amino acid or can be absent.
  VARIANT 1053: note = X can be any amino acid or can be absent.
  VARIANT 1055..1056: note = X can be any amino acid or can be absent.
  VARIANT 1058: note = X can be any amino acid or can be absent.
  VARIANT 1060..1061: note = X can be any amino acid or can be absent.
  VARIANT 1063..1065: note = X can be any amino acid or can be absent.
  VARIANT 1067: note = X can be any amino acid or can be absent.
  VARIANT 1069: note = X can be any amino acid or can be absent.
  VARIANT 1073: note = X can be any amino acid or can be absent.
  VARIANT 1076: note = X can be any amino acid or can be absent.
  VARIANT 1079..1080: note = X can be any amino acid or can be absent.
  VARIANT 1082..1084: note = X can be any amino acid or can be absent.
  VARIANT 1090: note = X can be any amino acid or can be absent.
  VARIANT 1093..1095: note = X can be any amino acid or can be absent.
  VARIANT 1097..1099: note = X can be any amino acid or can be absent.
  VARIANT 1103..1104: note = X can be any amino acid or can be absent.
  VARIANT 1107..1109: note = X can be any amino acid or can be absent.
  VARIANT 1111..1112: note = X can be any amino acid or can be absent.
  VARIANT 1114: note = X can be any amino acid or can be absent.
  VARIANT 1116..1118: note = X can be any amino acid or can be absent.
  VARIANT 1121..1122: note = X can be any amino acid or can be absent.
  VARIANT 1128..1129: note = X can be any amino acid or can be absent.
  VARIANT 1132: note = X can be any amino acid or can be absent.
  VARIANT 1136: note = X can be any amino acid or can be absent.
  VARIANT 1140: note = X can be any amino acid or can be absent.
  VARIANT 1143: note = X can be any amino acid or can be absent.
  VARIANT 1146..1148: note = X can be any amino acid or can be absent.
  VARIANT 1150: note = X can be any amino acid or can be absent.
  VARIANT 1156..1157: note = X can be any amino acid or can be absent.
  VARIANT 1159: note = X can be any amino acid or can be absent.
  VARIANT 1166..1167: note = X can be any amino acid or can be absent.
  VARIANT 1169: note = X can be any amino acid or can be absent.
  VARIANT 1172..1191: note = X can be any amino acid or can be absent.
  VARIANT 1193: note = X can be any amino acid or can be absent.
  VARIANT 1195..1197: note = X can be any amino acid or can be absent.
  VARIANT 1199: note = X can be any amino acid or can be absent.
  VARIANT 1202..1204: note = X can be any amino acid or can be absent.
  VARIANT 1206..1209: note = X can be any amino acid or can be absent.
  VARIANT 1214..1215: note = X can be any amino acid or can be absent.
  VARIANT 1218..1219: note = X can be any amino acid or can be absent.
  VARIANT 1221: note = X can be any amino acid or can be absent.
  VARIANT 1223: note = X can be any amino acid or can be absent.
  VARIANT 1228: note = X can be any amino acid or can be absent.
  VARIANT 1231..1232: note = X can be any amino acid or can be absent.
  VARIANT 1235..1236: note = X can be any amino acid or can be absent.
  VARIANT 1238: note = X can be any amino acid or can be absent.
  VARIANT 1241..1243: note = X can be any amino acid or can be absent.
  VARIANT 1245: note = X can be any amino acid or can be absent.
  VARIANT 1247: note = X can be any amino acid or can be absent.
  VARIANT 1250..1251: note = X can be any amino acid or can be absent.
  VARIANT 1253..1258: note = X can be any amino acid or can be absent.
  VARIANT 1261: note = X can be any amino acid or can be absent.
  VARIANT 1263..1265: note = X can be any amino acid or can be absent.
  VARIANT 1267..1277: note = X can be any amino acid or can be absent.
  VARIANT 1280: note = X can be any amino acid or can be absent.
  VARIANT 1283: note = X can be any amino acid or can be absent.
  VARIANT 1285..1287: note = X can be any amino acid or can be absent.
  VARIANT 1289..1291: note = X can be any amino acid or can be absent.
  VARIANT 1293..1294: note = X can be any amino acid or can be absent.
  VARIANT 1301: note = X can be any amino acid or can be absent.
  VARIANT 1305..1313: note = X can be any amino acid or can be absent.
  VARIANT 1319: note = X can be any amino acid or can be absent.
  VARIANT 1322..1325: note = X can be any amino acid or can be absent.
  VARIANT 1328..1329: note = X can be any amino acid or can be absent.
  VARIANT 1331..1452: note = X can be any amino acid or can be absent.
  VARIANT 1454: note = X can be any amino acid or can be absent.
  VARIANT 1457..1458: note = X can be any amino acid or can be absent.
  VARIANT 1464: note = X can be any amino acid or can be absent.
  VARIANT 1467..1469: note = X can be any amino acid or can be absent.
  VARIANT 1471..1472: note = X can be any amino acid or can be absent.
  VARIANT 1476: note = X can be any amino acid or can be absent.
  VARIANT 1478: note = X can be any amino acid or can be absent.
  VARIANT 1482: note = X can be any amino acid or can be absent.
  VARIANT 1485..1486: note = X can be any amino acid or can be absent.
  VARIANT 1488: note = X can be any amino acid or can be absent.
  VARIANT 1493: note = X can be any amino acid or can be absent.
  VARIANT 1497: note = X can be any amino acid or can be absent.
  VARIANT 1501: note = X can be any amino acid or can be absent.
  VARIANT 1503: note = X can be any amino acid or can be absent.
  VARIANT 1505..1506: note = X can be any amino acid or can be absent.
  VARIANT 1508..1511: note = X can be any amino acid or can be absent.
  VARIANT 1515: note = X can be any amino acid or can be absent.
  VARIANT 1524..1526: note = X can be any amino acid or can be absent.
  VARIANT 1530..1531: note = X can be any amino acid or can be absent.
  VARIANT 1534..1538: note = X can be any amino acid or can be absent.
  VARIANT 1541: note = X can be any amino acid or can be absent.
  VARIANT 1550: note = X can be any amino acid or can be absent.
  VARIANT 1552..1553: note = X can be any amino acid or can be absent.
  VARIANT 1560: note = X can be any amino acid or can be absent.
  VARIANT 1563..1565: note = X can be any amino acid or can be absent.
  VARIANT 1570..1576: note = X can be any amino acid or can be absent.
  VARIANT 1583..1589: note = X can be any amino acid or can be absent.
  VARIANT 1591: note = X can be any amino acid or can be absent.
  VARIANT 1595: note = X can be any amino acid or can be absent.
  VARIANT 1604: note = X can be any amino acid or can be absent.
  VARIANT 1607..1611: note = X can be any amino acid or can be absent.
  VARIANT 1615: note = X can be any amino acid or can be absent.
  VARIANT 1625: note = X can be any amino acid or can be absent.
  VARIANT 1635..1637: note = X can be any amino acid or can be absent.
  VARIANT 1652: note = X can be any amino acid or can be absent.
  VARIANT 1654: note = X can be any amino acid or can be absent.
  VARIANT 1656: note = X can be any amino acid or can be absent.
  VARIANT 1708..1711: note = X can be any amino acid or can be absent.
  VARIANT 1714: note = X can be any amino acid or can be absent.
  VARIANT 1735: note = X can be any amino acid or can be absent.
  VARIANT 1737..1741: note = X can be any amino acid or can be absent.
  VARIANT 1757: note = X can be any amino acid or can be absent.
  VARIANT 1761: note = X can be any amino acid or can be absent.
  VARIANT 1763: note = X can be any amino acid or can be absent.
  VARIANT 1775: note = X can be any amino acid or can be absent.
  VARIANT 1781..1782: note = X can be any amino acid or can be absent.
  VARIANT 1784: note = X can be any amino acid or can be absent.
  VARIANT 1792..1793: note = X can be any amino acid or can be absent.
  VARIANT 1809: note = X can be any amino acid or can be absent.
  VARIANT 1812: note = X can be any amino acid or can be absent.
  VARIANT 1815..1823: note = X can be any amino acid or can be absent.
  VARIANT 1827: note = X can be any amino acid or can be absent.
  VARIANT 1829: note = X can be any amino acid or can be absent.
  VARIANT 1851: note = X can be any amino acid or can be absent.
  VARIANT 1855..1858: note = X can be any amino acid or can be absent.
  VARIANT 1863: note = X can be any amino acid or can be absent.
  VARIANT 1867..1869: note = X can be any amino acid or can be absent.
  VARIANT 1871: note = X can be any amino acid or can be absent.
  VARIANT 1881: note = X can be any amino acid or can be absent.
  VARIANT 1896: note = X can be any amino acid or can be absent.
  VARIANT 1899..1900: note = X can be any amino acid or can be absent.
  VARIANT 1902: note = X can be any amino acid or can be absent.
  VARIANT 1906..1909: note = X can be any amino acid or can be absent.
  VARIANT 1920..1922: note = X can be any amino acid or can be absent.
  VARIANT 1944: note = X can be any amino acid or can be absent.
  VARIANT 1952..1953: note = X can be any amino acid or can be absent.
  VARIANT 1965: note = X can be any amino acid or can be absent.
  VARIANT 1969..1970: note = X can be any amino acid or can be absent.
  VARIANT 1973: note = X can be any amino acid or can be absent.
  VARIANT 1976..1977: note = X can be any amino acid or can be absent.
  VARIANT 1979: note = X can be any amino acid or can be absent.
  VARIANT 1992: note = X can be any amino acid or can be absent.
  VARIANT 1997..2004: note = X can be any amino acid or can be absent.
  VARIANT 2007: note = X can be any amino acid or can be absent.
  VARIANT 2012: note = X can be any amino acid or can be absent.
  VARIANT 2014..2015: note = X can be any amino acid or can be absent.
  VARIANT 2021: note = X can be any amino acid or can be absent.
  VARIANT 2049..2051: note = X can be any amino acid or can be absent.
  VARIANT 2053: note = X can be any amino acid or can be absent.
  VARIANT 2066: note = X can be any amino acid or can be absent.
  VARIANT 2076: note = X can be any amino acid or can be absent.
  VARIANT 2085: note = X can be any amino acid or can be absent.
  VARIANT 2087: note = X can be any amino acid or can be absent.
  VARIANT 2092: note = X can be any amino acid or can be absent.
  VARIANT 2096..2097: note = X can be any amino acid or can be absent.
  VARIANT 2100: note = X can be any amino acid or can be absent.
  VARIANT 2109..2165: note = X can be any amino acid or can be absent.
  VARIANT 2167..2178: note = X can be any amino acid or can be absent.
  VARIANT 2180..2186: note = X can be any amino acid or can be absent.
  VARIANT 2188..2191: note = X can be any amino acid or can be absent.
  VARIANT 2193..2209: note = X can be any amino acid or can be absent.
  VARIANT 2211..2212: note = X can be any amino acid or can be absent.
  VARIANT 2217..2218: note = X can be any amino acid or can be absent.
  VARIANT 2220..2222: note = X can be any amino acid or can be absent.
  VARIANT 2224..2225: note = X can be any amino acid or can be absent.
  VARIANT 2230..2231: note = X can be any amino acid or can be absent.
  VARIANT 2233..2235: note = X can be any amino acid or can be absent.
  VARIANT 2240: note = X can be any amino acid or can be absent.
  VARIANT 2242: note = X can be any amino acid or can be absent.
  VARIANT 2245..2246: note = X can be any amino acid or can be absent.
  VARIANT 2252: note = X can be any amino acid or can be absent.
  VARIANT 2256..2257: note = X can be any amino acid or can be absent.
  VARIANT 2260: note = X can be any amino acid or can be absent.
  VARIANT 2265..2266: note = X can be any amino acid or can be absent.
  VARIANT 2270..2271: note = X can be any amino acid or can be absent.
  VARIANT 2275..2276: note = X can be any amino acid or can be absent.
  VARIANT 2278..2279: note = X can be any amino acid or can be absent.
  VARIANT 2283..2286: note = X can be any amino acid or can be absent.
  VARIANT 2288: note = X can be any amino acid or can be absent.
  VARIANT 2290..2291: note = X can be any amino acid or can be absent.
  VARIANT 2298: note = X can be any amino acid or can be absent.
  VARIANT 2300..2301: note = X can be any amino acid or can be absent.
  VARIANT 2303..2308: note = X can be any amino acid or can be absent.
  VARIANT 2313..2322: note = X can be any amino acid or can be absent.
  VARIANT 2325..2327: note = X can be any amino acid or can be absent.
  VARIANT 2329: note = X can be any amino acid or can be absent.
  VARIANT 2332: note = X can be any amino acid or can be absent.
  VARIANT 2335: note = X can be any amino acid or can be absent.
  VARIANT 2340: note = X can be any amino acid or can be absent.
  VARIANT 2342..2353: note = X can be any amino acid or can be absent.
  VARIANT 2356: note = X can be any amino acid or can be absent.
  VARIANT 2359..2367: note = X can be any amino acid or can be absent.
  VARIANT 2370: note = X can be any amino acid or can be absent.
  VARIANT 2373..2374: note = X can be any amino acid or can be absent.
  VARIANT 2376..2406: note = X can be any amino acid or can be absent.
  VARIANT 2414: note = X can be any amino acid or can be absent.
  VARIANT 2416: note = X can be any amino acid or can be absent.
  VARIANT 2418: note = X can be any amino acid or can be absent.
  VARIANT 2423..2503: note = X can be any amino acid or can be absent.
  VARIANT 954: note = X can be any amino acid or can be absent.
  VARIANT 956: note = X can be any amino acid or can be absent.
  VARIANT 996: note = X can be any amino acid or can be absent.
  VARIANT 998: note = X can be any amino acid or can be absent.
XXXXXXXDTLEXXXXXXXXXXXXXXXXRXXXXXLACTVXDHXSIAXXQNXVPIIRDIXLXNXXDXDLADVXLXIXAXPXLXRPLTLXIXRIXAGXXXXIDXPDLRIDXAILXXXXXAGXXESXXXXVTLXLXXSXXXXXXXXEXAREXXDLRLLPPSHWGGXXAAPELLAAFVRPNDPAVDXILRXAAXILXRAXRXTAXXDGYXSGRKARAWEMAEAIXAXXXXXAMAXXXXXXXXXXXXRIXXXXXXYVLPPASFERSGQKVRXPXXIVERRLXTCLDLTLLWAACXEQAGLNPLLVLTXXHAXLGLWLXDEXXXXXXXDDXQXLRKRRDLQEXXXXXXXXXXXXLILIETTILTXXXXXXXXXDPPXXFXXAXXXGAXXIDXDAXAXLEMXLDLRRXRXXGIXPLDXGEXXXXXXXXAPXXXXXXXXLXXXQXLXXXXXXXXXAPPSFXEDXXXXXIDXXXXXXPXXRLEXWKXRLLDLTLRNKLLNFKPGKGSLTLDCXEPGAXEDXLXAGXXFRLXXRPXXXXXDXXXXXXXXXXXXXXXXXXXXXXXXXXXAXXXRXEIXXXXXXXXXXXXXXXXXXELEXRLXDLFRLARXXFEEGGANVLFLAXGFLTWTRXXGXXXXXRAPLLLVPXALXRASVRAGFRLXXHDEEXRLNPTLLEMLRQDFXLXMPDXXXXLPXDXSGIDVEXIWRIVRTHIRDLKGWEVXXEVVLSAFSFTKFLMWKDLXERXDLLKRSPVVRHLLDTPKXAYGDGXXXTXFPXPXRLDXEHPPXXIFXXXXXPLXADSSQLSAILAAASGKDFVLFGPPGTGKSXXXXXXXXXXQTIXNMIAQCLAXXGRTVLFVSQKSAALEVVXXRRRLXXVGLGXXCLEVHAXKAQKTXVIXQLREAWXXRXXXXXXXWDXAXXDLXXXREXLNGVVXSLHXXRXNGLSAHXAXGRVIAXXXXGXXXXLXLXWPXXXXXXXXXXXXSLXXXXXRAXCXELXXXXXLXXXVGXIXDHPLRGIXAXXWSPLWRXEMXXAIXXLXRTLXXXXXSGQXXAEAMGLXXLXXTYXGXXRXLXXLXXXLXRXEARXGLXFLXXGXXXLRQAVXARXXXQXXXARLXXRLXXXYXXPXVXXXDLXXLLAEWXXAKXSNFXLRGXRLXRVXXXLXPFAQGXXPXDIGPDLXXLXEIXXXXXXXXXXXXXXXXXXXXVXEXXXAXLGXXXPXXXXWSDPXXPAXXFXAXMAWAXRLXXVIXXMXPLXXXGXDXVRXXLXXXXXXLDXEXXXLXXXXXXXXXXXGGXLAXAXXXFXXXRXXAVKAIEXLGRXXXXXXXXXLAGRAXPDXXXXPVXXEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXDXWVXXTLAVAXRWXXXLXXKAQXWXAWQXAAXXAXKAGLXPLVXAIEXGXIXXDXXXXAFEXAYARWWIDXXXTDDXXLRXXXXXFMXQRHEEAIRXFXXADSRLSXLAXXXVRARXXXXXXXIGGGVPXXXXXXXAXAFGXDPEWGTLAXEIXXXXXTKRXRHMPLRQLFXRMPNALTRLXXXTPCLMMSPLSIAQYXPXEXKPFDIVIFDEASQIAPWDAIGAIARGRQVVIVGDPEQLPPTNVGDRGVDEIXXXXDGXDVADQESILDECLAANLPQRXLXXXXXWHYRSRHESLIAFSNXHYYXGXLVTFPSPVTDDXRAVRLXXVXDGLYERGXXRVNRPEARALVAEVVXRLXDPXXXXXXXXXAFAXEXRSLGIVTFNGEQQRLIENLLDXERRXXXXPELEXFFDXXXWXEPVFVKNLEXVQGDERDAILFSVAXGPXXDXTGRXXXXISSLNREGGHXXXRRLNVAITRARRELVVFASMRXDQVDLGRXXARGVRDFKHFLXFAEXXGAXALXXAXAPTGGDIESPFEXAVMAXXXXXXXXALXARGWXIXXQVGVSXFRIDLGIVHPDAPGRYLAGVECDGATYXXXHXAATARDRDRLREXVLTDLGWRIXRVWSTDWWXDXQGALXRLDXXLRXDLDADRAKXXXXXXXXXXXXXXXXXXXXXXXXXXXXXXXXXXXXXXXXXXXXXXXXXXXXXXXXXPXXXXXXXXXXXXPXXXXXXXQXXXXPXXXXXXXXXXXXXXXXXYXXADLSXXGXXXDXXRFHDXXYXXXLAAMXAXVVXXEGPVFXDILXXRLXRAHGXXRITXXLRQXXLXXVDPXXXXTXEXXRIVLWPXGXXPXXXXXXFRPAXXXXXXXXXXRAXXXDXPLXELXGLARXLXXXXXXXXXXXXMAXRLXXXXXXXXXGLXRMXXAXXXXXXXXXXXXXXXXXXXXXXXXXXXXXXXRARFAEAXAXLXARESXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 21 - protein, 409 aa, synthetic construct
  VARIANT 1..84: note = X can be any amino acid or can be absent.
  VARIANT 105: note = X can be any amino acid or can be absent.
  VARIANT 115: note = X can be any amino acid or can be absent.
  VARIANT 130: note = X can be any amino acid or can be absent.
  VARIANT 132: note = X can be any amino acid or can be absent.
  VARIANT 143..144: note = X can be any amino acid or can be absent.
  VARIANT 153..159: note = X can be any amino acid or can be absent.
  VARIANT 173..208: note = X can be any amino acid or can be absent.
  VARIANT 220..226: note = X can be any amino acid or can be absent.
  VARIANT 238..247: note = X can be any amino acid or can be absent.
  VARIANT 260: note = X can be any amino acid or can be absent.
  VARIANT 267..269: note = X can be any amino acid or can be absent.
  VARIANT 285: note = X can be any amino acid or can be absent.
  VARIANT 298: note = X can be any amino acid or can be absent.
  VARIANT 300..301: note = X can be any amino acid or can be absent.
  VARIANT 303: note = X can be any amino acid or can be absent.
  VARIANT 305..306: note = X can be any amino acid or can be absent.
  VARIANT 320..322: note = X can be any amino acid or can be absent.
  VARIANT 324: note = X can be any amino acid or can be absent.
  VARIANT 327..328: note = X can be any amino acid or can be absent.
  VARIANT 330: note = X can be any amino acid or can be absent.
  VARIANT 333..335: note = X can be any amino acid or can be absent.
  VARIANT 349: note = X can be any amino acid or can be absent.
  VARIANT 368..382: note = X can be any amino acid or can be absent.
  VARIANT 386..409: note = X can be any amino acid or can be absent.
  VARIANT 117: note = X can be any amino acid or can be absent.
XXXXXXXXXXXXXXXXXXXXXXXXXXXXXXXXXXXXXXXXXXXXXXXXXXXXXXXXXXXXXXXXXXXXXXXXXXXXXXXXXXXXMQTILYARVSTADQTIAHQRXQAEAAGFKIXDXVVADEGVSGVSTXLXDRPQGRRLFDXXMLRRGDVLXXXXXXXVVRWVDRLGRNYAXXXXXXXXXXXXXXXXXXXXXXXXXXXXXXXXXXXXDVTETIREFMRXXXXXXXRGVIVRTVINNXXXXXXXXXXMTFDGATTDPMQXAVRDALXXXIGFMAATAQAQAEATXKEAQKAGIEHAKXRXXEXDXXAYRGRKPSYTREQXXXDXVRXXLXQGXXXVSAIAKATGLSRQXTVYRIRDNPAEAEAALARXXXXXXXXXXXXXXXWAAXXXXXXXXXXXXXXXXXXXXXXXX

SEQ ID NO: 22 - protein, 218 aa, synthetic construct
  VARIANT 2..19: note = X can be any amino acid or can be absent.
  VARIANT 23: note = X can be any amino acid or can be absent.
  VARIANT 25..26: note = X can be any amino acid or can be absent.
  VARIANT 47: note = X can be any amino acid or can be absent.
  VARIANT 51..54: note = X can be any amino acid or can be absent.
  VARIANT 56..60: note = X can be any amino acid or can be absent.
  VARIANT 62: note = X can be any amino acid or can be absent.
  VARIANT 74..130: note = X can be any amino acid or can be absent.
  VARIANT 136..140: note = X can be any amino acid or can be absent.
  VARIANT 144..145: note = X can be any amino acid or can be absent.
  VARIANT 147..161: note = X can be any amino acid or can be absent.
  VARIANT 163: note = X can be any amino acid or can be absent.
  VARIANT 166..218: note = X can be any amino acid or can be absent.
MXXXXXXXXXXXXXXXXXXYDDXIXXADAAAGEERDAIMRALAEDMXEASXXXXRXXXXXGXFVRAERPADLAXXXXXXXXXXXXXXXXXXXXXXXXXXXXXXXXXXXXXXXXXXXXXXXXXXXXXXXXXRALGRXXXXXDRRXXQXXXXXXXXXXXXXXXRXASXXXXXXXXXXXXXXXXXXXXXXXXXXXXXXXXXXXXXXXXXXXXXXXXXXXXX

SEQ ID NO: 23 - protein, 77 aa, synthetic construct
  VARIANT 1..17: note = X can be any amino acid or can be absent.
  VARIANT 21..22: note = X can be any amino acid or can be absent.
  VARIANT 40: note = X can be any amino acid or can be absent.
  VARIANT 50..51: note = X can be any amino acid or can be absent.
  VARIANT 67: note = X can be any amino acid or can be absent.
  VARIANT 72..73: note = X can be any amino acid or can be absent.
XXXXXXXXXXXXXXXXXMPVXXGIGIGRGDPLRPAVTRTXRFSGPEGFHXXPGALWLAAAAPLLATXLLLLXXRLAA

SEQ ID NO: 24 - protein, 101 aa, Methylobacterium sp.
MPTAIPIRPAPERCLSWPDTARLLILVARVRILDLEMHTVVRHGSGFADDRLLHLMRRWLAQHEAISALLPGVAEPRHVAEVRAILQVPNSRPEPEDRRAL

SEQ ID NO: 25 - protein, 83 aa, Methylobacterium sp.
MPQRRTIQVTEDGRMNLPADIRRVLGLNGAGRIVLMQDEDGIHLTTAEDPLRRVRELAAPFRRGSGSVVDEFIAERRADSGED

SEQ ID NO: 26 - protein, 48 aa, Methylobacterium sp.
MPLDYALQITAIAFGLSVLGLGGAFIASRVYDRNTRRYDEAAQLHKAD

SEQ ID NO: 27 - protein, 1982 aa, Methylobacterium sp.
VQDGIQITCSVTEHVSLAYHENAVPVIREVVVENTSEQELSDVRVRIESRPAVVQPLTLRIDRIPAGSNHHIELPDVRLDAALLAGFTEASRLELTVIVEDAAGERARHLEELRVLPPSHWGGGRSAPELLAAFVRPNDPAVDVVLRDAATKLGEAGRETGLNGYTTAKKSRAWELAEAIWAAIADRRIAYVLPPASFERAGQKVRGPSDVLERKVGTCLDLSLLYAACLEQAGLNPVLVLTVGHAFVGVWLQDDDFASATVDDMQLLRKRRDLQDLVFVETTLLTPEPPATFKVATTQGGVQVEDEAPAALEIAIDVRRCRRRGIRPMDLGDGKPTGIAPAPTIPLNQTLSAPPSFEEEARAPVDEAPETPVGRVERWKRKLLDLTLRNKLLNFKPGKGSVSLECASPGALEDGLAAGTEYRLKPLSDVLTGSDERSADLYARRHHDDGRRSYLEAALARKEIYTTSTEADLDRRLLDLYRLARNGFEEGGANILFLAVGFLSWTKKEGEAAYRAPLLLVPVTLKRSSVRAGFKLALHDDEVRINPTLLEMLREDFKLRMPELEGDLPRDGSGYDVDGIFRIVRQHVKELRGWEVVPDVVLSAFSFTKYLMWKDLVDRAEVLKRNPVVRHLIDTPKHSYGDGTPFPEPTRLDREHPPETVFAPLSADSSQLSAVLAAAGGKDFVLFGPPGTGKSQTIGNMIAQCLAQGRTVLFVSQKTAALEVVQRRLQEIGLGDYCLEVHSTKAQKSAVLGQLRRAWHERSTPSQGTWDAATSELASLREELNGLVNALHRRRENGLSAYEAFGRVIASGGEAPLVLTWPDHLAHNETTLANLRAACRELRPVLASVGSLVDHPLQGVEATQWSPVWRDDMGAAIRAVEQTLGALRVSGQAFAEAIGLPSLLATYAGTRGLVVLGNYLVRSEARCGAAFLADGAGDLRRAVAARERFQTTKVQLLGRLTGRYRPGILDQNLGALLAEWVAAQGANFLVKGGKLKKVSAQVQPYAEGPLPPDLGPDLTGLIEVARHVKAGCLEELILARLGLPWSNPDCPASEFASAITWAEKVEQLLDILGPLSLGIDGLRDHLVHLVERQGRALADGGRIAQTYAAFAQDRARANEAMKALGVLAGRPDPEEPLAAEADWIERSCTIARRLSSGLSRAQGWCAWQAAAQSALKTGLAPLIDALEDGRIAPDRAEIAFEIAYARWWIDRVVSDDPVLRRFLPARHEDAIQRFRAADARVTELSKQVVRSRLGGGIPGATAFGADPEWGTLSHELTKKTAHMPLRKLFGKMPTALTKLTPCVMMSPLSIAQYLPPDKEPFDVVIFDEASQISPWDAIGALARAKQVVIVGDPEQLPPTNVGDRGVDDIEDGSDVTDQESILDECLAANIPRRNLDWHYRSRHESLIAFSNSRYYGGRLVTFPSPVTDDRAVRLTLVPDGVYKRGSGRVNRPEARAVVADIVRRLRDPSFSEERRSLGVVTFNGEQQRLIENLLDEQRRSYPELEPFFDRDRWHEPVFVKNLENVQGDERDAIIFSVAVGPDQTGRPVSTVSSLNKDGGHRRLNVAITRARRELVVFASMRPEQIDLGRTRARGVRDFKHFLEFAERGARALAEAFAPTGGDVESPFEAAVMAGLEARGWTVHTQIGVSGFRIDLGIVHPDAPGRYLAGVECDGATYHSSATARDRDRLREHVLTDLGWRIRRVWSTEWWMDAEGALTKLDQRLIEDLEADRAKAAAAAAEAPRDVAVEPEAVEQEHDEPTGEPEVTPPVDTGPSEPANDLEPVTDLIPQRLYADQALPVTPPAPKPEVYDDVRAYRIVDLNDLGRSVEPGRFYDASYQQALSAMVDHVLAVEGPIYEELLIKRIARAHDIQRVGPLVREAIADRIDASVARTEDDGRPVLWPRGEEPRASYPHRPASAAIRSHTDTPMPELVGIAMTLPSNASEAERARMIGQRLGLSRIEASARARFERASELARQAAVA

SEQ ID NO: 28 - protein, 210 aa, Methylobacterium sp.
MSVVLYARVSTAEQTLEHQQTQAEAAGFVFDAVVADHGESGRKPLRDRPEGRRLYDMLRTGDVLVVRWINRLGRSYEDVTGVMRELMQRGVIVRTIISNMTFDGATKDPMQRAIRDALIAFMAAAGEAELEATREAQKAGIEHARKQADQTAYRGRKPSYTRDQLTVISGMLGRGAGVSAIAAETGLSRQTIYRVQADPVEAEAALARWA

SEQ ID NO: 29 - protein, 69 aa, Methylobacterium sp.
MLSLDDIAAAAAGEERDALWRSLVEDMEEAAGRRRGGRGLVQADRPADLARALGRDRRVQPSRLARSAS

SEQ ID NO: 30 - protein, 54 aa, Methylobacterium sp.
MPVGIGIGRGDPLRPAVTRTARFSGPEGFHPGALWLAAASPLLATLLLLVRLAA

SEQ ID NO: 31 - DNA, 300 bp, Methylobacterium sp.
  source 1..300: mol_type = genomic DNA
aaggcccctcaatataaggagagattccctaccggagtgacgtttcactgcgcaagttatacagattgcaaaaatcacattggtagcacctactccccaagctacgccaatctctccaactaacggccctaccagaacagcagctactcggccaacgcttagtactgcagtagtaaggaatatcgctctgccatttccatttgcgagcaatacggcaccacataagctgaaaatagcgttaactgcataaaacggtattaatacttgcactaacgccgttgctgagacccatttttcccc

SEQ ID NO: 32 - DNA, 301 bp, Methylobacterium sp.
  source 1..301: mol_type = genomic DNA
cgcgccaagcgatcctccggcgagacatccagcgcgcgatcctccgacacgtcgaagggatgcccgttgatcgaggatcgggccttgtggcggatggctgggtaatccgccttgatcttcgctaggtcctccgcgctcatagcgccattgcgcgccggtacactgtaattggccgtccgctggaacaccgtgagatgggcggccttcgccgcgatcaccggagcggcttggatcccggtggaaccagtcccgatcaggccaacccgccggcccgtgaaatcgacatcctcatggggccatc

SEQ ID NO: 33 - DNA, 300 bp, Methylobacterium sp.
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 34 - DNA, 300 bp, Methylobacterium sp.
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 35 - DNA, 300 bp, Methylobacterium sp.
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 36 - DNA, 300 bp, Methylobacterium sp.
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 37 - DNA, 300 bp, Methylobacterium sp.
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 38 - DNA, 300 bp, Methylobacterium sp.
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 39 - DNA, 300 bp, Methylobacterium sp.
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 40 - DNA, 300 bp, Methylobacterium sp.
  source 1..300: mol_type = genomic DNA
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 41 - DNA, 300 bp, Methylobacterium sp.
  source 1..300: mol_type = genomic DNA
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 42 - DNA, 300 bp, Methylobacterium sp.
  source 1..300: mol_type = genomic DNA
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 43 - DNA, 300 bp, Methylobacterium sp.
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 44 - DNA, 300 bp, Methylobacterium sp.
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 45 - DNA, 300 bp, Methylobacterium sp.
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 46 - DNA, 300 bp, Methylobacterium sp.
  source 1..300: mol_type = genomic DNA
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 47 - DNA, 300 bp, Methylobacterium sp.
  source 1..300: mol_type = genomic DNA
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 48 - DNA, 299 bp, Methylobacterium sp.
  source 1..299: mol_type = genomic DNA
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 49 - DNA, 300 bp, Methylobacterium sp.
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 50 - DNA, 300 bp, Methylobacterium sp.
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 51 - DNA, 300 bp, Methylobacterium sp.
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 52 - DNA, 300 bp, Methylobacterium sp.
  source 1..300: mol_type = genomic DNA
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 53 - DNA, 300 bp, Methylobacterium sp.
  source 1..300: mol_type = genomic DNA
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 54 - DNA, 300 bp, Methylobacterium sp.
  source 1..300: mol_type = genomic DNA
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 55 - DNA, 300 bp, Methylobacterium sp.
  source 1..300: mol_type = genomic DNA
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 56 - DNA, 300 bp, Methylobacterium sp.
  source 1..300: mol_type = genomic DNA
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 57 - DNA, 300 bp, Methylobacterium sp.
  source 1..300: mol_type = genomic DNA
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 58 - DNA, 300 bp, Methylobacterium sp.
  source 1..300: mol_type = genomic DNA
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 59 - DNA, 300 bp, Methylobacterium sp.
  source 1..300: mol_type = genomic DNA
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 60 - DNA, 300 bp, Methylobacterium sp.
  source 1..300: mol_type = genomic DNA
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 61 - DNA, 300 bp, Methylobacterium sp.
  source 1..300: mol_type = genomic DNA
ctcaacgttgccgccttaaccgaagactaactaacataaatttcagttagccgcgaaacagaatatgatacttggtcttatacgacgcatagaggcgcattccgtcaagtcgcgcattgcagagtccccccgaaaattcgttttctgggagcgagcagaaacgtgtgcacgtgttctgtcactctattttctggtttagagcaatcatctcgcggctcagatcccgtcgttccatagagtggtttccggttcagtatgaattgtgagcagcccctgcggctatatctacttcgacgtata

SEQ ID NO: 62 - DNA, 300 bp, Methylobacterium sp.
  source 1..300: mol_type = genomic DNA
accaacaggcagaagaggcttacaaaaatgctctagatatctataatcgcgccggtctggataataggcgggaaaagtcaaatattcttatcggacttggcgatgcagctagcgcactgagcaaatttatagacgcgaaaaatttctacagtgaaggtttagcagtccgagcgcggtcatgacggtgcttgcggactagtgtctgctcgcaattcagagcagccgcttcggctggttcagtttgggtatgccctttctgtaccagcctgtgactggtctggtatgtagcccttgaccaat

SEQ ID NO: 63 - DNA, 300 bp, Methylobacterium sp.
  source 1..300: mol_type = genomic DNA
atgaagctccgtcgtgtttatgtaaagaacgttcgcagtttctacgatgctgaagaactcattctggacggagatatttctattattataggcccaaatggtggaggaaaaacaaatctacttgatgcgacgatacacttgttgagaagacatttattgcaatcgtgggctgtcgttcgccatacatacacgccaataaactattccgatcacttccaaccaaatgatcaaatcacaaactatgcgctggagaagtacagcggtcgcgaaagcgaagaccaagtcgttgagttcgatatc

SEQ ID NO: 64 - DNA, 300 bp, Methylobacterium sp.
  source 1..300: mol_type = genomic DNA
cgggggcggtctcctcatatatggagggaattaatctgaagggtcggagcccccgctacggcacacacacggcggcgggcggtgggctccttcctcatatacgcttcctgataatttcgaccgtaatcgcccgccggccgagcggccagaccttattagtccagcatcacacggtgcatcgtatcgtggagggcgaacaaccgcctaaggttcgccacgtagccatccacatccgcccttgccgctcgcgcaagggaagcagaatgcttgatagcgacttcgcgggcgtctttggtagcc

SEQ ID NO: 65 - DNA, 300 bp, Methylobacterium sp.
  source 1..300: mol_type = genomic DNA
tgtgggcgctcatggaaacgggggacgtgcttttttgatctctagccattgtcatcatccatcggtccgggtgacaggcctttacgcacataacaacggttatttcgatcagccccctcctgatcgcacaagtcagctatacagatgcttctgaaagccaacaaaggcgttacgtatctgcgtgaccccaccgagctgcgctgcggcatcgctgacagctctgtattttaattcgaggaggtggggcaatttagcctggtcaagctcgccaacgtcttccttcacatattgagacaggac

SEQ ID NO: 66 - DNA, 300 bp, Methylobacterium sp.
  source 1..300: mol_type = genomic DNA
agagtgtatttccccaagactttatctctttgctcatcacaaaacagcccgctcaagatgatatgcgctgggatttgtgccgcgacccgttgtaattgtatttacctctcggctcatgttccctgtcgccggagcgattggaggtaaagtaatctcaactttgcggcccatggctgatgctagctcagctacgcgacctaaggtaatatctttgtgtccgcggagttcgcgatgaatgaccgagcgattgacgccgatggtgcgggcaatatcggcttgagttagaccgttcttggcttc

SEQ ID NO: 67 - DNA, 300 bp, Methylobacterium sp.
  source 1..300: mol_type = genomic DNA
acctgtctcgacatcgggggcagccacgaggcgtcggccgtggtagatctcggcgaagcggtagtagtgggcgaggtccccatcgggatcgatgggggagtccggcgtaccttctccctcttctgcgattagcgacagggctcgcatggcgctgggtgcgtcagtgatggcgaacaacctactatcggggaaccaccgcctgtcgacgagctggcggtccgggtttcctttgaagacagcgtcacccagcgcttcgatcttctcaatcaaggctgcgtagaactgcccgatggtcgcgaa

SEQ ID NO: 68 - DNA, 300 bp, Methylobacterium sp.
  source 1..300: mol_type = genomic DNA
gacgtggtgacggtggcaccagtcgcccagcgaagcggctactgtactgagcccgcagtcatgtcggctgaagaaatggtcgatatgccccagggaggtcgggcgtaggtcgtacctcaaattcaccggcaggatcttgtccagcaaggcgtccgaattcgcatcgacctgacttaggaaattaccgaggtaatcgctcggtccgttcgcgtcggcggcctgcatcgccagcagcgccttatgctcggtcgtcaactcacgagcgaatattgtccaggacaggttgtcgacctcggcgac

SEQ ID NO: 69 - DNA, 300 bp, Methylobacterium sp.
  source 1..300: mol_type = genomic DNA
gagcatggcaggccaccacgcatcgcttcttcatcaccttgctcggacgacttgctgctttgtcggtctctctctcaaggacgagacgctgcgacacttgcttcgtcagagtgccaggattaatcccggtcaataccactactacgtcagctggcgtgcgccgggcggcacgagggacgtcgacgcggaacgattgattcgagacgctaactttgacgtgtacaatttagtcacgcttttccttagcgacgccgagatagcctcgttgggccgcttgctcaccgccgatgcagccgagtt

SEQ ID NO: 70 - DNA, 300 bp, Methylobacterium sp.
  source 1..300: mol_type = genomic DNA
aacgcgatgggagcagcacgtaatgggcgtcatacaagatataaaagaaggacttgaaggtagaatagataaccttaaggaaaatattgataatatcagttccagtatcacatgcacaatacaaggagtgccgaaggtcgagtcagaaagaattttgggatgtgcaaacgcttcgtttgaccaaatacagaaaatgtggatgaatggtcctacggatagggctgcatttctcgacgccgtgaaaagtttgatagaccgcaacgcaagtaatggaaaattcggagttccaggatcatattg

SEQ ID NO: 71 - DNA, 300 bp, Methylobacterium sp.
  source 1..300: mol_type = genomic DNA
ttttcctttcgcgcctatgacctgagtgcggccgaagctgtccgcttattagagttttacaaccagatgcgagcggcttagctcttcctccgttattaagcagggcgcctcttcttaggggcgccccttcatatttaatcttgtctgatgtctggcgccatatcagacaaacatcagcgttgaccttgattttgcatctacattagggttgctcccagaatggagccgccgatgtccgtcccgtacctcaagcgagagatgtggggtgtgtattacatccattggcgcgagggcggccgc

SEQ ID NO: 72 - DNA, 300 bp, Methylobacterium sp.
  source 1..300: mol_type = genomic DNA
gttggagatggtgccgtttgcaaccgtcagatcgcctactgtgaatccggtgacctcccgagagaacgtgaaggtaactgtcgtagtctcgccaactgccaaagaggtgtccgcaacggcgatggtcgctgtcggggcgcttgtttggacatcaacggtgagacccgccgaggcaggccccgtgttaccagcggtatcgacggccctagcggtgaccgtgtgcgatccagccgttagcgtcgaactggtgatgctataggtcccgccgatcgccactgccgagcccagaatggtcgtgcc

SEQ ID NO: 73 - DNA, 300 bp, Methylobacterium sp.
  source 1..300: mol_type = genomic DNA
accgaaggcgtccccggacacgaaggcctgaaacaccatatctgtggcgatcaggccgacgtggtcgcggacttcaactggcagagaatgccaggccgcttcgatttcagatgatactggtacggacataggagcggcttagctttctcagtgcaaatgtgattgattccggctcaaaaatgatcttgatcggacgagacgttttcaatccatgtcgtgttgccatcgccgatcggtgcgtcaagagacagatggcgccgaccgtagatacgcgttcgggttgcccgcaccgcttctcca

SEQ ID NO: 74 - DNA, 300 bp, Methylobacterium sp.
  source 1..300: mol_type = genomic DNA
ggaggtgtgatctgatgatgtgctggatgaaattggcggtcgagcacttgttcagcttggccagctcgacgagatcggcgtgatgctcggcgtcgatcaggatgttcagcgagaccggacgtacgcaggacttggtattagcgccgttgcgcatcagcttgcagccttgctctgcttctcagcgtgccgcgtcaggatgaccctgatgtagctgttgaggttgatgccgtaatagcctgcggactctgtgagatcccggcgaagatcgtcggcgagggtcaggcggatggtgctggtcgg

SEQ ID NO: 75 - DNA, 300 bp, Methylobacterium sp.
  source 1..300: mol_type = genomic DNA
aagtaaccgctcaacatgatcttcagcatgttgtccaacagcaggagaatacatgtaattcaccatgaccggcaagctgcgactggccattgcttccaccgcttgaatgtagcgatcgaatttcgcaaaatcagggtggaatgaaaatatcgaaccaaactgcgagccttgaatccgttctgcaaaattatcgaaaaattttcttggccgactgccgttcgaaaacattcttacgtttacatgcggcccgcctgaaacaagacagtctaccagctctgggaaatgggggtgaagggtcgg